(19) United States
(12) Patent Application Publication
(10) Pub. No.: US 2009/0196854 A1
(43) Pub. Date: Aug. 6, 2009

(54) METHODS AND COMPOSITIONS FOR USE OF CRL 5803 CELLS FOR EXPRESSION OF BIOTHERAPEUTICS AND ENCAPSULATED CELL-BASED DELIVERY

(75) Inventors: Samuel A. Clark, Barrington, RI (US); Christian Quaade, Madrid (ES); Heba Costandy, Plano, TX (US)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: KYTOS BIOSYSTEMS S.A., Madrid (ES)

(21) Appl. No.: 12/025,392

(22) Filed: Feb. 4, 2008

(51) Int. Cl. A61K 48/00; C12N 5/00; A61P 25/00

(52) U.S. Cl. 424/93.2; 435/325

(57) ABSTRACT

The use of CRL 5803 cells as a robust cell substrate for transgene expression of polypeptides is contemplated. Furthermore, use of CRL 5803 cells in cell-based delivery and methods of making and using the same in encapsulated devices are contemplated.

### FIELD OF THE INVENTION

This invention relates to methods and compositions for use in cellular therapeutic indications and to devices that comprise cells that produce therapeutically relevant factors.

### BACKGROUND

In recent years, advances in molecular biology led to the sequencing and cloning of human genes. With these advances, it became feasible to look for methods of easily producing proteins to replace or supplement those that are defective or deficient in disease states. Initially, such protein production was carried out by way of production in recombinant bacteria engineered to express such proteins, which could then be harvested and supplied to the patient in need thereof. The logical progression of production of proteins in bacteria was to introduce the genes straight into human cells. The initial gene therapy work focused on diseases caused by single-gene defects, such as cystic fibrosis, hemophilia, muscular dystrophy and sickle cell anemia. However, gene therapy provided to be more difficult than modifying simple bacteria, primarily because of the problems involved in carrying large sections of DNA and delivering it to the right site on the genome.

Nevertheless, today, significant advances in technology for vector construction and delivery have allowed gene therapy to be become a viable treatment modality. Gene therapy involves the delivery of a gene of interest to inside the cells of a subject in need of the therapy. There are two major groups of gene delivery systems used in gene therapy: viral and nonviral delivery systems. Viral delivery systems, e.g., using adenoviruses or herpes simplex II viruses, are quite efficient, but the systems suffer disadvantages of toxicity, immunogenicity of the viral components, potential risk of reversion of the virus to a replication-competent state, potential introduction of tumorigenic mutations, lack of targeting mechanism, limitations in DNA capacity and difficulty in large-scale production. Non-viral delivery systems are cationic liposome-DNA complexes, i.e., lipoplexes, liposome containing a DNA encapsulated therein along with a DNA condensing agent, or polymer complexes, i.e., polyplexes (see Shangguan et al, Gene Therapy 7:769-783, 2000). These non-viral delivery systems protect the DNA from extracellular DNases by condensation (in lipoplexes and polyplexes) or physical separation of the DNA from the extracellular environment via a lipid bilayer (in true liposomes carrying the DNA). The true liposomes of the prior art carrying the DNA require the inclusion of a DNA condensing agent, e.g., polycations of charge 3+ or higher, such as polyamines.

In another alternative, rather than delivering a gene to the inside of a cell, a cell is modified to express the gene of interest and the cell itself is retained, or “encapsulated” inside a semipermeable hydrogel or polymer membrane. In such microencapsulation techniques, a porous membrane is formed around the cells permitting the exchange of nutrients, gases, and metabolic products with the surrounding environment in which the encapsulated cells are placed. Several methods have been developed that are gentle, rapid and non-toxic and where the resulting membrane is sufficiently porous and strong to sustain the growing cell mass throughout the term of the culture.

In theory, where the encapsulated cell is one that produces a therapeutic agent, the cell can be implanted directly into the target site for site-specific, continuous, long-term, low-level delivery of the expression product of the gene of interest. Encapsulating cells in semi-permeable membranes also reduces the risk of tumor development. Furthermore, polymer-encapsulated cell transplants have lower incidences of infection, because the transplants require only a single penetration into the target site for continuous growth factor delivery. The encapsulated cells are then introduced to the appropriate site in the mammal and produce the expression product of the gene of interest at the site at which the encapsulated cell is implanted.

In practice, it has previously been demonstrated that polymer-encapsulated cells can deliver ciliary neurotrophic factor (CNTF) continuously with therapeutic efficacy in rodent models (Emerich et al., J. Neurosci., 16(16): 5168-81, 1996) and that chronic CNTF delivery into the human central nervous system (CNS) with polymer-encapsulated cells (Aebischer et al., Hum. Gene Ther., 7(7):851-60, 1996), Aebischer et al., Nat. Med., 2(6); 696-9, 1996) appears to be safe. Nevertheless, the existing technologies have a major problem in that there do not appear to be cells that have robust, long-term viability in vivo.

Indeed, the literature does not convey a consistent view as to the appropriate cell type to use for delivery of secreted proteins in the CNS. While there are published reports indicating that many cell types can be used successfully for encapsulation into microdevices for cell-based delivery of therapeutic factor, in practice, these encapsulated cells do not grow or maintain efficient function in the rigors of the in vivo environment.

As such, there remains a need for a safe, effective cell-based therapy that allows in vivo delivery of a given therapeutic agent but is able to survive the in vivo environment into which it has been implanted.

### SUMMARY OF THE INVENTION

The present invention exploits the robust properties of the CRL 5803 cell line in heterologous or exogenous gene expression for use in implantable devices. While this cell line was originally isolated as a tumorigenic cell line and is used as a cancer cell model, it has been found herein to be a surprisingly effective and safe as a neuroendocrine cell line that can be used to provide in vitro production, and in vivo delivery of therapeutic agents of interest.

The CRL5803 cells have a number of properties that facilitate the use of these cells for heterologous or exogenous gene expression of introduced DNA. CRL5803 cells grow readily at clonal densities, and are effectively sensitive to common selection drugs; these properties both increase the efficiency for selection of cells that have incorporated exogenous DNA. The CRL5803 cells are robustly transfectable, in that CRL5803 cells, in contrast to other tested cells, do not require relatively narrow parameter protocols (cell-, DNA-, or transfection carrier-concentrations or ratios), for effective transgene delivery and resultant gene expression. As used herein, the term “transgene” means a heterologous or exogenous polynucleotide that is introduced into a cell, wherein said cell does not include the polynucleotide in the absence of its introduction. The term “transgene” does not necessarily imply that the polynucleotide encodes a protein.

In some embodiments, a CRL 5803 cell is provided that has been genetically modified by transformation with one or more expression constructs comprising a nucleic acid encoding a transgene operatively linked to a promoter. The CRL 5803 cell expresses at least a first transgene, a second transgene, a third transgene and a fourth transgene, wherein at least one of said first, second, third or fourth transgenes encodes a therapeutic polypeptide and wherein said cell expresses said therapeutic polypeptide for at least 40 population doublings. In some embodiments, the cell further comprises at least one selectable marker. An exemplary therapeutic polypeptide include those therapeutic polypeptides is selected from the group consisting of a growth factor, an enzyme, a cytokine, a tumor suppressor, an apoptosis inducer, a hormone, a hematopoietic factor, a hemostasis factor, a pressor molecule, a receptor, a transporter protein, and a channel protein. In one embodiment, the first transgene is a neurotrophic factor gene, the second transgene is secreted alkaline phosphatase (SEAP) gene, the third transgene is a wild type cytosine deaminase gene, and the fourth transgene is a neurotrophic factor gene. In some embodiments, the marker gene is a neomycin phosphotransferase gene. In some embodiments, the cell is further transformed with a hygromycin phosphotransferase gene.

A multiplicity of promoters can be used effectively to confer selection gene-mediated resistance to selection drugs. In some embodiments, the promoter is heterologous to the nucleic acid encoding the therapeutic polypeptide. In other embodiments, the promoter is homologous to the nucleic acid encoding the therapeutic polypeptide. In some embodiments, the promoter is selected from the group consisting of a constitutive promoter, a tissue-specific promoter, an inducible promoter, and a non-inducible promoter. Exemplary promoters include a human cytomegalovirus (CMV) immediate early gene promoter, a SV40 early promoter, a Rous sarcoma virus long terminal repeat, a rat insulin promoter, an EF-1 α promoter, an Ubiquitin promoter, and a glyceraldehyde-3-phosphate dehydrogenase promoter.

The CRL5803 cells, in contrast to other human cells tested, are capable of relatively high level of expression for a broad range of growth factors and secreted proteins described herein (including, but not limited to, hGH, FGF-7, BDNF, VIP, and SEAP) and intracellular proteins (including but not limited to cytosine deaminase, neomycin phosphotransferase, hygromycin phosphotransferase, blasticidin-S-deaminase and bleomycin binding protein,). These findings of robust heterologous or exogenous gene expression for a number of genes suggest that receptors and other cell surface proteins would also readily be over-expressed by CRL 5803 cells. Further robustness was found in terms of promoters. The high level of transgene expression can be accomplished with a broad range of promoters, including promoters that were found to be much less effective in other cell types. The ubiquitin promoter, among those tested, provides expression levels in CRL 5803 cells that exceed those provided by the CMV promoter.

Also provided is a CRL 5803 cell that has been deposited with DSMZ under accession number DSM ACC2730.

Also provided herein are methods of treatment comprising the step of implanting in a subject a CRL5803 cell described herein, wherein the cell expresses a therapeutic polypeptide in amount effective to treat a condition treatable with the therapeutic polypeptide. In some embodiments, the cell is implanted in said subject's central nervous system, such as the subject's brain or spinal cord, for treatment of a central nervous system disorder. In some embodiments, the cell is implanted in a tumor of the subject. The methods described herein optionally further comprise administering an immunosuppressant, and/or anti-viral agent, and/or anti-bacterial agent to the subject. In some embodiments, the cell is implanted in the subject in a cell density of about 1×10⁷ cells/ml to about 1×10⁹ cells/ml.

Methods of delivering a therapeutic polypeptide to a site in a human body are also provided. In one aspect the methods comprise implanting at the site a CRL5803 cell described herein, wherein the cell expresses the therapeutic polypeptide. In some embodiments, the site is a central nervous system site, such as a brain or spinal cord site. In some embodiments, the site is a tumor.

Also provided is a method for sustaining in vitro production of a therapeutic polypeptide comprising culturing a CRL5803 cell described herein under conditions that allow production of said polypeptide for at least 40 population doublings.

In another embodiment, the invention provides a human cell engineered with a cytosine deaminase expression construct derived from a wild-type cytosine deaminase bacterial gene sequence operably linked to a promoter element, wherein the cytosine deaminase expression construct expresses a protein comprising the amino acid sequence set forth in SEQ ID NO: 11. In some embodiments, the cell is a CRL5803 cell.

Compositions comprising a cell described herein and a pharmaceutically acceptable carrier, excipient or diluent are also contemplated.

Also provided is an implantable system comprising a CRL 5803 described herein, wherein implantable system is immobilized at an implantation site to maintain said cell at the implantation site and permit diffusion of an expressed and secreted therapeutic polypeptide from said implantation site. In some embodiments, the implantation site is a central nervous system site, such as a brain or spinal cord site. In some embodiments, the implantation site is a tumor. In some embodiments, the system comprises a cell density between about 1×10⁷ cells/ml to about 1×10⁹ cells/ml. In some embodiments, the cell survives under culture conditions or in vivo in the system for at least a month with a functionality that represents at least 80% of the function expressed at the time the cells are/were introduced into the system. In some embodiments, the cell in the system expands in said system to increase in cell density and/or cell function upon implantation of the system in vivo.

Various uses of a cell described herein are provided. In one aspect, the use of a cell described herein for the in vitro production of a therapeutic polypeptide is provided. Also provided is the use of a therapeutic polypeptide expressed by a cell described herein in the manufacture of a medicament for the treatment of a condition treatable with the therapeutic polypeptide.

Another aspect includes the use of a therapeutic polypeptide expressed by a cell described herein in the manufacture of a medicament for the treatment of a CNS disorder. In some embodiments, the therapeutic polypeptide is selected from the group consisting of a growth factor, an enzyme, a cytokine, a tumor suppressor, an apoptosis inducer, a hormone, a hematopoietic factor, a hemostasis factor, a receptor, a transported protein and a channel protein.

Other features and advantages of the present invention will become apparent from the following detailed description. It should be understood, however, that the detailed description and the specific examples, while indicating preferred embodiments of the invention, are given by way of illustration only, because various changes and modifications within the spirit and scope of the invention will become apparent to those skilled in the art from this detailed description.

In addition to the foregoing, the invention includes, as an additional aspect, all embodiments of the invention narrower in scope in any way than the variations specifically mentioned above. With respect to aspects of the invention described as a genus, all individual species are individually considered separate aspects of the invention. Although the applicant(s) invented the full scope of the claims appended hereto, the claims appended hereto are not intended to encompass within their scope the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention. Additional features and variations of the invention will be apparent to those skilled in the art from the entirety of this application, and all such features are intended as aspects of the invention.

### BRIEF DESCRIPTION OF THE DRAWINGS

The following drawings form part of the present specification and are included to further illustrate aspects of the present invention. The invention may be better understood by reference to the drawings in combination with the detailed description of the specific embodiments presented herein.

FIG. 1 illustrates that some human cells, exemplified by CRL5816 cells, exhibit a glucose-dependence for growth and survival in vitro.

FIG. 2 shows growth inhibition of ARPE-19 cells plated and cultured for two weeks in 5-fluorouracil, and potential for regrowth on cessation of fluorouracil. This illustrates a pre-screen for susceptibility to the cell suicide gene cytosine deaminase.

FIG. 3 shows the detection of methanol precipitated brain-derived neurotrophic factor (BDNF) and affinity-captured transgene BDNF for CRL1440-, CRL 5803-, and ARPE-19-derived cells.

FIG. 4 compares SEAP release with the growth rate of CRL 5803 cells during the log phase of growth.

FIG. 5 shows immunoblot analysis for cytosine deaminase (CD) in cellular extracts.

FIGS. 6A and 6B show Western analyses for expression of cytosine deaminase in KB302, KB570 and KB571 cells. These cells represent modified expression vectors.

FIG. 7 shows the small size difference between amino-extended CD (SEQ ID NO: 11) from 571/10 cells and mutated, wild-type size CD (SEQ ID NO: 13) from 234/14 cells.

FIG. 8 shows Western analyses of brain-derived neurotrophic factor (BDNF) from polyclones of CRL-5803 cells transfected with multiple transgenes.

FIG. 9A shows the growth of cells housed in a device and implanted in immunosuppressed vs control rabbits for 2 weeks. FIG. 9B shows the growth of cells when removed from the CNS of immunocompromised vs control rabbits and placed in cell culture for two months.

FIG. 10A shows the growth of cells housed in a device and implanted in immunosuppressed vs control rabbits for 4 weeks. FIG. 10B shows the growth of cells when removed from the immunocompromised vs control rabbits after 4 weeks in the CNS and placed in cell culture for two months.

FIG. 11 shows metabolism of cells in devices, either cultured continuously, or implanted. The “implant” device was cultured except for the 4 weeks in the primate CNS.

FIG. 12 shows plasmid constructs B1-1 and B1-3.

FIG. 13 shows plasmid constructs C1-1 and D1-2.

### DETAILED DESCRIPTION OF EXEMPLARY EMBODIMENTS OF THE PRESENT INVENTION

The present invention is related to the discovery that CRL 5803 cells are useful for heterologous gene expression, and in the context of an encapsulation device, as a delivery cell line for encapsulated cell-based delivery of therapeutic agents. In one aspect, the therapeutic agent is selected from the group consisting of a nucleic acid and a polypeptide. While in practice the delivery of therapeutic agents using encapsulated cells has been suggested for a number of years, it has been discovered that once encapsulated many cells will not survive the rigors of the in vivo environment. In order for a cell line to be appropriate for use in cell-based delivery of a therapeutic agent, the cell line must be able to withstand the stringent in vivo conditions. For example, the cells must retain their growth characteristics in the avascular or vascular tissue cavities into which the encapsulated cells are implanted. The cells also need to be readily amenable to recombinant modification in order that production of the therapeutic agent to be introduced by the cells can be engineered into the cells (i.e., the cells can be genetically modified to express the therapeutic agent of interest). It is important that the cell line chosen has a long life span. It is desirable that once implanted the cells should remain viable and exhibit at least an 80% viability (as compared to the initial cell density of the device) for a period of at least one month in vivo in order to provide sustained delivery of the therapeutic product.

Survival in the in vivo environment is necessary for a cell that is to be used in the context of an encapsulated-cell protein delivery system. However, survival in vivo represents merely a minimal requirement, survival with little or no protein delivery will not provide an effective delivery system. The production and diffusion of the therapeutic product from the cells should be such that it is of a quantity that is sufficient to ameliorate, treat or detectably affect the disorder for which the product is being delivered. An exception may be the eye where cells that exhibit a >90% loss in protein output/implanted cell still provide some therapeutic benefit (U.S. Pat. No. 6,361,364). However, for direct delivery in other areas of the body, e.g., the CNS, a number of diseases may be expected to require microgram amounts of therapeutic peptide. However, with the cells available to date, e.g., ARPE-19 cells, there is a large decrease in secretion due to encapsulation and then secondarily to the in vivo environment (U.S. Pat. 6,361,364). As such, these cells may not provide the optimal cell for some neurodegenerative diseases.

A characteristic of CRL5803 cells that is significant in the context of implantation safety is the heterologous or exogenous expression of cytosine deaminase (CD) from a wild-type E. coli cytosine deaminase (wtCD) gene in a mammalian cell. The wtCD transgene conferred sensitivity to 5-fluorocytosine (5FC). The wtCD transgene provides a means for selective killing of CRL5803 cells by 5FC in vivo.

The CRL5803 cells were found to express a number of proteins characteristic of neuroendocrine cells (such as endoproteases-PC1 or PC2, carboxypeptidase, PAM- an amidating complex, and granule proteins-synaptophysin or chromagranin). Many of these proteins confer post-translational processing capabilities to cells, and CRL5803 cells exhibit the capacity of post-translational processing (as exemplified with BDNF) necessary to process translated proteins to active and secreted forms. A report in the literature also indicates effective glycosylation with CRL5803 cells (Lane et al., Protein Expr. Purif., 36(2):157-164, 2004).

Furthermore, as discussed herein, the CRL5803 cells were found to be capable of expressing multiple transgenes (at least seven transgenes), with stable gene expression for ≧70 population doublings. This stability is not contingent on continual selection pressure, obviating a need to include selection drugs in a manufacturing process. This stability is important for production of biotherapeutics in vitro, as well as, for in vivo delivery.

A number of growth characteristics are demonstrated that offer advantages in the context of biotherapeutic production, and/or delivery in the context of an encapsulating device. The cells can be grown to high densities (approaching tissue densities) and maintained at such densities in low FBS or low protein media. Similarly, CRL5803 cells not only survive at high densities, but can grow to high density and maintain transgene expression, while implanted in the CNS parenchyma of immunocompetent lagomorphs and primates. The survival lifespan of CRL5803 cells implanted in an encapsulation device, in vivo, is >218 days, and the total lifespan in an encapsulation device is >350 days.

Some additional properties observed with CRL 5803 cells relate to protein-free cryopreservation with efficient recovery at thaw, and capacity for room temperature shipping of cells at high density (≧10⁸ cells/cm³). These properties were noted in the course of the present studies. These properties can offer significant commercial advantages in manufacture.

In the present invention, there are described methods that were used to identify a specific cell line that is able to provide protein delivery (e.g., human transgenes can be engineered into this cell line which will then synthesize and release therapeutic protein products of those transgenes). The cell line identified is CRL 5803. In the present invention it is shown that this cell line can be used to prepare high cell-density devices that process and secrete sufficient amounts of the therapeutic product. The cell line is shown herein to retain efficient function in the device and this function is maintained even upon in vivo implantation of the device. Methods and compositions for preparing and using these devices are described in detail herein below.

### Preparation of Recombinant CRL 5803 Cells

The CRL 5803 cell line is used herein as a host cell for the delivery of any therapeutic agent in vitro, as well as to an in vivo location in a subject in need thereof. More particularly, the cell line is genetically modified to express a gene of interest. The genetically modified cell line is then encapsulated to form an implantable device or an implantable system and the device or system is implanted into the subject. It is anticipated that these cells can be used in vitro and in vivo for expression of integral membrane proteins such as receptors, or membrane associated enzymes.

In order to prepare the recombinant CRL 5803 cell line, a polynucleotide that encodes the therapeutic product of interest is incorporated into a vector that is capable of being introduced into the CRL 5803 cell line to render that cell line capable of producing the polypeptide of interest. Recombinant expression of proteins in eukaryotic host cells is well known to those of skill in the art. Preparation of vectors and techniques have been described in for example “DNA Isolation and Sequencing” (Essential Techniques Series) by Bruce A. Roe, Judy S. Crabtree and Akbar S. Khan, Published by John Wiley & Sons, 1996; Molecular Cloning: A Laboratory Manual, Joseph Sambrook (Author), David W. Russell (Author), Cold Spring Harbor Lab Press, 2000.

The vectors for the introduction of the polypeptide of interest may be plasmid or viral vectors. Plasmid expression vectors include, but are not limited to, plasmids including pBR322, pUC, pcDNA3.1 or Bluescript™. Viral vectors include, but are not limited to baculoviruses, adenoviruses, poxviruses, adenoassociated viruses (AAV), and retrovirus vectors (Price et al, Proc. Natl. Acad. Sci. USA, 84:156-160, 1987) such as the MMLV based replication incompetent vector pMV-7 (Kirschmeier et al., DNA, 7:219-225, 1988), as well as human and yeast modified chromosomes (HACs and YACs).

Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products also are provided, as is an element that links expression of the drug selection markers to expression of the polypeptide.

a. Regulatory Elements.

Typically, the expression vectors may comprise one or more regulatory elements to drive and/or enhance expression of upstream or downstream polynucleotides. These regulatory sequences are selected on the basis of the cells to be used for introduction and/or expression, and are operatively linked to a polynucleotide sequence to be expressed. The term “regulatory elements” is intended to include promoters, enhancers and other expression control elements (e.g., polyadenylation signals, TATA sites and the like). Such regulatory elements are described, for example, in Goeddel; 1990, Gene Expression Technology: Methods in Enzymology 185, Academic Press, San Diego, Calif.

Promoters and Enhancers. Throughout this application, the term “expression construct” or “expression vector” is meant to include any type of genetic construct containing a nucleic acid coding for gene products in which part or all of the nucleic acid encoding sequence is capable of being transcribed. The transcript may be translated into a protein, but it need not be. In certain embodiments, expression includes both transcription of a gene and translation of mRNA into a gene product.

The nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Promoters are composed of discrete functional modules, each consisting of approximately 7-20 bp of DNA, and containing one or more recognition sites for transcriptional activator or repressor proteins.

The particular promoter employed to control the expression of a nucleic acid sequence of interest is not believed to be important, so long as it is capable of directing the expression of the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter, the Rous sarcoma virus long terminal repeat, β-actin, rat insulin promoter, the phosphoglycerol kinase promoter and glyceraldehyde-3-phosphate dehydrogenase promoter, all of which are promoters well known and readily available to those of skill in the art, can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well known properties, the level and pattern of expression of the protein of interest following transfection or transformation can be optimized. The ubiquitin promoter is particularly preferred in certain examples in the present invention.

Inducible promoter systems may be used in the present invention, e.g., inducible ecdysone system (Invitrogen, Carlsbad, Calif.), which is designed to allow regulated expression of a gene of interest in mammalian cells. Another inducible system that would be useful is the Tet-Off™ or Tet-On™ system (Clontech, Palo Alto, Calif.) originally developed by Gossen and Bujard (Gossen and Bujard, Proc Natl Acad Sci USA. 89(12):5547-51, 1992; Gossen et al., Science, 268(5218):1766-69, 1995).

In some circumstances, it may be desirable to regulate expression of a transgene in a gene therapy vector. For example, different viral promoters with varying strengths of activity may be utilized depending on the level of expression desired. In mammalian cells, the CMV immediate early promoter is often used to provide strong transcriptional activation. Modified versions of the CMV promoter that are less potent have also been used when reduced levels of expression of the transgene are desired. When expression of a transgene in hematopoietic cells is desired, retroviral promoters such as the LTRs from MLV or MMTV are often used. Other viral promoters that may be used depending on the desired effect include SV40, RSV LTR, HIV-1 and HIV-2 LTR, adenovirus promoters such as from the E1A, E2A, or MLP region, AAV LTR, cauliflower mosaic virus, HSV-TK, and avian sarcoma virus.

Similarly tissue specific promoters may be used to effect transcription in specific tissues or cells so as to reduce potential toxicity or undesirable effects to non-targeted tissues. For example, promoters such as the PSA, probasin, prostatic acid phosphatase or prostate-specific glandular kallikrein (hK2) may be used to target gene expression in the prostate.

In certain indications, it may be desirable to activate transcription at specific times after implantation of a CRL 5803 cell-containing device in vivo. This may be done with such promoters as those that are hormone or cytokine regulatable. For example in gene therapy applications where the indication is a gonadal tissue where specific steroids are produced or routed to, use of androgen or estrogen regulated promoters may be advantageous. Such promoters that are hormone regulatable include MMTV, MT-1, ecdysone and RuBisco. Other hormone regulated promoters such as those responsive to thyroid, pituitary and adrenal hormones are expected to be useful in the present invention. Cytokine and inflammatory protein responsive promoters that could be used include K and T Kininogen (Kageyama et al., J. Biol. Chem., 262(5):2345-51, 1987), c-fos, TNF-alpha, C-reactive protein (Arcone et al., Nucleic Acids Res., 16(8):3195-3207, 1988), haptoglobin (Oliviero et al., EMBO J., 6(7):1905-12, 1987), serum amyloid A2, C/EBP alpha, IL-1, IL-6 (Poli and Cortese, Proc. Natl. Acad. Sci. USA, 86(21):8202-6, 1989), Complement C3 (Wilson et al., Mol. Cell. Biol., 10(12):6181-91, 1990), IL-8, alpha-1 acid glycoprotein (Prowse and Baumann, Mol. Cell. Biol., 8(1):42-51, 1988), alpha-1 antitypsin, lipoprotein lipase (Zechner et al., Mol. Cell. Biol., 8(6):2394-401, 1988), angiotensinogen (Ron et al., Mol. Cell. Biol., 11(5):2887-95, 1991), fibrinogen, c-jun (inducible by phorbol esters, TNF-alpha, UV radiation, retinoic acid, and hydrogen peroxide), collagenase (induced by phorbol esters and retinoic acid), metallothionein (heavy metal and glucocorticoid inducible), Stromelysin (inducible by phorbol ester, interleukin-1 and EGF), alpha-2 macroglobulin and alpha-1 antichymotrypsin.

It is envisioned that cell cycle regulatable promoters may be useful in the present invention. For example, in a bicistronic gene therapy vector, use of a strong CMV promoter to drive expression of a first gene such as p16 that arrests cells in the G1 phase could be followed by expression of a second gene such as p53 under the control of a promoter that is active in the G1 phase of the cell cycle, thus providing a “second hit” that would push the cell into apoptosis. Other promoters such as those of various cyclins, PCNA, galectin-3, E2F1, p53 and BRCA1 could be used.

It is envisioned that any of the above promoters alone or in combination with another may be useful according to the present invention depending on the action desired. In addition, this list of promoters should not be construed to be exhaustive or limiting, and those of skill in the art will know of other promoters that may be used in conjunction with the promoters and methods disclosed herein.

Another regulatory element contemplated for use in the present invention is an enhancer. Enhancers are genetic elements that increase transcription from a promoter located at a distant position on the same molecule of DNA. Enhancers are organized much like promoters. That is, enhancers are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization. Enhancers useful in the present invention are well known to those of skill in the art and will depend on the particular expression system being employed (Scharf D et al Results Probl. Cell. Differ., 20:125-62, 1994; Bitter et al., Methods in Enzymol., 153:516-544, 1987).

Polyadenylation Signals. Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human or bovine growth hormone and SV40 polyadenylation signals. Also contemplated as an element of the expression cassette is a terminator. These elements can serve to enhance message levels and to minimize read through from the cassette into other sequences.

IRES. In certain embodiments of the invention, the use of internal ribosome entry site (IRES) elements is contemplated to create multigene, or polycistronic, messages. IRES elements are able to bypass the ribosome scanning model of 5′ methylated Cap dependent translation and begin translation at internal sites (Pelletier and Sonenberg, Nature, 334:320-325, 1988). IRES elements from two members of the picornavirus family (poliovirus and encephalomyocarditis) have been described (Pelletier and Sonenberg, 1988 supra), as well an IRES from a mammalian message (Macejak and Sarnow, Nature, 353:90-94, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Any heterologous open reading frame can be linked to IRES elements. This includes genes for secreted proteins, multi-subunit proteins, encoded by independent genes, intracellular or membrane-bound proteins and selectable markers. In this way, expression of several proteins can be simultaneously engineered into a cell with a single construct and a single selectable marker.

b. Delivery of Expression Vectors.

There are a number of ways in which expression constructs may be introduced into cells. In certain embodiments of the invention, the expression construct comprises a virus or engineered construct derived from a viral genome. In other embodiments, non-viral delivery is contemplated. The ability of certain viruses to enter cells via receptor-mediated endocytosis, to integrate into host cell genome and express viral genes stably and efficiently have made them attractive candidates for the transfer of foreign genes into mammalian cells (Ridgeway, In: Rodriguez R L, Denhardt D T, ed. Vectors: A survey of molecular cloning vectors and their uses. Stoneham: Butterworth, 467-492, 1988; Nicolas and Rubenstein, In: Vectors: A survey of molecular cloning vectors and their uses, Rodriguez & Denhardt (eds.), Stoneham: Butterworth, 493-513, 1988; Baichwal and Sugden, In: Gene Transfer, Kucherlapati R, ed., New York, Plenum Press, 117-148, 1986; Temin, In: gene Transfer, Kucherlapati (ed.), New York: Plenum Press, 149-188, 1986). The first viruses used as gene vectors were DNA viruses including the papovaviruses (simian virus 40, bovine papilloma virus, and polyoma) (Ridgeway, 1988 supra; Baichwal and Sugden, 1986 supra) and adenoviruses (Ridgeway, 1988 supra; Baichwal and Sugden, 1986 supra). These have a relatively low capacity for foreign DNA sequences and have a restricted host spectrum. Furthermore, their oncogenic potential and cytopathic effects in permissive cells raise safety concerns. They can accommodate only up to 8 kb of foreign genetic material but can be readily introduced in a variety of cell lines and laboratory animals (Nicolas and Rubenstein, 1988 supra; Temin, 1986 supra).

It is now widely recognized that DNA may be introduced into a cell using a variety of viral vectors. In such embodiments, expression constructs comprising viral vectors containing the genes of interest may be adenoviral (see for example, U.S. Pat. No. 5,824,544; U.S. Pat. No. 5,707,618; U.S. Pat. No. 5,693,509; U.S. Pat. No. 5,670,488; U.S. Pat. No. 5,585,362; each incorporated herein by reference), retroviral (see for example, U.S. Pat. No. 5,888,502; U.S. Pat. No. 5,830,725; U.S. Pat. No. 5,770,414; U.S. Pat. No. 5,686,278; U.S. Pat. No. 4,861,719 each incorporated herein by reference), adeno-associated viral (see for example, U.S. Pat. No. 5,474,935; U.S. Pat. No. 5,139,941; U.S. Pat. No. 5,622,856; U.S. Pat. No. 5,658,776; U.S. Pat. No. 5,773,289; U.S. Pat. No. 5,789,390; U.S. Pat. No. 5,834,441; U.S. Pat. No. 5,863,541; U.S. Pat. No. 5,851,521; U.S. Pat. No. 5,252,479 each incorporated herein by reference), an adenoviral-adenoassociated viral hybrid (see for example, U.S. Pat. No. 5,856,152 incorporated herein by reference) or a vaccinia viral or a herpesviral (see for example, U.S. Pat. No. 5,879,934; U.S. Pat. No. 5,849,571; U.S. Pat. No. 5,830,727; U.S. Pat. No. 5,661,033; U.S. Pat. No. 5,328,688 each incorporated herein by reference) vector.

There are a number of alternatives to viral transfer of genetic constructs. This section provides a discussion of methods and compositions of non-viral gene transfer. DNA constructs of the present invention are generally delivered to a cell, and in certain situations, the nucleic acid or the protein to be transferred may be transferred using non-viral methods.

Several non-viral methods for the transfer of expression constructs into cultured mammalian cells are contemplated by the present invention. These include calcium phosphate precipitation (Graham and Van Der Eb, Virology, 52:456-467, 1973; Chen and Okayama, Mol. Cell Biol., 7:2745-2752, 1987; Rippe et al., Mol. Cell Biol., 10:689-695, 1990) DEAE-dextran (Gopal, Mol. Cell Biol., 5:1188-1190, 1985), electroporation (Tur-Kaspa et al., Mol. Cell Biol., 6:716-718, 1986; Potter et al., Proc. Nat. Acad. Sci. USA, 81:7161-7165, 1984), direct microinjection (Harland and Weintraub, J. Cell Biol., 101:1094-1099, 1985), DNA-loaded liposomes (Nicolau and Sene, Biochim. Biophys. Acta, 721:185-190, 1982; Fraley et al., Proc. Natl. Acad. Sci. USA, 76:3348-3352, 1979; Felgner, Sci Am. 276(6):102-6, 1997; Felgner, Hum Gene Ther. 7(15):1791-3, 1996), cell sonication (Fechheimer et al., Proc. Natl. Acad. Sci. USA, 84:8463-8467, 1987), gene bombardment using high velocity microprojectiles (Yang et al., Proc. Natl. Acad. Sci USA, 87:9568-9572, 1990), and receptor-mediated transfection (Wu and Wu, J. Biol. Chem., 262:4429-4432, 1987; Wu and Wu, Biochemistry, 27:887-892, 1988).

Once the construct has been delivered into the CRL 5803 cell, the nucleic acid encoding the therapeutic gene (or therapeutic polypeptide) may be positioned and expressed at different sites. In certain embodiments, the nucleic acid encoding the therapeutic gene (or therapeutic polypeptide) may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In a particular embodiment of the invention, the expression construct may be entrapped in a liposome. The addition of DNA to cationic liposomes causes a topological transition from liposomes to optically birefringent liquid-crystalline condensed globules (Radler et al., Science, 275(5301):810-4, 1997). These DNA-lipid complexes are potential non-viral vectors for use in gene therapy and delivery. Liposome-mediated nucleic acid delivery and expression of foreign DNA in vitro has been very successful. Also contemplated in the present invention are various commercial approaches involving “lipofection” technology. Complexing the liposome with a hemagglutinating virus (HVJ) may facilitate fusion with the cell membrane and promote cell entry of liposome-encapsulated DNA (Kaneda et al., Science, 243:375-378, 1989). In other embodiments, the liposome may be complexed or employed in conjunction with nuclear nonhistone chromosomal proteins (HMG-1) (Kato et al., J. Biol. Chem., 266:3361-3364, 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention.

Other vector delivery systems which can be employed to deliver a nucleic acid encoding a therapeutic gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific.

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) and transferrin (Wagner et al., Proc. Natl. Acad Sci. USA, 87(9):3410-3414, 1990). Recently, a synthetic neoglycoprotein, which recognizes the same receptor as ASOR, has been used as a gene delivery vehicle (Ferkol et al., FASEB J., 7:1081-1091, 1993; Perales et al., Proc. Natl. Acad. Sci., USA 91:4086-4090, 1994) and epidermal growth factor (EGF) has also been used to deliver genes to squamous carcinoma cells (Myers, EPO 0273085).

In other embodiments, the delivery vehicle may comprise a ligand and a liposome. Thus, it is feasible that a nucleic acid encoding a therapeutic gene also may be specifically delivered into a particular cell type by any number of receptor-ligand systems with or without liposomes.

In another embodiment of the invention, the expression construct may simply consist of naked recombinant DNA or plasmids. Transfer of the construct may be performed by any of the methods mentioned above which physically or chemically permeabilize the cell membrane. This is applicable particularly for transfer in vitro, however, it may be applied for in vivo use as well. (Dubensky et al. Proc. Nat. Acad. Sci. USA, 81:7529-7533, 1984; Benvenisty and Neshif Proc. Nat. Acad. Sci. USA, 83:9551-9555, 1986).

Another embodiment of the invention for transferring a naked DNA expression construct into cells may involve particle bombardment. This method depends on the ability to accelerate DNA coated microprojectiles to a high velocity allowing them to pierce cell membranes and enter cells without killing them (Klein et al., Proc Natl Acad Sci U.S.A. 1988 June;85(12):4305-4309. Several devices for accelerating small particles have been developed. One such device relies on a high voltage discharge to generate an electrical current, which in turn provides the motive force (Yang et al., Proc. Natl. Acad. Sci USA, 87:9568-9572, 1990). The microprojectiles used have consisted of biologically inert substances such as tungsten or gold beads.

The recombinant CRL 5803 produced using the above methods can be grown in standard cell culture and tested for appropriate protein production. The cells can then be used to prepare implantable devices or implantable systems of the present invention.

### Implantable Devices or Implantable Systems Comprising CRL 5803 Cells

As noted above, the CRL 5803 cells can be used to prepare implantable devices or implantable systems in which the cells are encapsulated in microporous membranes. In one embodiment, the CRL 5803 cells in the implantable devices or implantable systems produce microgram quantities of therapeutic protein.

These devices can advantageously be implanted directly into the target site for site-specific, continuous, long-term, low-level delivery of the desired factors. In such microencapsulation techniques, a porous membrane is formed around the cells permitting the exchange of nutrients, gases, and metabolic products with the surrounding environment in which the encapsulated cells are placed. Several methods have been developed that are gentle, rapid and non-toxic and where the resulting membrane is sufficiently porous and strong to sustain the growing cell mass throughout the term of the culture. Such methods use soluble alginate gelled by droplet contact with a calcium-containing solution. Lim, U.S. Pat. No. 4,352,883, for (incorporated herein by reference,) describes cells concentrated in an approximately 1% solution of sodium alginate which are forced through a small orifice, forming droplets, and breaking free into an approximately 1% calcium chloride solution. The droplets are then cast in a layer of polyamino acid that ionically bonds to the surface alginate. Finally the alginate is reliquefied by treating the droplet in a chelating agent to remove the calcium ions. Other methods use cells in a calcium solution to be dropped into an alginate solution, thus creating a hollow alginate sphere. A similar approach involves cells in a chitosan solution dropped into alginate, also creating hollow spheres.

U.S. Pat. No. 6,911,227, the disclosure of which is incorporated herein by reference in its entirety, discloses a variety of encapsulation methods that may be used in the present invention. U.S. Pat. No. 6,337,088, the disclosure of which is incorporated herein by reference in its entirety also provides teachings of microencapsulation that may be used herein.

Other methods for encapsulating or otherwise immobilizing biologically active materials, e.g. viable cells, have been disclosed which involve suspending the biologically active material in a gel composition and incorporating the gel material into the pores of a semi-permeable or permeable structure, or reacting the gel material to form a porous polymeric coating over the gel material. For example, U.S. Pat. No. 5,116,747, the disclosure of which is incorporated herein by reference in its entirety, discloses the immobilization of cells and other biologically active materials within a semipermeable membrane or microcapsule composed of an anionic polymer such as alginate induced to gel in the presence of calcium and/or a polymeric polycation such as chitosan.

The cells can be encapsulated as described in U.S. Pat. No. 4,663,286, the disclosure of which is incorporated herein by reference in its entirety, which discloses the encapsulation of cells within a semipermeable membrane, by suspending the cells in a solution of a water-soluble polyanionic polymer, preferably alginate salts, forming droplets, and gelling the polyanion with a polyvalent polycation such as a polypeptide, a protein or a polyaminated polysaccharide, preferably polylysine, polyarginine, or polyomithine. U.S. Pat. No. 4,663,286 further teaches controlling the porosity and permeability of the disclosed compositions to molecules ranging from about 60,000 to about 900,000 Daltons by changing the degree of hydration of the polymer. Incubation in saline or chelating agents increases hydration and expands the gels, whereas incubation in calcium chloride contracts the gel mass. Increases in charge density of the polycationic membrane generally produces smaller pores. Increases in the molecular weight of the polycationic polymer generally produces a thicker, less permeable membrane.

U.S. Pat. No. 6,790,455, the disclosure of which is incorporated herein by reference in its entirety, describes a cell delivery system that comprises a biodegradable and/or bioabsorbable fibrous matrix containing at least about 20 weight percent of fibers having fiber diameters in the range of about 10 up to about 1000 nanometers, formed by electrospinning fibers of a biodegradable and/or bioabsorbable fiberizable material, and viable cells physically associated with said matrix as a carrier whereby said cells are contained and released at a controlled rate. The polymer used therein is made of a monomer selected from the group consisting of a glycolide, lactide, dioxanone, caprolactone, trimethylene carbonate, ethylene glycol and lysine.

Microencapsulation of the CRL 5803 cells generally involves three steps: (a) generating microcapsules enclosing the cells (e.g., by forming droplets of cell-containing liquid alginate followed by exposure to a solution of calcium chloride to form a solid gel), (b) coating the resulting gelled spheres with additional outer coatings (e.g., outer coatings comprising polylysine and/or polyomithine or any other monomer typically used in microencapsulation) to form a semipermeable membrane; and (c) liquefying the original core gel (e.g., by chelation using a solution of sodium citrate). The three steps are typically separated by washings in normal saline.

One typical method of microencapsulating cells is the alginate-polyamino acid technique. Briefly, cells are suspended in sodium alginate in saline, and droplets containing the cells are produced. Droplets of cell-containing alginate flow into calcium chloride in saline. The negatively charged alginate droplets bind calcium and form a calcium alginate gel. The microcapsules are washed in saline and incubated with poly-L-lysine or poly-L-ornithine (or combinations thereof); the positively charged poly-l-lysine and/or poly-L-ornithine displaces calcium ions and binds (ionic) negatively charged alginate, producing an outer poly-electrolyte membrane. A final coating of sodium alginate may be added by washing the microcapsules with a solution of sodium alginate, which ionically bonds to the poly-L-lysine and/or poly-L-ornithine layer. See U.S. Pat. No. 4,391,909 to Lim et al (all US patents referenced herein are intended to be incorporated herein in their entirety). This technique produces what has been termed a “single-wall” microcapsule. The microcapsules produced are essentially round, small, and uniform in size. Wolters et al., J. Appli Biomater. 3:281-286 (1992).

When desired, the alginate-polylysine microcapsules can then be incubated in sodium citrate to solubilize any calcium alginate that has not reacted with poly-1-lysine, i.e., to solubilize the internal core of sodium alginate containing the CRL 5803 cells, thus producing a microcapsule with a liquefied cell-containing core portion. See Lim and Sun, Science 210:908 (1980). Such microcapsules are referred to herein as having “chelated”, “hollow” or “liquid” cores.

A “double-wall” microcapsule is produced using the same procedure as for single-wall microcapsules, but before incubation with sodium citrate, the microcapsules are again incubated with poly-1-lysine and sodium alginate.

Alginates are linear polymers of mannuronic and guluronic acid residues. Monovalent cation alginate salts, e.g., Na-alginate, are generally soluble. Divalent cations such as Ca++, Ba++or Sr++ tend to interact with guluronate, providing crosslinking and forming stable alginate gels. Alginate encapsulation techniques typically take advantage of the gelling of alginate in the presence of divalent cation solutions. Alginate encapsulation of cells generally involves suspending the cells to be encapsulated in a solution of a monovalent cation alginate salt, generating droplets of this solution, and contacting the droplets with a solution of divalent cations. The divalent cations interact with the alginate at the phase transition between the droplet and the divalent cation solution, resulting in the formation of a stable alginate gel matrix being formed. A variation of this technique is reported in U.S. Pat. No. 5,738,876, wherein the cell is suffused with a solution of multivalent ions (e.g., divalent cations) and then suspended in a solution of gelling polymer (e.g., alginate), to provide a coating of the polymer.

Chelation of the alginate (degelling) core solubilizes the internal structural support of the capsule, may adversely affect the durability of the microcapsule, and is a harsh treatment of the encapsulated living cells. Degelling of the core may also cause leaching out of the unbound poly-lysine or solubilized alginate, resulting in a fibrotic reaction to the implanted microcapsule. The effect of core liquidation on glucose-stimulated insulin secretion by the encapsulated cells has been studied. Fritschy et al., Diabetes 40:37 (1991).

Alginate/polycation encapsulation procedures are simple and rapid, and represent a promising method for CRL 5803 cell encapsulation for clinical treatment of diabetes. Many variations of this basic encapsulation method have been described in patents and the scientific literature. Chang et al., U.S. Pat. No. 5,084,350 discloses microcapsules enclosed in a larger matrix, where the microcapsules are liquefied once the microcapsules are within the larger matrix. Tsang et al., U.S. Pat. No. 4,663,286 discloses encapsulation using an alginate polymer, where the gel layer is cross-linked with a polycationic polymer such as polylysine, and a second layer formed using a second polycationic polymer (such as polyomithine); the second layer can then be coated by alginate. U.S. Pat. No. 5,762,959 to Soon-Shiong et al. discloses a microcapsule having a solid (non-chelated) alginate gel core of a defined ratio of calcium/barium alginates, with polymer material in the core.

In specific embodiments, polymers used for the microencapsulation devices include, but are not limited to collagen, elastin, hyaluronic acid and derivatives, sodium alginate and derivatives, chitosan and derivatives gelatin, starch, cellulose polymers (for example methylcellulose, hydroxypropylcellulose, hydroxypropylmethylcellulose, carboxymethylcellulose, cellulose acetate phthalate, cellulose acetate succinate, hydroxypropylmethylcellulose phthalate), casein, dextran and derivatives, polysaccharides, poly(caprolactone), fibrinogen, poly(hydroxyl acids), poly(L-lactide) poly(D,L lactide), poly(D,L-lactide-co-glycolide), poly(L-lactide-co-glycolide), copolymers of lactic acid and glycolic acid, copolymers of .epsilon.-caprolactone and lactide, copolymers of glycolide and .epsilon.-caprolactone, copolymers of lactide and 1,4-dioxane-2-one, polymers and copolymers that include one or more of the residue units of the monomers D-lactide, L-lactide, D,L-lactide, glycolide, epsilon.-caprolactone, trimethylene carbonate, 1,4-dioxane-2-one or 1,5-dioxepan-2-one, poly(glycolide), poly(hydroxybutyrate), poly(alkylcarbonate) and poly(orthoesters), polyesters, poly(hydroxyvaleric acid), polydioxanone, poly(ethylene terephthalate), poly(malic acid), poly(tartronic acid), polyanhydrides, polyphosphazenes, poly(amino acids). The polymers used herein may be copolymers of the above polymers as well as blends and combinations of the above polymers. (see generally, Illum, L., Davids, S. S. (eds.) “Polymers in Controlled Drug Delivery” Wright, Bristol, 1987; Arshady, J. Controlled Release 17:1-22, 1991; Pitt, Int. J. Phar. 59:173-196, 1990; Holland et al., J. Controlled Release 4:155-0180, 1986).

The polymers may be combinations of biodegradable and non-degradable polymers. Further examples of polymers that may be used are either anionic (e.g., alginate, carrageenin, hyaluronic acid, dextran sulfate, chondroitin sulfate, carboxymethyl dextran, carboxymethyl cellulose and poly(acrylic acid)), or cationic (e.g., chitosan, poly-1-lysine, polyethylenimine, and poly(allyl amine)) (see generally, Dunn et al., J. Applied Polymer Sci. 50:353, 1993; Cascone et al., J. Materials Sci.: Materials in Medicine 5:770, 1994; Shiraishi et al., Biol. Pharm. Bull. 16:1164, 1993; Thacharodi and Rao, Int'l J. Pharm. 120:115, 1995; Miyazaki et al., Int'l J. Pharm. 118:257, 1995). Preferred polymers (including copolymers and blends of these polymers) include poly(ethylene-co-vinyl acetate), poly(carbonate urethanes), poly(hydroxyl acids) (e.g., poly(D,L-lactic acid) oligomers and polymers, poly(L-lactic acid) oligomers and polymers, poly(D-lactic acid) oligomers and polymers, poly(glycolic acid), copolymers of lactic acid and glycolic acid, copolymers of lactide and glycolide, poly(caprolactone), copolymers of lactide or glycolide and ε-caprolactone), poly(valerolactone), poly(anhydrides), copolymers prepared from caprolactone and/or lactide and/or glycolide and/or polyethylene glycol.

In specific embodiments, the polymers encapsulating the cells are molded or otherwise formed into flexible compositions that can be used as “patches.” Such “patches” are seeded with CRL 5803 cells so that they can act as cellular tissue patches or tissue grafts.

Such a patch may be formulated into any shape or configuration appropriate for maintaining biological activity and providing access for delivery of the product or function, including for example, cylindrical, rectangular, disk-shaped, patch-shaped, ovoid, stellate, or spherical. Moreover, the capsule can be coiled or wrapped into a mesh-like or nested structure. If the capsule is to be retrieved after it is implanted, configurations which tend to lead to migration of the capsules from the site of implantation, such as spherical capsules small enough to travel in the recipient host's blood vessels, are not preferred. Certain shapes, such as rectangles, patches, disks, cylinders, and flat sheets offer greater structural integrity and are preferable where retrieval is desired.

U.S. Pat. Nos. 5,801,033 and 5,573,934, the disclosures of which is incorporated herein by reference in their entireties, describe alginate/polylysine microspheres having a final polymeric coating (e.g., polyethylene glycol (PEG)); Sawhney et al., Biomaterials 13:863 (1991) describe alginatelpolylysine microcapsules incorporating a graft copolymer of poly-l-lysine and polyethylene oxide on the microcapsule surface, to improve biocompatibility; U.S. Pat. No. 5,380,536, the disclosure of which is incorporated herein by reference in its entirety, describes microcapsules with an outermost layer of water soluble non-ionic polymers such as polyethylene(oxide). U.S. Pat. No. 5,227,298, the disclosure of which is incorporated herein by reference in its entirety, describes a method for providing a second alginate gel coating to cells already coated with polylysine alginate; both alginate coatings are stabilized with polylysine. U.S. Pat. No. 5,578,314, the disclosure of which is incorporated herein by reference in its entirety, provides a method for microencapsulation using multiple coatings of purified alginate.

U.S. Pat. No. 5,693,514, the disclosure of which is incorporated herein by reference in its entirety, reports the use of a non-fibrogenic alginate, where the outer surface of the alginate coating is reacted with alkaline earth metal cations comprising calcium ions and/or magnesium ions, to form an alkaline earth metal alginate coating. The outer surface of the alginate coating is not reacted with polylysine.

U.S. Pat. No. 5,846,530, the disclosure of which is incorporated herein by reference in its entirety, describes microcapsules containing cells that have been individually coated with polymerizable alginate, or polymerizable polycations such as polylysine, prior to encapsulation.

The methods of the present invention are intended for use with any microcapsule that contains living CRL 5803 cells secreting a desirable biological substance, where the microcapsule comprises an inner gel or liquid core containing the cells of interest, or a liquid core containing the cells of interest, bounded by a semi-permeable membrane surrounding the cell-containing core. The inner core is preferably composed of a water-soluble gelling agent; preferably the water-soluble gelling agent comprises plural groups that can be ionized to form anionic or cationic groups. The presence of such groups in the gel allows the surface of the gel bead to be cross-linked to produce a membrane, when exposed to polymers containing multiple functionalities having a charge opposite to that of the gel.

Cells suspended in a gellable medium (such as alginate) may be formed into droplets using any suitable method as is known in the art, including but not limited to emulsification (see e.g., U.S. Pat. No. 4,352,883), extrusion from a needle (see, e.g., U.S. Pat. No. 4,407,957; Nigam et al., Biotechnology Techniques 2:271-276 (1988)), use of a spray nozzle (Plunkett et al., Laboratory Investigation 62:510-517 (1990)), or use of a needle and pulsed electrical electrostatic voltage (see, e.g., U.S. Pat. Nos. 4,789,550; 5,656,468).

The water-soluble gelling agent is preferably a polysaccharide gum, and more preferably a polyanionic polymer. An exemplary water-soluble gelling agent is an alkali metal alginate such as sodium alginate. The gelling agent preferably has free acid functional groups and the semi-permeable membrane is formed by contacting the gel with a polymer having free amino functional groups with cationic charge, to form permanent crosslinks between the free amino acids of the polymer and the acid functional groups. Preferred polymers include polylysine, polyethylenimine, and polyarginine. A particularly preferred microcapsule contains cells immobilized in a core of alginate with a poly-lysine coating; such microcapsules may comprise an additional external alginate layer to form a multi-layer alginate-polylysine-alginate microcapsule. See U.S. Pat. No. 4,391,909 the contents of which are incorporated by reference herein in their entirety.

When desired, the microcapsules are treated or incubated with a physiologically acceptable salt such as sodium sulfate or like agents, in order to increase the durability of the microcapsule, while retaining or not unduly damaging the physiological responsiveness of the cells contained in the microcapsules. By “physiologically acceptable salt” is meant a salt that is not unduly deleterious to the physiological responsiveness of the cells encapsulated in the microcapsules. In general, such salts are salts that have an anion that binds calcium ions sufficiently to stabilize the capsule, without substantially damaging the function and/or viability of the cells contained therein. Sulfate salts, such as sodium sulfate and potassium sulfate, are preferred, and sodium sulfate is most preferred. The incubation step is carried out in an aqueous solution containing the physiological salt in an amount effective to stabilize the capsules, without substantially damaging the function and/or viability of the cells contained therein as described above. In general, the salt is included in an amount of from about 0.1 or 1 milliMolar up to about 20 or 100 millimolar, most preferably about 2 to 10 millimolar. The duration of the incubation step is not critical, and may be from about 1 or 10 minutes to about 1 or 2 hours, or more (e.g., over night). The temperature at which the incubation step is carried out is likewise not critical, and is typically from about 4° C. up to about 37° C., with room temperature (about 21° C.) preferred.

When desired, liquefaction of the core gel are carried out by any suitable method as is known in the art, such as ion exchange or chelation of calcium ion by sodium citrate or EDTA. Microcapsules useful in the present invention thus have at least one semipermeable surface membrane surrounding a cell-containing core. The surface membrane permits the diffusion of nutrients, biologically active molecules and other selected products through the surface membrane and into the microcapsule core. The surface membrane contains pores of a size that determines the molecular weight cut-off of the membrane.

Components of the biocompatible material may include an internal cell-supporting scaffolding. Preferably, the transformed cells are seeded onto the scaffolding, which is encapsulated by the permselective membrane. The filamentous cell-supporting scaffold may be made from any biocompatible material selected from the group consisting of acrylic, polyester, polyethylene, polypropylene polyacetonitrile, polyethylene teraphthalate, nylon, polyamides, polyurethanes, polybutester, silk, cotton, chitin, carbon, or biocompatible metals. Also, bonded fiber structures can be used for cell implantation (U.S. Pat. No. 5,512,600, incorporated by reference). Biodegradable polymers include those comprised of poly(lactic acid) PLA, poly(lactic-coglycolic acid) PLGA, and poly(glycolic acid) PGA and their equivalents. Foam scaffolds have been used to provide surfaces onto which transplanted cells may adhere (PCT International patent application Ser. No. 98/05304, incorporated by reference). Woven mesh tubes have been used as vascular grafts (PCT International patent application WO 99/52573, incorporated by reference). Additionally, the core can be composed of an immobilizing matrix formed from a hydrogel, which stabilizes the position of the cells. A hydrogel is a 3-dimensional network of cross-linked hydrophilic polymers in the form of a gel, substantially composed of water.

The scaffolding can be coated with extracellular matrix (ECM) molecules. Suitable examples of extracellular matrix molecules include, for example, collagen, laminin, and fibronectin. The surface of the scaffolding can also be modified by treating it with plasma irradiation to impart charge to enhance adhesion of CRL-5803 cells.

Various polymers and polymer blends can be used to manufacture the surrounding semipermeable membrane, including polyacrylates (including acrylic copolymers), polyvinylidenes, polyvinyl chloride copolymers, polyurethanes, polystyrenes, polyamides, cellulose acetates, cellulose nitrates, polysulfones (including polyether sulfones), polyphosphazenes, polyacrylonitriles, poly(acrylonitrile/covinyl chloride), as well as derivatives, copolymers and mixtures thereof. Preferably, the surrounding semipermeable membrane is a biocompatible semipermeable hollow fiber membrane. Such membranes, and methods of making them are disclosed by U.S. Pat. Nos. 5,284,761 and 5,158,881, incorporated by reference. The surrounding semipermeable membrane is formed from a polyether sulfone hollow fiber, such as those described by U.S. Pat. No. 4,976,859 or U.S. Pat. No. 4,968,733, incorporated by reference. An alternate surrounding semipermeable membrane material is poly(acrylonitrile/covinyl chloride).

As used herein, a “poly-amino acid-alginate microsphere” refers to a capsule of less than 2 mm in diameter having an inner core of cell-containing alginate bounded by a semi-permeable membrane formed by alginate and poly-1-lysine. Viable cells encapsulated using an anionic polymer such as alginate to provide a gel layer, where the gel layer is subsequently cross-linked with a polycationic polymer (e.g., an amino acid polymer such as polylysine. See e.g., U.S. Pat. Nos. 4,806,355, 4,689,293 and 4,673,566; U.S. Pat. Nos. 4,409,331, 4,407,957, 4,391,909 and 4,352,883; U.S. Pat. Nos. 4,749,620 and 4,744,933 to Rha et al.; and U.S. Pat. No. 5,427,935. Numerous references in the art have shown the preparation of encapsulated cells that may be used to encapsulate the cells herein, see e.g., PCT International patent applications WO 92/19195 or WO 95/05452, incorporated by reference; or U.S. Pat. Nos. 5,639,275; 5,653,975; 4,892,538; 5,156,844; 5,283,187; or U.S. Pat. No. 5,550,050, incorporated by reference. Amino acid polymers that may be used to encapsulate CRL 5803 cells in alginate include the cationic amino acid polymers of lysine, arginine, and mixtures thereof.

Synthetic or natural materials intended for use in the microencapsulated devices must be biocompatible. Preferably, the material should elicit detrimental/harmful response, neither a specific humoral nor cellular immune response, nor a nonspecific foreign body response. A “biocompatible material” is a material that, after implantation in a host, does not elicit a detrimental host response sufficient to result in the rejection of the capsule or to render it inoperable, for example through degradation. The biocompatible material is relatively impermeable to large molecules, such as components of the host's immune system, but is permeable to small molecules, such as insulin, growth factors, and nutrients, while allowing metabolic waste to be removed.

Microencapsulated cells are easily propagated in stirred tank reactors and, with beads sizes in the range of 150-1500 μm in diameter, are easily retained in a perfused reactor using a fine-meshed screen. The ratio of capsule volume to total media volume can be maintained from as dense as 1:2 to 1:10. With intracapsular cell densities of up to 10⁸, the effective cell density in the culture is 1-5×10⁷.

In one aspect, the CRL 5803 cells in the devices provide for the prolonged production of the therapeutic agent in vivo. In particular embodiments, the cells survive in the encapsulated device in vivo for at least two weeks, preferably at least one month. Indeed, it has been found that the CRL 5803 cells maintain at least 50% of the original density even at 90 days and up to one year. Hence the implantable devices of the invention provide for a sustained in vivo delivery of the therapeutic agent for at least two weeks and for up to one year post encapsulation and at least maintain densities for one month post-implantation in rabbit or primate CNS.

In particular embodiments, the cells in the implanted device retain a cell density of at least about 50%, at least about 60%, at least about 70%, at least about 80%, at least 85%, at least 90%, at least 95% or more of the original cell density of the cells that were used in the device. Indeed, it has been found that the cells when implanted may grow in the implantation device to fill the void space in the implantation device and continue to produce the therapeutic agent. This is in contrast to ARPE-19 cells which when encapsulated and implanted to the CNS showed a marked drop in the cell density in the device in vivo.

The advantages of microencapsulation over other processes include the protection from the deleterious effects of shear stresses which occur from sparging and agitation, the ability to easily retain beads for the purpose of using perfused systems, scale up is relatively straightforward and the ability to use the beads for implantation. Thus, encapsulated cell therapy can be used to isolate cells from the recipient host's immune system by surrounding the cells with a semipermeable biocompatible material before implantation within the host. The semipermeable nature of the devices used herein permits the biologically active molecule of interest to easily diffuse from the capsule into the surrounding host tissue.

The CRL-5803 cells are encapsulated and achieve a cell density that is near-tissue density. In exemplary embodiments, the cell density of the CRL 5803 cells in the device is preferably between 0.1 and 4×10⁸ cells/cm³, most preferably between 1 and 4×10⁸ cells/cm³. Dosage may be controlled by implanting a fewer or greater number of capsules, or in preferred embodiments, by increasing the length and/or the diameter of the encapsulation device. In preferred embodiments for the intraparenchymal CNS implantation, the devices contain between about 10⁵ to about 10⁷ cells/cm length.

### Transgenes for Use in Preparation of Recombinant CRL 5803 Cells

The CRL 5803 cells can be used to treat a large number of disease states. Indeed, any disease state that may be treated by the administration of various therapeutic polynucleotides, which may encode polypeptides including tumor suppressors, lymphokines, interferons, growth factors, tissue plasminogen activator, insulin, erythropoietin, thymidine kinase, and the like may be treated using the CRL 5803 cell-based delivery devices of the invention. Moreover, the selective delivery of therapeutic polynucleotides encoding toxic peptides to diseased, hyperplastic, or neoplastic cells can have major therapeutic benefits. The tremendous promise of conventional gene therapy is potentially limited due to a number of factors including inefficiency of gene transfer and limited DNA or RNA capacity of viruses or other vectors. Additionally, gene therapy vectors can be difficult to prepare and purify in large quantities and the cells of the invention can deliver the requisite protein or therapeutic agent to the desired site by simply implanting the device at that site.

The CRL 5803 cells of the invention may be used to deliver various nucleic acids encoding therapeutic or prophylactic nucleic acids into or in the proximity of a target cell in vivo. In certain embodiments, the nucleic acids include one or more therapeutic or prophylactic polynucleotides. Various polynucleotides encode a functional protein, polypeptide, or peptide. “Therapeutic polynucleotide” is a polynucleotide that can be administered to a subject for treating or ameliorating a disease. “Prophylactic polynucleotide” is a polynucleotide that can be administered to a subject for preventing a disease.

The therapeutic or prophylactic polynucleotide may be a tumor suppressor gene (or nucleic acid encoding a tumor suppressor), a pro-apoptotic gene, or a gene encoding a hormone, an antibody, or an enzyme. Examples of therapeutic and prophylactic genes include, but are not limited to Rb, CFTR, p16, p21, p27, p57, p73, C-CAM, APC, CTS-1, zac1, scFV ras, DCC, NF-1, NF-2, WT-1, MEN-I, MEN-II, BRCA1, VHL, MMAC1, FCC, MCC, BRCA2, IL-1, IL-2, IL-3, IL-4, IL-5, IL-6, IL-7, IL-8, IL-9, IL-10, IL-11 IL-12, GM-CSF, G-CSF, thymidine kinase, mda7, fus, interferon α, interferon β, interferon γ, ADP, p53, ABLI, BLC1, BLC6, CBFA1, CBL, CSFIR, ERBA, ERBB, EBRB2, ETS1, ETS2, ETV6, FGR, FOX, FYN, HCR, HRAS, JUN, KRAS, LCK, LYN, MDM2, MLL, MYB, MYC, MYCL1, MYCN, NRAS, PIM1, PML, RET, SRC, TAL1, TCL3, YES, MADH4, RB1, TP53, WT1, TNF, BDNF, CNTF, NGF, GDNF, MANF1, MANF2 (CDNF), IGF, GMF, aFGF, bFGF, NT3, NT5, ApoAI, ApoAIV, ApoE, Rap1A, cytosine deaminase, Fab, ScFv, BRCA2, zac1, ATM, HIC-1, DPC-4, FHIT, PTEN, ING1, NOEY1, NOEY2, OVCA1, MADR2, 53BP2, IRF-1, Rb, zac1, DBCCR-1, rks-3, COX-1, TFPI, PGS, Dp, E2F, ras, myc, neu, raf, erb, fms, trk, ret, gsp, hst, ab1, E1A, p300, VEGF, FGF, osteocalcin, thrombospondin, BAI-1, GDAIF, and MCC.

Other examples of therapeutic or prophylactic genes include genes encoding enzymes, which include, but are not limited to, alkaline phosphatase, cytosine deaminase, ACP desaturase, ACP hydroxylase, ADP-glucose pyrophorylase, ATPase, alcohol dehydrogenase, amylase, amyloglucosidase, catalase, cellulase, cyclooxygenase, decarboxylase, dextrinase, esterase, DNA polymerase, RNA polymerase, hyaluron synthase, glucose oxidase, GTPase, helicase, hemicellulase, hyaluronidase, integrase, invertase, isomerase, kinase, lactase, lipase, lipoxygenase, lyase, L-iduronidase, Iduronate-2-sulfatase, Heparan-N-sulfatase, α-N-Acetylglucosaminidase, AcetylCoA:N-acetyltransferase, N-Acetylglucosamine 6-sulfatase, Galactose 6-sulfatase, β-Galactosidase, N-Acetylgalactosamine 4-sulfatase, β-Glucuronidase, hyaluronoglucosaminidase, Aspartylglucosaminidase, Acid lipase, Cystine transporter, Lamp-2, α-Galactosidase A, Acid ceramidase, α-L-Fucosidase, Protective protein, Glucocerebrosidase (β-glucosidase), Galactocerebrosidase, α-Glucosidase, β-Galactosidase, β-Hexosaminidase A, α-D-Mannosidase, β-D-Mannosidase, Arylsulfatase A, Saposin B, Neuraminidase, Phosphotransferase, Phosphotransferase γ-subunit, Multiple sulfatases, Palmitoyl protein thioesterase, Tripeptidyl peptidase I, Acid sphingomyelinase, Cathepsin K, α-Galactosidase B, sialic acid transporter, carbamoyl synthetase I, ornithine transcarbamylase, arginosuccinate synthetase, arginosuccinate lyase, arginase, fumarylacetoacetate hydrolase, phenylalanine hydroxylase, alpha-1 antitrypsin, glucose-6-phosphatase, low-density-lipoprotein receptor, porphobilinogen deaminase, factor VIII, factor IX, cystathione beta.-synthase, branched chain ketoacid decarboxylase, albumin, isovaleryl-CoA dehydrogenase, propionyl CoA carboxylase, methyl malonyl CoA mutase, glutaryl CoA dehydrogenase, pyruvate carboxylase, hepatic phosphorylase, phosphorylase kinase, glycine decarboxylase, Menkes disease copper-transporting ATPase, Wilson's disease copper-transporting ATPase, hypoxanthine-guanine phosphoribosyltransferase, galactose-1-phosphate uridyltransferase, glucose-6-phosphate dehydrogenase, HSV thymidine kinase, cytosine deaminase, and human thymidine kinase.

Further examples of therapeutic or prophylactic polynucleotides include the nucleic acids encoding carbamoyl synthetase I, ornithine transcarbamylase, arginosuccinate synthetase, arginosuccinate lyase, arginase, fumarylacetoacetate hydrolase, phenylalanine hydroxylase, alpha-1 antitrypsin, glucose-6-phosphatase, low-density-lipoprotein receptor, porphobilinogen deaminase, factor VIII, factor IX, cystathione beta.-synthase, branched chain ketoacid decarboxylase, albumin, isovaleryl-CoA dehydrogenase, propionyl CoA carboxylase, methyl malonyl CoA mutase, glutaryl CoA dehydrogenase, insulin, beta.-glucosidase, pyruvate carboxylase, hepatic phosphorylase, phosphorylase kinase, glycine decarboxylase, H-protein, T-protein, Menkes disease copper-transporting ATPase, Wilson's disease copper-transporting ATPase, cytosine deaminase, hypoxanthine-guanine phosphoribosyltransferase, galactose-1-phosphate uridyltransferase, phenylalanine hydroxylase, glucocerbrosidase, sphingomyelinase, α-L-iduronidase, glucose-6-phosphate dehydrogenase, HSV thymidine kinase, and human thymidine kinase.

Therapeutic polynucleotides also include genes encoding hormones, including but not limited to insulin, calcitonin, β-calcitonin gene related peptide, calcitonin gene related peptide, leptin, leptin derived short peptide (OB-3), osteocalcin, hGH, human parathyroid hormone (PTH), parathyroid hormone related peptide, thyroid-stimulating hormone, melatonin, Glucagon-like peptide-1, exendin-4 and other exendins, GiP, glucagon, lipotropins, ACTH, C-peptide of insulin, GHRH and analogs (GnRHa), growth hormone, prolactin, growth hormone releasing hormone, oxytocin, corticotropin releasing hormone (CRH), atrial natriuretic peptide (ANP), thyroxine releasing hormone (TRHrh), follicle stimulating hormone (FSH), placental lactogen, Luteinizing hormone (LH), chorionic gonadotropin, adrenocorticotropin, angiotensin I, angiotensin II, β-endorphin, α-melanocyte stimulating hormone, β-melanocyte stimulating hormone, cholecystokinin, endothelin I, galanin, gastric inhibitory peptide, neurophysins, somatostatin, pancreastatin, pancreatic peptide, peptide YY, PHM, secretin, vasoactive intestinal peptide, vasopressin, vasotocin, enkephalinamide, metorphinamide, amylin, amyloid P component, neuropeptide Y, substance K, substance P, or thyrotropin releasing hormone.

The therapeutic polynucleotides may encode a growth factor such as, for example, a growth factor is selected from the group consisting of brain derived neurotrophic factor (BDNF), neurotrophin-4 (NT-4), ciliary neurotrophic factor (CNTF), Axokine, bFGF, IGF I, IGF II, TGFβ II, Midkine, IL-1β, nerve growth factor (NGF), IL-2/3, IL-6, neurturin (NTN), Neublastin, VEGF, glial cell derived neurotrophic factor (GDNF), mesencephalic astrocyte-derived neurotrophic factor (MANF) 1 and 2, platelet derived growth factor (PDGF), lens epithelium derived growth factor (LEDGF), and pigment epithelium derived growth factor (PEDF).

Any of the polynucleotides encoding one or more of the above-mentioned agents or indeed any other gene whose expression product is desired at a given site in vivo, may be used to prepare recombinant CRL 5803 cells as described above. Such recombinant cells would then be implanted in vivo in encapsulated form.

### Implantation of Devices Containing Recombinant CRL 5803 Cells

In some embodiments of the invention, the device is encapsulated in a capsule that minimizes the deleterious effects of the host's immune system on the CRL 5803 cells in the core of the device. The cells are enclosed within implantable polymeric capsules formed by a microporous membrane as described above. Use of such a technique allows a cell of the invention to deliver a therapeutic polypeptide while reducing the risk of rejection of the cells by the host. The encapsulated devices may be implanted into the host with or without the combined use of immunosuppressive drugs, such as cyclosporin A, rapamycin, FK506, etc. CSA which may be used in the practice of the invention is commercially available under the name SANDIMMUNE® from Sandoz Pharmaceuticals Corporation. FK506, also known as tacrolimus, is commercially available under the trade name PROGRAF® from Fujisawa Healthcare. Rapamycin, also known as sirolimus is commercially available under the trade name RAPAMUNE® from Wyeth-Ayerst Pharmaceuticals Inc. Use of immunophilins as immunosuppressants is conventionally practiced. See, e.g., Goodman and Gilman's The Pharmacological Basis of Therapeutics, 7th Ed, 1985, p. 1299.

The encapsulated cell-based devices are implanted according to known techniques. Many implantation sites are contemplated for the devices and methods of this invention. These implantation sites include, but are not limited to, the central nervous system, including the brain, spinal cord (see, U.S. Pat. Nos. 5,106,627, 5,156,844, and 5,554,148, incorporated by reference), and the aqueous and vitreous humors of the eye (see, PCT International patent application WO 97/34586, incorporated by reference). Aebischer et al. (1991), describes the long term transplantation of a polymer encapsulated dopamine secreting cell (Exper. Neurol., 111:269-275). Tresco et al. (1992) teach polymer encapsulation of neurotransmitter secreting cells (ASAIO, 38:17-23). Winn et al. (1991) shows implantation and behavioural studies in animals after intrastriatal implantation of microencapsulated PC12 cells (Exper. Neurol., 113:322-329).

When implanted in a recipient, the typical biological response by the recipient to the implantable therapeutic device is to form a fibrotic capsule around the device. For cell encapsulation devices, a fibrotic capsule encasing the device most often deprives the encapsulated cells of life sustaining exchange of nutrients and waste products with tissues of a recipient. The result is usually fatal to the encapsulated cells.

When the device is implanted vascular tissues of the recipient can be stimulated to grow into direct, or near direct, contact with the device. This is desirable because the therapeutic product of the device can then be delivered directly to the circulation of the recipient through the vascular tissues that are in contact with the device. Given that the devices of the invention have the ability to maintain long-term production of the therapeutic agent, it will be desirable to allow the vascular tissue to grow near the device.

In the event that the cells in the device need to be replaced, those of skill in the art are aware of methods of retrieving and replacing the cells contained in such devices. See e.g., U.S. Pat. No. 5,387,237 which describes a cell encapsulation device that has at least one opening into the device through which cells can be introduced and removed. Cells are introduced and removed in this, and other similar devices, as a suspension or slurry.

Retrievable cell encapsulation envelopes for use in implantable devices also are known and have been used as artificial endocrine glands see e.g., in U.S. Pat. No. 4,378,016, which describes a housing made of an impermeable hollow stem and a permselective membrane sack. Such a device is surgically implanted in the recipient such that a portion of the stem protrudes from the recipient and the sack portion is placed within the peritoneal cavity surrounded in peritoneal fluid. The sack allows hormones, nutrients, oxygen, and waste products to flow in and out of the sack while preventing bacteria from entering the patient. Upon implantation of the device in a patient, the cells contained therein produce the therapeutic agent in the desired amount. Other implantable devices are described in e.g., U.S. Pat. No. 5,314,471;

U.S. Pat. No. 6,471,687 addresses the need for an implantable containment apparatus made of selectively permeable polymeric material that allows a cell encapsulation device to be placed and replaced in a recipient without damaging or disturbing tissues associated with the selectively permeable polymeric material would be useful. Such an apparatus becomes closely associated with vascular structures without the need to supply angiogenic factors to induce the close vascularization would also be useful. The placement of the devices near to the vascular structures may be desirable so that the therapeutic agent can readily enter the circulation of the recipient.

PCT/US94/07190 discloses a cell encapsulation device that is generally cylindrical in geometry with a flexible cell displacing core enclosed in a selectively permeable membrane. Such devices also may be useful in the present invention.

The device may be implanted into any in vivo site that is in need of the particular therapeutic agent that is being produced by the CRL 5803 cells. As such, the device containing the cells may be implanted into or near a site for the production of a hormone e.g., the thyroid, the adrenal gland, in or near the ovaries, the CNS. Alternatively, the device may be implanted in the pancreas, the lungs, the peritoneal cavity, or any tissue site in need of the therapeutic agent. In some embodiments, where the therapeutic product being delivered is an anti-cancer agent, the site of delivery may be the site of a tumor, for example at or near the tumor, or the cavity left after the tumor has been resected.

It is contemplated that the device can be delivered in combination with other therapies including other therapies for treatment of the particular disorder as well as therapies used to facilitate implantation of the device (e.g., therapies that enhance vascularization at or near the site of implantation) as well as therapies that would prevent rejection of the device (e.g., administration of an immunosuppressant) and therapies that would prevent infection (e.g., antibacterial and/or antiviral agents). Such other therapies may be delivered locally at the site of implantation or may be administered systemically.

### EXAMPLES

In the present Examples exemplary teachings are provided of methods and compositions for the production of CRL-5803 cell lines for use in encapsulated devices for delivery of therapeutic agents.

### Example 1

### Screening for Neuroendocrine Cell Lines

There are numerous human cell lines available that could in theory be used for cell-based delivery. The present invention is directed to the use of CRL-5803 cells and the present Examples provide exemplary teachings for how this cell line was selected and provides the characteristics of this cell line that make it uniquely suited for use in vivo in encapsulated devices. The initial selection studies focused on characterized and readily available cell lines that also would be likely to meet specific regulatory requirements. International repositories of cells represent some of the most readily available, as well as some of the more carefully maintained cell lines. The American Type Culture Collection was largely used for cells supplied in these studies, although a few lines from other sources were also considered.

In the screening process it was decided that the cell line sought should be a neuroendocrine cell as these cells have the requisite cellular machinery that can support high levels of synthesis, processing, and secretion of peptides. Initially the screening efforts were focused on 21 neuroendocrine cell lines (Table 1) and for comparison 5 other lines from such diverse tissues as liver (HepG2/HB8065), kidney (293/CRL1573), lung (A549/CCL185), connective tissue (HT1080), and bladder (HTB-9).

TABLE 1
Cell Lines

| Designation | | |
|---|---|---|
| ATCC (other) | Tissue of Origin | Culture Property |
| CCL251 (TransR) | Colon | Suspension |
| CRL1803 (DrugR & OuaR) | Thyroid | Adherent |
| CRL5816 (Glucose) | Lung | Adherent |
| CRL5803 | Lung | Adherent |
| CRL5808 (DrugR & OuaR) | Lung | Suspension |
| CRL5815 (DrugR) | Lung | Adherent |
| CRL5804 (ExpressL) | Lung | Suspension |
| CRL5867 (Hyg & Oua) | Lung | Suspension |
| CRL5838 (ExpressL) | Lung | Suspension |
| CCL185 Control | Lung | Adherent |
| HTB9 Control | Bladder | Adherent |
| (CM) (MYCO) | Pancreas | Adherent |
| CRL2139 (NE) | Neuroectodermal | Adherent |
| CCL249 (growth, serum) | Colon | Suspension |
| CCL253 (NE) | Cecum | Suspension |
| CRL5893 (TransfectR) | Lung | Suspension |
| CRL5974 (NE) | Stomach | Suspension |
| HTB10 (ExpressL) | Brain | Adherent |
| HTB184 (TransfectR) | Lung | Suspension & Adherent |
| HTB177 (PC) | Lung | Adherent |
| CRL2195 (TransfectR) | Lung | Mostly Suspension |
| (H2098) (ExpressL) | Pancreas | Adherent |
| (BON) (5FU-R) | Pancreas | Adherent |
| CCL121 (serum) | Connective | Adherent |
| | Tissue | |
| CRL1573 Control | Kidney | Adherent |
| HB8065 Control | Liver | Adherent |

Text in parentheses indicates screen step in which the cell line was considered to be disadvantageous with respect to selection requirements.
5FU-R - resistance to 5-fluorouracil;
DrugR - resistance to selection drug(s);
ExpressL - poor transgene expression levels;
Glucose - glucose-dependence;
Growth - long doubling times;
MYCO - mycoplasma contamination;
NE - lack of neuroendocrine markers;
OuabainR - resistance to ouabain;
PC - no detectable prohormone convertase protein;
Serum - serum-dependence;
TransR - transfection resistance as gauged by transgene expression.

Four of the neuroendocrine cells were from the gut, eleven were from the lung, three were from the pancreas, one from the thyroid and one of neuroectodermal origin. All except three of the cell lines were obtained from the American Type Culture Collection (ATCC), CM (Cavallo; Monetini) was obtained from P. Pozzilli (University of Rome La Sapienza), BON (Evers et al. 1991 Gastroenterology 101:303-311; Townshend et al. 1993, Acta Oncol. 32:125-130) was obtained from C. M. Townshend, Jr. (University of Texas Medical Branch, Galveston) and H2098 was obtained from A. Gazder (University of Texas Southweatern Medical School). Cell Culture, growth and cytotoxicity assays were monitored using standard assays as described below.

A. Measuring Viable Cell Mass

In order to measure viable cell mass, a resazurin-based assay was used. The non-fluorescent molecule resazurin (Sigma-Aldrich Chemicals, Tres Cantos, Madrid) was used to follow the functional mass of cultured cells. Cellular enzymes convert resazurin to the fluorescent metabolite-resorufin. The rate of resorufin formation is a linear function of viable cell number, and of cellular metabolism (metabolism was quantified by measuring overnight glucose consumption and lactate formation with human cells, r2=0.98, 0.99 for correlation of glucose and lactate, respectively, with resazurin metabolism).

Resazurin is made as an 80 μM stock solution in PBS (Ca and Mg-free), which is then mixed 1:1 with culture medium for assay of cellular activity. With cells grown in multiwell plates, the rate of the reaction is followed for 15 minutes (reads at 30s intervals) to provide enough data points to calculate reliable rates. This short assay reaction has no measurably deleterious impact on cells, for this reason the same cultures can be followed continuously with the resazurin assay. This assay not only offers the capacity to determine cell number from standard curves of resazurin activity, but also, with the repeated measurement of cellular activity over time, it provides a means to reliably determine growth rates and doubling times.

B. Cell Culture and Elimination of CCL249 and CCL121

In order to facilitate comparibility, all cells were adapted to culture in a 1:1 mixture of DME and F12 medium, at a final glucose concentration of 7.8 mM (JRH Biosciences, Lenexa Kans.). The medium was supplemented with Australian-sourced, gamma-irradiated fetal bovine serum (FBS; JRH Biosciences) at levels of 1-3%. The serum was pre-screened (by comparing growth in varied percentages of FBS) before purchase to ensure that an indicator cell line could be grown effectively with 0.5% serum-supplementation.

The CNS environment has a greatly reduced complement of serum-related proteins. It is for this reason an early selection was for cells that have reduced serum requirements for growth. Each cell line was examined for the individual growth requirements and supplemented with lower (1-3%) FBS accordingly. An inability of cells to adapt to reduced serum was used as an initial exclusion screen. One cell line (CCL249) was not studied further due to difficulties in growing sufficient numbers of cells prior to reducing the serum, and it was reasoned that growth characteristics that severely limit bench-scale cultures could preclude commercial manufacture. Another cell line (CCL121) was not studied further because the growth properties of this cell did not readily provide for low serum culture. All other cell lines were adapted to culture with medium supplemented with lower serum.

Each cell line was screened for mycoplasma expression, both by a PCR assay (ATCC) and a plate culture method (Mycotrim, Irvine Scientific, Santa Ana, Calif.). The CM cell line was eliminated in early cultures due to mycoplasma infection.

C. Screening for Protein Expression by Western Analysis and Elimination of HTB177, CRL5974, CCL253, CRL2139

Further selection of these cell lines was accomplished by analyzing the expression of the following neuroendocrine markers-synaptophysin, chromagranin A, prohormone convertase enzymes ⅓ and 2 (PC1 and PC2), carboxypeptidase E (CPE), as well as PAM, the enzyme for alpha amidation. These proteins were identified by Western-blotting with extracts of cells from each of these cell lines.

Late log phase to plateau phase cultures of the different cell lines grown in 3-5% FBS were harvested with dilute trypsin-EDTA (0.05%, 0.02%), pelleted by centrifugation (≈500×g) and washed at least 3× with PBS to remove the trypsin and the final pellet was resuspended in a buffer (20 mM K₂HPO₄, 1 mM EDTA, 110 mM KCl, 0.5% triton X100, pH 8.0, and proteolytic inhibitors) for sonication on ice with 3 bursts of 3-5 sec at 30% power, and for most analyses cleared sonicate (14,000-18,000×g, 5 minutes) was analyzed. Protein content of the sonicate was measured using a Bradford protein analysis kit (BioRad, Hercules Calif.). The concentration of protein was adjusted so that equal amounts of protein were used for each cell line. The sonicate was mixed with 2× sample buffer, heated at 90° C. , cooled, then loaded into precast gels for protein separation and transfer for subsequent antibody-based detection of specific proteins. Gels, buffers and markers were obtained from Novex (Invitrogen). Antibodies were as follows: synaptophysin (Boehringer Mannheim), chromagranin A/pancreastatin (Biogenesis, Kingston, N.H.), PC1 (Dr. D. Steiner, Univ. Chicago), PC2 (Dr. C. Rhodes, Univ. Texas Southwestern), CPE (BD-Transduction Laboratories, San Jose, Calif.), PAM (Dr. B. A. Eipper, Johns Hopkins University). Detection was with alkaline phosphatase-labeled secondary antibodies, visualized with nitroblue tetrazolium-bromochloroindoyl-phosphate (NBT-BCIP) substrate-dye solution in Tris buffer (pH 9).

As it is possible for cells to produce detectable RNA for a gene and yet fail to produce detectable expression of the related protein, Western analysis was used to directly evaluate expression at the protein level. Additionally, a public database for RNA expression in cell lines was examined, data for the cell line HTB177 expression was reported (http://dtp.nci.nih.gov/mtweb/targetdata) for a large number of genes. The results of 3 different analyses suggested that the PC1 gene was well expressed in HTB177 cells, with 3 to 10 times greater signal than average, and than CCL185 cells. In contrast, in the western analyses there was no detectable PC1 protein in HTB177 cells, and very low expression in CCL185 cells. In 12 other cell lines of the panel, immunoreactive PC1 was readily detected. These results indicate a discordance of RNA- and protein-expression characteristics, at a minimum, for the CCL185 and HTB177 cell lines.

In the selection criteria, a cell line was considered to have a neuroendocrine (NE) profile when 1) either of the two proconvertases, 2) either CPE or PAM, and 3) either synaptophysin or chromagranin, were expressed. Four cell lines (HTB177, CRL5974, CCL253, CRL2139) were eliminated due to no or relatively poor expression of neuroendocrine markers. These four were not readily distinguishable from the non-endocrine cell lines CRL1573/293, HTB9, and HB8065/HepG2 in terms of neuroendocrine markers.

### Example 2

### Transfectability of Selected Neuroendocrine Cells

The 15 NE cell lines remaining from the studies performed in Example 1 were then screened for ability to be transfected. First, this involved testing for resistance/sensitivity to selection drugs G418, and hygromycin, along with sensitivity to 5-fluorouracil, or ouabain. Secondly, cells were tested for the ability to express a transfected gene, such as neomycin phosphotransferase (NPT), growth hormone (hGH), glucagon, and insulin. If the cells are not sensitive to killing by selection drugs (at least 50% killing at concentrations ≦1 mg/ml) it would make isolation of an expressing clone impractical. The test for 5-FU killing was used to ensure sensitivity to a chemotherapeutic and to indicate the feasibility of cell suicide systems (5-FU is the cytotoxic active molecule for the pro-drug 5-fluorocytosine). Human cells are quite sensitive to killing by ouabain, typically 10-100 times more sensitive than rodent cells, thus resistance to ouabain indicates a concern in terms of either a general drug-resistant cell, or human cells that may be contaminated with cells of another species. Expression of an exogenous gene screens for the combined ability of the cells to be transfected, and the relative level of protein expression for a transgene. As the brain environment has a lower glucose concentration than the periphery, a final stage screen was used to test for glucose-dependence of cell growth.

A. Screening for Sensitivity to Chemotherapeutic-5 Fluorouracil

An advantage of “encapsulated-cell therapy” is an element of safety provided by containment of the cells inside of a cell-encapsulating device. This physical barrier ensures that the cells remain at the site of implantation and limits the cellular mass, preventing unwanted proliferation, as well as migration to other regions of the body. However, careful safety assessment considers the impact of unexpected occurrences, such as device failure due to trauma, implant handling, manufacturing defects, or other causes. Should such a failure occur, one desires to be prepared to maintain control of the implanted cells in all cases of failure, no matter how improbable.

Some cells are quite resistant to chemotherapeutics, such as normal hepatocytes, or many tumor cells. Should a cell line have characteristics that provide for resistance, whether enzymatic inactivation, transport, repair or other means, it would be a property that one should consider in cell selection. Whether that consideration leads to excluding the cell as a candidate, or leads one to design a cell suicide system that will provide a means for effective elimination of the cell.

One well known chemotherapeutic is 5-fluorouracil (5-FU). This drug was selected to use for evaluating resistance of cells. Cells that overexpress multi-drug resistance (MDR) transporters, or those that express the enzyme dipyrimidine dehydrogenase (DPD) can overcome the toxic action of 5-FU. Further, this drug was selected for use in resistance screening because 5-FU is the effector molecule for a common cell suicide system involving the enzyme cytosine deaminase (CD). This cell suicide system confers toxicity of the prodrug 5-fluorocytosine (5FC) to a cell when cytosine deaminase is expressed. Cytosine deaminase metabolizes the normally nontoxic (for human cells) 5-FC to the chemotherapeutic toxin 5-FU. This system has been shown to be effective in the CNS, not blocked by the blood-brain barrier (Miller, 2002, Cancer Res 62:773-780). There are molecular screens to indicate sensitivity, actual cytotoxicity testing of 5FU was used to more closely reflect the capabilities of a CD-5FC system (Grem, 2001, Clin Cancer Res 7:999-1009; Miller 2002, Cancer Res 62:773-780).

Drug Exposure and elimination of BON cell line: The drug 5-FU was dissolved in DMSO at a concentration of 0.5 M, and this stock used to make media with micromolar concentrations of the drug (5-500 VM). Cells plated in 24 or 48 well plates in growth phase were cultured with 5-FU supplemented medium for 1-2 weeks. Growth was determined in the 1-2 week period by repeatedly measuring viable cell mass with time, the data indicate cell survival and are used to calculate the growth (doublings) of the population. This growth measurement was conducted during exposure and if cells grew or survived, growth was followed in the weeks subsequent to removal of 5-FU.

In initial screening, an endpoint assay of viability was performed with the uptake of neutral red (50 μg/ml for 1 h) after 5-7 days, later studies of the selected cell lines used resazurin to monitor growth, as well as viable cell mass.

Based on the cytotoxicity of 5-FU the BON cell line was excluded because the concentration required to kill 50% of the cells with 5-FU appeared to be ≧500 μM

B. Screening for Sensitivity to Selection Drug and Elimination of CRL1803, CRL5808, CRL5815 and CRL5867

The cell lines were plated in 48 well plates at an appropriate dilution of cells for each cell line to provide for rapid entry into log phase of growth. The various concentrations of the selection drugs were added so that the drugs were present at the initiation of log phase growth and viable cells were determined after 7-10 days of culture. The screening used data for functional mass at the end of the study to express viable cells as a percentage of the vehicle, or no-treatment control wells. Each treatment concentration was based on results for at least 3-4 wells. The % viable cell mass data was plotted, and fitted typically with a four-parameter equation. The fitted equation was used to calculate the IC50 dose. The drugs tested were G418 (inactivated by neomycin phophotransferase, NPT), hygromycin (inactivated by hygromycin phosphotansferse, HPT), blasticidin (inactivated by blasticidin deaminase, BSD), and zeocin (bound by bleomycin binding protein, BBP). These antibiotics were obtained from Invitrogen/Gibco (Invitrogen SA, Barcelona). Cells were considered to be resistant if the IC50 was >1000 μg/ml for G418, hygromycin, and zeocin, or concentrations >20 μg/ml for blasticidin. Although ouabain is not a classical selection drug, the rodent gene for Na—K ATPase shifts the sensitivity into the 10⁻⁴ M range, while human cells are sensitive in the 10⁻⁹ to 10⁻⁶ M range, thus IC50s in the 10⁻⁴ M range were taken to indicate resistance.

Four cell lines were excluded with the selection drug screen. Two cell lines, CRL1803 and CRL5808, were eliminated, because these lines were resistant both to selection drugs and to ouabain. The cell line CRL5815 was resistant to selection drugs, and was excluded. One cell line, CRL5867, was sensitive to killing by G418, but was excluded because of resistance to both hygromycin and ouabain.

An unintended outcome of the selection drug screen was the observation that individual cell lines can be characterized in terms of dose-sensitivity to toxic action of given set of different compounds. The differential toxicity of each cell line is a characteristic that can be used to define the line and to assess the stability of the toxicity characteristics. Such a shift in toxicity characteristics would be an indication of an unstable population of cells, although it would not indicate the cause of instability. These toxicity characteristics can be used to advantage to ensure purity of a cell population, such as when preparing samples for DNA array analyses to characterize cellular expression profiles, or DNA fingerprinting.

### Example 3

### Screen for Transfection by Expression of Resistance Gene

Transgenic engineering of cells is most effective when the engineered cells can be isolated to provide for appropriate enrichment of the cells expressing the transfected transgene. This isolation is most conveniently effected by expression of the transgene for the desired protein in combination with a gene that confers antibiotic resistance. In order for the conferred resistance gene to provide for effective isolation of expressing cells, the cells used need to have at least two distinct properties: 1) the unengineered cells need to be appropriately sensitive to the cytotoxic action of the antibiotic, and 2) the expression of the resistance gene needs to be high enough to shift the cytotoxic threshold to a concentration readily distinguished from non-engineered cells. The latter shift in cytotoxic threshold involves both transcriptional, translational, and potentially post-translational events.

A. Plasmids and Selection Markers and Elimination of CCL251, CRL5893, HTB184, CRL 2195, CRL5804, CRL5838, HTB10, and H2058

Plasmids containing the blasticidin-S-deaminase gene or the bleomycin binding-protein were obtained from Invitrogen (Barcelona). Neomycin-phosphotransferase and hygromycin phosphotransferase genes were used in the vector-backbones as previously described (Clark et al., Diabetes 46:958-967, 1997).

The 10 cell lines remaining after previous screens were examined in terms of transfectability. Four cell lines, CCL251, CRL5893, HTB184, and CRL 2195, were not successfully transfected with plasmids that used an SV40-driven neomycin phosphotransferase (NPT) resistance marker, in that these cells did not form G418-resistant colonies when grown under this selection. The inability to grow colonies was either due to lack of sufficient NPT expression, or to very poor cloning efficiency, or poor transfection efficiency, or combinations of these factors.

With the remaining 6 cell lines, G418-resistance was conferred. Only 2 cell lines consistently provided robust expression with different transgenes (GH, cytosine deaminase, GLUT2) driven by the CMV promoter. Thus, with this screen the cell lines CRL5804, CRL5838, HTB10, and H2058 were not considered robust in terms of transgene expression and were not continued as candidates for expression of proteins in the CNS. The two cell lines that emerged from these various screens as candidates for further consideration were the lines CRL5816, and CRL 5803.

It should be noted that in subsequent studies (discussed below) it was shown that SV40 and CMV promoters do not provide the best expression with all human cell lines, thus some of the human cell lines that were excluded in the present screen could be considered in the context of other promoters.

B. Screen for Growth/Survival at Low Glucose and Elimination of CRL5816 from Screen

An additional consideration in selecting cells for use in the CNS, is that the glucose concentration in the CSF is lower than in the blood. The concentration of glucose in the CSF averages about 3 mM, or 66% of the serum concentration (IHenry, Clinical Diagnosis and Management by Laboratory Methods, 2001, 20th edition, W. B. Saunders) as a result cells used in the CNS should exhibit the capacity to survive and function at glucose concentrations >3 mM.

Varied glucose concentrations were obtained by mixing medium custom-made without glucose (but all other nutrients were normal) with the same medium containing 7.8 mM glucose, or additional glucose from a stock of 50% glucose. Growth of cells, in log phase through plateau phase, was determined to indicate the effects of glucose on growth.

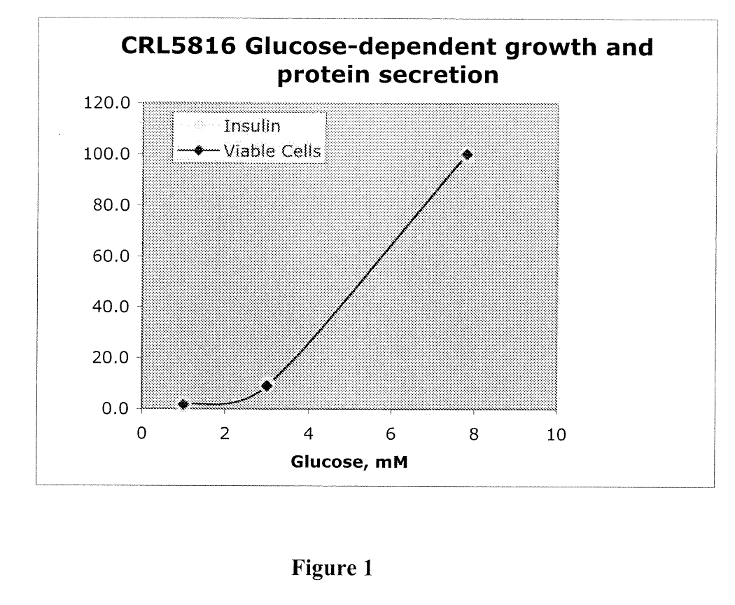

In studies of the nutrient requirements of the NE-cell candidates, it was found that CRL5816 cells exhibited a glucose-dependence for growth and survival in a five day assay (FIG. 1), and expressed no detectable hexokinase I as measured by western blot. The CRL5816 cell line exhibits poor survival and low transgenic protein expression when cultured at 3 mM glucose (FIG. 1). This corresponded to a sensitivity to killing by 2-deoxyglucose: after 4 days-1 and 3 mM glucose resulted in viable cell mass that was 5 and 13% of 7.8 mM glucose control, but 10 mM 2-deoxyglucose yielded no detectable viable cells.

The glucose requirements of CRL 5803 was also determined and compared to the HTB10 and CRL5816 cell lines. The cell line HTB10 also exhibited a high sensitivity to 2-DG (2-DG concentration for 50% killing was 0.6 mM), while the cell line CRL 5803 was neither growth constrained by glucose, nor was it sensitive to killing by 2-deoxyglucose (with up to 20 mM 2-DG). From this data it was inferred that the CRL5816 cell line was not likely to be sufficiently robust to thrive in the context of the CNS environment, and thus, the CRL 5803 was selected as a candidate neuroendocrine cell line for CNS delivery of proteins.

### Example 4

### Comparison of NE- and Other-Cell Lines for Protein Delivery in the CNS

In addition to the NE-cell CRL 5803, other human cell types were selected to test and compare as candidates for CNS delivery. One cell line selected to include in such testing was the retinal pigmented epithelial cell line ARPE-19. This line was chosen because it is recognized as an optimal cell line for delivery to the eye and the CNS (U.S. Pat. No. 6,361,771). In addition, ARPE-19 cells are known to endogenously produce endogenous neurotrophic factors (Kanuga 2002, Invest Ophthalmol Vis Sci 43:546-55). Two other cell types were chosen, normal fibroblasts and melanoma cells, because these cell types have been reported to survive in the CNS environment (Duanet al., Exp Brain Res 161:316-24, 2005), or, because the melanoma line is expected to express neurotrophic factors (Innominato et al., J Pathol 194:95-100, 2001), factors of therapeutic significance for CNS delivery. A fourth cell type, smooth muscle, was selected also because this cell type reportedly expresses neurotrophic factors (Ricci et al., J Vasc Res 37:355-63, 2000). In summary, the cell lines selected were CCL205 a normal human fibroblast, A2058 a melanoma cell line, and CRL1440 a smooth muscle cell line, in addition to ARPE-19 and CRL 5803.

A. Sensitivity to 5-FU

The newly selected cell types were then screened for r sensitivity to 5-fluorouracil (5-FU). This screening was accomplished by plating the cell lines in 48 well plates, and after overnight attachment adding varying concentrations of 5-FU from 0-500 μM. The growth of the cells was then monitored at 1-2 day intervals with the fluorescent substrate resazurin. Exposure to 40 μM resazurin for periods of 1-4 days is cytotoxic with some of these cell types, but none have exhibited deleterious effects with repetetive, short exposures of 15 to 60 minutes. The chronic exposure cytotoxicity appears to be related to the product—resorufin—which was found to be toxic with many cells at 1-10 μM.

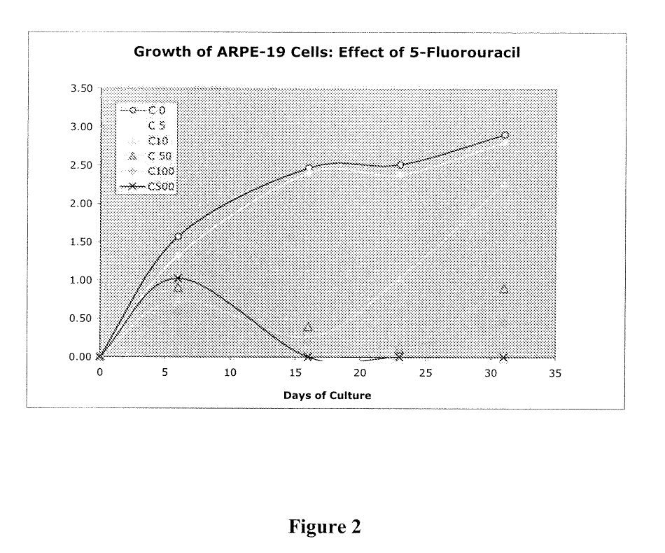

The concentration of 5-FU that resulted in 50% killing was <35 PM for all cell lines except for ARPE-19 and A2058. The cell line A2058 was not affected by <10 μM 5-FU. The response was dominantly cytostatic with concentrations of >10 to 100 μM, and began to kill at >500 μM. Similarly, the effect of 5-FU on ARPE-19 cells was not predominantly killing. In the first week of 5-FU treatment concentrations >5 μM slowed growth, but the cells were not killed by 5-FU exposure. The effect of 5-FU in the second week appeared to involve both killing and growth arrest of ARPE-19 cells at doses >10 μM, and while 100 μM was not a lethal dose, 500 μM was a lethal dose (FIG. 2). However, with 5-FU removal subsequent to 2 weeks of exposure, the growth arrested cells re-initiated growth, with normal doubling times. Growth was even re-initiated after 100 μM 5-FU. Only after exposure to 100 μM 5-FU for two periods of 2 weeks was the doubling time altered, resulting in a prolonged doubling time (60 h in ARPE-19 exposed to 100 VM twice, vs 36 h doubling time for control cells), but even this double exposure to 100 μM regimen was not lethal.

Based on 5-FU sensitivity, a cell suicide gene, cytosine deaminase, would be expected to be effective with the cell lines CRL 5803, CCL-205 and CRL1440.

The resistance to 5-FU was a criterion set for excluding cells as candidates for use in CNS delivery, as this resistance can be used to indicate a safety risk that may not be overcome readily. Therefore, the A2058 cell line was not selected as a candidate, nor would the ARPE-19 cells be used as a reasonable candidate. However, the use of ARPE-19 cells was continued in subsequent testing, not as a “candidate” cell but for comparison purposes. The ARPE-19 cells not only represent a cell line that is claimed in a patent, but also a derivative is in use for therapeutic trials in eye disease (www.neurotech.com).

B. Selectable Markers

The dose sensitivity of four cell lines, CCL205, CRL1440, ARPE-19, and CRL 5803 was determined to ensure that the correct concentrations would be known for subsequent transfections, and that the 3 newer lines were all appropriately sensitive. The drugs, G418, Zeocin, hygromycin, and blasticidin were obtained and used as described earlier. The most important aspect of these studies was to identify the concentration needed to kill all non-transfected cells. The concentration, μg/ml, found to kill non-transfected cells is presented in Table 2.

TABLE 2

| Drug | CRL5803 | ARPE-19 | CRL1440 | CCL205 |
|---|---|---|---|---|
| G418 | 500 | 175 | 100 | 75 |
| Hygromycin | 300 | 100 | 50 | 50 |
| Blasticidin | 2 | 3 | 1 | 1 |
| Zeocin | 500 | 50 | 100 | <20 |

### Example 5

### Nutrient Requirements and the CNS Environment

In vivo environment: Cell culture systems are usually optimized for growth, and thus favor cell replication. In vivo, cell replication is supported, but characteristically replication is favored in only certain niche environments. A cell that is dominantly replicative in vitro may not be so when implanted into certain organ regions in vivo. An example where the local environment influences these properties can be found with lung-tumor cell lines that have different growth properties when implanted in the lung versus other tissues in the body (Onn, Clin Cancer Res 15:5532-39, 2003). One would then predict that functional properties cells exhibit in vitro, in conventional culture, may not mirror functional properties of the same cells in specialized environments of the body.

The central nervous system represents one such specialized environment. Unlike the rest of the body, the CNS predominately consists of two functionally distinct cells-neurons and astroglia. The interconnected fluid-filled regions of the CNS contain constantly evolved cerebrospinal fluid (CSF), CSF presents another unique feature of this specialized environment. Further, the brain environment is protected/isolated from the fluids that are provided to the remainder of the organism by the cardiovascular system. This separation is effected by the specialized nature of the blood vessels permeating the brain. These blood vessels form what is called the blood-brain barrier (BBB), which provides for the selective passage of molecules from the blood and into the brain parenchyma. The end result of this barrier is that the composition of the fluids in the brain are quite unlike the composition of blood or lymph, the fluids that bathe cells of other body regions (Henry, Ed., Clinical Diagnosis and Management by Laboratory Methods 20th Edition, 2001).

A further indication of the unique environment of the CNS is the reaction to implanted materials. Materials that have been used safely in other sites in the body have been found to be deleterious in the context of the CNS. The present application has evaluated the candidate delivery cells in terms of the impact of several of the bulk constituents of blood and brain fluids that represent some of the major compositional differences between these two fluids of the body. The effect of altering these constituents provide a means to identify cells that exhibit the capacity to function in the brain/CNS environment. These main constituents are described below.

Neurons of the brain use specific amino acids in cell-cell communication in a context where the “background” levels of amino acids are less than 10% of the concentrations found in the plasma compartment. Another anabolic constituent of blood-glucose-is also reduced, though not to the degree of amino acids. Glucose in the CSF is 60% of blood values, with much lower values, and regional variations in the intercellular fluids of the brain parenchyma (Levin et al., Diabetes 53:252-8, 2004). The concentration of many blood/plasma proteins is diminished to small percentages in the CSF. Albumin for example is present in plasma at 4-6% levels (weight:volume), and in the brain at less than 0.1%. The concentration of the critical cation calcium is reduced by about half in the CNS (Henry, Ed., 2001 vide supra).

Rather than screen for survival in a CSF-like fluid, (or artificial CSF) which may contain no amino acids or protein, we considered that a more informative approach would be to evaluate the importance of these constituents representing some of the major nutrient differences between CSF and serum. The following bulk constituents were varied in a cell culture system to evaluate the functional impact: 1) varied and up to 100% reductions in serum protein; 2) 90 to almost 100% reductions in amino acids; 3) 50-90% reductions in glucose; 4) 50-90% reductions in extracellular calcium. In these studies growth rates of cells were determined with cells cultured in 48 well plates. Serum-dependency was evaluated by plating cells at varying split ratios from a confluent culture into fetal bovine serum at varied supplementations of 0.3 to 6%. The remaining three constituents were evaluated in dense, plateau-phase cultures. The dependency for these three constituents was evaluated in the context of a simple, physiologic salt solution resembling Kreb's-Ringer bicarbonate solution, consisting of: 114 mM NaCl; 4.7 mM KCl; 1.2 mM KH2PO4; 25.5 mM HCO3; 2.5 mM CaCl2, 7.5 mM glucose, 2.4 mM MgSO4, and 0.8% FBS. This salt, glucose, and serum mixture is referred to as M7 solution.

A. Serum Dependence

In order to emphasize the serum requirement of cells, the cells were plated at very low split ratios of 1:200, 1:400, and 1:1600 (that is, the number of cells from 1 cm² of a confluent monolayer to be plated on 200, 400, or 1600 cm of culture surface), in either 0.3, 1.0, 3.0, or 6.0% FBS. The cells were then monitored for 2 weeks, the cell number was low enough in the first 7 days that cell mass was not readily measured with precision, growth was then evaluated for the second week. With this approach the ability to grow in the combination of high split ratios and low serum indicates the cells with the least serum dependency. Cells with lower serum dependency would be expected to function more optimally in the context of the low serum protein environment of the CNS.

Of the four cell lines examined only CRL 5803 cells grew at all twelve combinations of split ratios and serum. The doubling times for CRL 5803 cells at the 1:1600 split ratio changed minimally from 28 h at 0.3% FBS to 31 h in 6% FBS. Each of the other cell lines exhibited densities at which the cells would not grow in low serum. The CRL1440 cell line did not grow measurably with 2 of the 12 combinations, one of these 2 was due to lack of survival. With ARPE-19 cells, cells did not grow measurably with 4 of the 12 combinations, three of these were due to non-survival. CCL205 cells did not grow measurably with 9 of 12 combinations, due to non-survival in 8 of 12, and with growth only at the 1:200 ratio and >1% FBS . The rank order of serum dependence was CRL 5803<CRL1440<ARPE-19<CCL205. These results indicate that in the CNS environment, in which serum represents a rate-limiting condition, the CRL 5803 cells would be most capable of adapting, while the other cell lines would require optimized conditions to provide survival and functional advantages.

B. Amino Acid Dependence

In order to study amino acid dependence the physioloic salt mixture with glucose supplemented to 7.5 mM, and serum to 0.8% (M7 solution) was used. This M7 solution was then mixed with DMEM/F12 containing 0.8% FBS to provide 2% or 10% (volume/volume %) medium, and thus either 2 or 10% of “normal” amino acids. Established, plateau phase cultures were switched from medium to M7 solution and the impact on viable cell mass was measured after 3 days, each culture was then “rescued” 7 days with 2% and then 7 days with 10% amino acids to determine the “rescue” requirements for each cell line. At least 12 wells were studied for each cell line.

Only the CRL 5803 cell line did not lose viable mass in the 3 day switch to M7, maintaining a confluent monolayer. The other 3 cell lines lost 40-50% of viable mass. The CRL 5803 cell line did lose cell mass with continued culture time in M7 supplemented with 2% amino acids, thus 2% supplementation from medium did not stabilize confluent cells during 7 days of culture. Similarly, 2% amino acids did not stabilize CRL1440 cells, these cells continued cell loss to less than 25% of original mass. Both the ARPE-19 and CCL205 cell lines were subconfluent and were stabilized with 2% supplementation, although 2% was not sufficient to initiate growth. All 4 cell lines grew with 10% medium amino acid supplementation, CCL205 cells only grew in the first 3 days, the other 3 cell lines grew the entire 7 days. Net doublings in 7 days with 10% medium amino acids: 0.5 for ARPE-19, 0.9 for CRL 5803, 1.0 for CCL205 and 1.7 for CRL1440.

In summary, all 4 cell lines grew with 10% amino acid rescue, whereas only ARPE-19 and CCL205 cells stabilized with 2%. These results indicate that all 4 cell lines may encounter functional difficulties with CSF concentrations of amino acids, although subconfluent CRL 5803, ARPE-19, and CCL-205 cells may have a capacity to better withstand the low amino acid concentration.

C. Glucose Dependence of Cells

In studies of the nutrient requirements for the NE-cell candidates we observed that cells that exhibited a glucose-dependence for growth and survival exhibit a corresponding sensitivity to 2-deoxyglucose (see Example 3B above). The 2-deoxyglucose sensitivity was tested with the 4 cell lines being screened for CNS use with doses of 2-DG from 1-20 mM, the HTB10 cell line was also included in this screen as a positive control for 2-DG killing. While the HTB10 cell line was killed with the lowest concentration of 2-DG, there was no killing with any of the four candidate cell lines. However, 2-DG did result in slowed growth, and concentrations producing 50% inhibition (IC50) were calculated (based on a four-parameter fit of the data). The IC50s ranged from 5-11 mM with a concentration sensitivity ranking where the most sensitive cells the CRL1440s≈A2058<CRL 5803<ARPE-19.

To confirm the results of 2-DG sensitivity the growth of the cell lines was measured at glucose concentrations between 0.7 and 5.5 mM. With normal medium neither the ARPE-19 nor the CRL 5803 cell line readily exhibits effects of glucose on growth. Therefore, the effect of glucose when amino acids are limited was examined using M7 solution supplemented with 10% medium.

Most of these cell lines exhibit cell loss when glucose is reduced to 0.7 mM and amino acids are restricted. Inconsistent with the IC50 for 2-DG, CRL1440 cells did not exhibit loss at 0.7 mM glucose, the CCL205 cells decreased 12%, while the ARPE-19 cells had 26% cell loss at 0.7 mM glucose, and the line CRL 5803 exhibited 56% cell loss when switched to 0.7 mM glucose and restricted amino acids. Other than CRL1440 cells, the cell lines grew when the glucose concentration was increased to 2.2 mM for 2 days. The growth of CCL205 and CRL 5803 cells continued with increased glucose to 5.5 mM for 5 days. The growth was reasonably proportional to the loss; CRL1440 cells did not grow with increased glucose. In contrast, ARPE-19, CRL5803, and CCL205 grew modestly with 0.4±0.3, 0.9±0.1, and 0.3±0.2 doublings in 7 days, respectively.

Although CRL1440, ARPE-19, and CCL205 appear to have a lesser absolute glucose dependence under amino acid restricted conditions, than CRL 5803 cells, clearly CRL 5803 cells can grow in glucose concentrations that would be found in the CSF (2.2 mM). This ability to grow at low glucose and restricted amino acids indicated the potential of CRL 5803 for use in cell-based delivery to the CNS.

D. Calcium Dependence

The study of the calcium dependence of cells also used M7 solution, except that in preparing the M7 solution two different calcium formulations were made, one with 2.5 mM calcium and one without. Both M7 solutions were supplemented with 10% medium and 0.8% FBS, these two solutions were mixed with equal volumes of 0 and 2.5 mM calcium to provide a third solution. In this way, 3 different media were made, all the same in terms of other salts, glucose, amino acids, and FBS, but differing in calcium. The lowest had ≈0.25 mM derived from the 10% medium supplement, the intermediate (and closest to CSF concentrations) was ≈1.38 mM, and the highest was ≈2.5 mM calcium. Plateau-phase cultures were first changed into medium with 0.8% FBS for 3 days, then M7 at 2.5 mM calcium for 3 days, subsequently into M7 at 1.38 mM calcium for 2 days and finally M7 at 0.25 mM calcium for 4 days.

Of the 4 cell lines studied, only 2 cells lines exhibited a change in cell mass in medium with 1.25 mM calcium—CCL205 with a slight decrease (20±4%) and CRL1440 with a slight increase (29±16). In contrast, all four cell lines responded to 0.25% calcium, two lines decreased and two increased. CRL 5803 decreased 20±4%, CRL1440 decreased 33±1%, ARPE-19 increased 34±4%, and CCL205 increased 41±5%. Although the data suggest that with ARPE-19 and CCL205 cells, 0.25 mM calcium has an acute stimulatory effect, overall the data do not indicate that any of the 4 cell lines would be negatively impacted by the calcium concentration of CSF. Note that overall, these studies of calcium are in a solution that approximates CSF in that serum is <1%, and amino acids are about 10-fold lower than in serum.

Commercially available artificial CSF contains salts and no proteins, in the patent for ARPE-19 cells a formulation was used that excluded calcium and glucose. In the present set of studies a salt formulation supplemented with glucose, low amino acids, and low FBS was used to approximate CSF. There are limitations with each of these approximations, particularly in that the concentration of any nutrient provided in vitro will decrease with time, while in vivo the nutrient concentrations are sustained by the blood and CSF circulation. Nonetheless, the results in these studies do not provide a means to exclude any of the 4 cell lines, all appear to be capable of survival and some growth under CSF-like conditions. This is in contrast to the screening used in U.S. Pat. No. 6,361,771, in which ARPE-19 was one of the few cell lines found to survive in a CSF-like solution, it is believed that this is related to the omission of calcium and glucose from the ‘CSF-like’ solution used in those experiments.

The present studies were deliberately performed with dense, plateau-phase cultures in an effort to examine the sensitivities of these cell lines in a state that would be similar to dense cultures in an implantible bioreactor device (as described in patents U.S. Pat. Nos. 5,980,889, and 6,426,214, each incorporated herein by reference)

### Example 6

### Transfection for Transgene Expression

A. Optimizing Transgene Expression

Different cell lines may be most effectively transfected with conditions specific to that cell. In order to ensure that transfection studies with different cell lines were performed with conditions most appropriate to each cell line, a multiwell-based transfection approach was established to test variations in the amounts of liposomal reagent and of DNA.

The plasmid used for these studies consists of the CMV promoter driving the selectable marker gene blasticidin-S-deaminase. The reagent used to transfect DNA into the cells was Lipofectamine 2000. The plasmid, Lipofectamine, and blasticidin were obtained from Invitrogen.

Each cell line to be examined was trypsinized and plated in a 48 well plate in DME/F12 with a cell-appropriate serum amount. The cells were transfected when confluent by replacing growth medium with F12 medium with no protein and then incubating the cells with varying amounts of DNA, between 0 and 1.0 μg/cm , and a set concentration of lipofectamine. For another set of wells the DNA concentration was fixed and lipofectamine was varied between 0.1 and 2 μl/cm². With most cell lines lipofectamine at 2 μl/cm² (or 2 μl per ml or well, each well=1 cm² and 1 ml medium/well) exhibited toxicity, particularly when complexed with DNA. After overnight culture in transfection conditions the medium was replaced with culture medium. Selection with the cell-appropriate concentration of blasticidin was started within 1-2 days of transfection.

The viable cell mass of all wells was measured at intervals for two weeks to quantify growth and survival of cells with blasticidin selection. Dose response curves were plotted to assess optimal concentrations of DNA and lipofectamine to be used in future studies. The viable mass of resistant cells was used as an indication of conferred resistance. It should be noted that this approach provides for conditions that favor stable expression of transgenes. Transient expression does not as readily provide for cell growth with two weeks of selection.

B. Results

The cell line CRL 5803 exhibited a wide range of conditions that resulted in stable expression of blasticidin resistance conferred by the blasticidin deaminase gene (BSD). With 0.4 μg DNA/cm², 0.2 μl/cm² lipofectamine conferred good expression, increasing lipofectamine resulted in essentially maximal expression with 0.4 μl/cm² and no significantly better expression as lipofectamine was increased to 2 μl/cm². When lipofectamine was held constant, increasing DNA from 0.4 μg/ml to 1.6 μg/ml resulted in 13% increase in expression. These studies indicated that 0.4 μg/cm² of DNA and 0.4 μl/cm² of lipofectamine provide reasonably optimal conditions of transfection for CRL 5803 cells.

ARPE-19 cells appeared to have a fairly narrow DNA optimum of 0.4 μg/ml, and high lipofectamine requirement. A combination of 0.4 μg DNA and 0.4 μg lipofectamine resulted in poor expression, while increased lipofectamine combined with 0.4 μg DNA improved expression, with similar expression obtained with 0.8 or 1.2 μl lipofectamine. Either halving or doubling DNA from 0.4 μg/ml gave much poorer BSD expression. The results indicated that 0.4 μg DNA/cm² combined with 1 μl of lipofectamine/cm² represents optimal conditions for transfection of ARPE-19 cells.

Results with CCL205 cells indicated that with these cells lipofectamine is more critical, and DNA is effective at several concentrations. Lipofectamine at 1.0 μl/cm² was appeared optimal, while similar survivals were seen with 0.1, 0.2, and 0.4 μg/cm² of DNA when LF was constant. Transfection of this line appeared optimally accomplished with 0.2 μg/cm² of DNA and 1.0 μl/cm² of LF.

The CRL1440 cells also exhibited a fairly narrow DNA optimum of 0.2 μg/cm², halving or doubling the DNA resulted in markedly reduced expression (<25% of expression at 0.2 μg/cm²) in the context of nearly optimal lipofectamine of 0.8 μl/cm². Lipofectamine of 0.4 μl/cm² with 0.2 μg/cm² DNA resulted in reduced expression (<20%) compared to 0.8 μl/cm², while increased lipofectamine to 1.2 μl/cm² increased expression ≈20%. Optimal expression with CRL1440 cells appears to result when transfected with DNA at 0.2 μg/cm² combined with 1.2 μl/cm² of lipofectamine.

These studies indicate that the cell line CRL 5803 was the most robust in terms of stable transfection, in that neither the DNA nor the lipofectamine requirement exhibited a narrow concentration range for expression. Transfection of the other three cell lines requires optimized, specific DNA and lipofectamine combinations.

### Example 7

### Transgene Expression-Selection of Cell-Optimized Promoters

An important step in engineering a cell is delivering the DNA in such a way that the cell will incorporate the DNA into the cellular genome. Once integrated into the cellular DNA, regulatory elements of the cell and of the DNA construct become important. High-level expression of transgenes is routinely achieved by selecting the CMV enhancer/promoter to control the expression of the gene of interest, because the CMV promoter is regarded as providing high level expression in many tissues {Baskar et al., J Virol 70:3207-14, 1996). However, it is also recognized that the cellular context influences the activity of promoters {Smith et al., J Virol 74:11254-61, 2000), and thus some promoters act cell-specifically. In order to successfully use an engineered cell for protein delivery, optimal transgene expression is a critical factor. Two-fold gains in expression may reduce a device or implant size proportionately, and maximize cost-efficiency of cell-production.

The following studies were designed to determine whether the cells- CRL 5803, CCL205, ARPE-19, and CRL1440, were characterized by large, promoter-dependent differences in expression of a common transgene, and whether CMV was the strongest promoter in the context of human cells. We are aware of few documented comparisons of expression among human cell lines with the CMV promoter, and other promoters.

Experimental Approach

Each cell line was transfected under conditions optimized for that specific cell line, and with each of three different constructs. Within 2-3 days of transfection the cultures were selected with blasticidin at the appropriate selection concentration identified for that cell line.

The three plasmid constructs tested differed in the promoter used to control the expression of the BSD gene (commercial plasmids from Invitrogen). At the end of selection (10-14 days) the surviving cells were passaged to establish a B1sd-resistant polyclone. Each Blsd-resistant polyclonal cell line was analyzed for the capacity to grow in Blsd at varying concentrations. The Blsd dose-response curve was plotted (growth vs dose) and the IC50 calculated for each promoter construct. The IC50 was taken to indicate the average BSD enzyme expression and activity level, and to indicate the relative activity of the regulatory elements in each cell line context.

Regulatory Element/Promoter Strength Results

The CRL 5803 cell line readily produced Blsd-resistant polyclones with each promoter construct, however, there was a wide range of resistance in the polyclones among the promoters. The CMV promoter provided good resistance with an IC50 of 21 μg/ml. The EF-1α promoter provided enough expression for resistant growth, but lower expression than CMV with an IC50 of 3 μg/ml. The UBq promoter was the most robust in this cell line, with expression sufficient to provide an IC50 of 35 μg/ml. The control cells transfected with a plasmid that expresses NPT, (for G418 resistance), had an IC50 of 1 μg-Blsd/ml. In summary, the best promoter for the CRL 5803 cell line is UBq, conferring markedly better Blsd-resistant growth than CMV, and Blsd-resistant growth at doses 10-times that provided by the EF-1 promoter construct.

The ARPE-19 cell line did not achieve the level of expression seen with CRL 5803 cells. The IC50 for Blsd-resistant growth was 12 μg/ml with the CMV promoter driving BSD-gene expression, 4.5 μg/ml with EF-1a promoter, and 3 μg/ml for UBq promoter. Thus, with this cell line the CMV promoter provided the best expression, with EF-1α and UBq providing similar expression, but both much lower than CMV. The resistance conferred by the CMV promoter in ARPE-19 cells was lower than that produced by the CMV promoter in CRL 5803 cells. The best expression in ARPE-19 cells was about one third of the best achieved in CRL 5803 cells.

The CRL1440 cell line results indicated that the CMV promoter was minimally effective. Cells transfected with the CMV promoter construct had to be tested with a low concentration dose-response, with Blsd concentrations from 0.5 to 4 μg/ml in order to determine the IC50. The IC50 with CMV was 0.6 μg/ml, and culture for almost 3 weeks did not yield detectable growth of a resistant population with doses ≧3 μg/ml. In contrast, both the UBq and EF-1 promoter constructs provided high level expression, requiring higher Blsd doses (≧20 μg/ml) to determine the IC50 for the transfected cell lines. The IC50 for EF-1 promoter polyclones was 5 μg/ml, and the IC50 for UBq promoter polyclones was 21 μg/ml. Therefore, as with CRL 5803 cells, UBq was the promoter construct providing the best expression for BSD-resistance in CRL1440 cells. Similarly, the best expression in CRL1440 cells exceeded the best achieved with ARPE-19 cells. Although the best expression of BSD-resistance with CRL 5803 cells was greater than that with CRL1440 cells.

The cell line CCL205 had the lowest BSD expression of the four cell lines compared, with very little difference in resistance among promoter constructs. The growth-based IC50 was 0.6 μg/ml for all three promoter constructs. There was an indication that a small number of cells had higher level expression with the CMV promoter in that the IC90 for EF-1 and UBq promoter constructs was 0.8 μg/ml, while it was >3 μg/ml with the CMV promoter. This represents a small population of cells resistant to killing with doses up to 3 μg/ml with CMV-promoter polyclones.

These studies provide indications of effective expression and activity of an intracellular protein. The results indicate that the CRL 5803 cells more efficiently express the selectable marker Blsd and confer resistance more effectively than do other cells considered for CNS implant, including the reference ARPE-19 cells. Only with the EF-1 alpha promoter is the shift in resistance to Blsd similar between CRL 5803 and ARPE-19 cells. However, this is because the EF-1 promoter plasmid is less effective in the CRL 5803 cells than other promoter constructs.

The best promoters for CRL 5803 cells—UBq and CMV—outperform the best promoter for ARPE-19 cells—CMV—by 2-3 fold. The best promoter for CRL 5803 confers a 30-fold shift in the inhibitory concentration of Blsd with polyclones, the best promoter with ARPE-19 cells provides an 8-fold shift, implying that with these promoters, CRL 5803 cells express blasticidin deaminase from the BSD gene more effectively than do ARPE-19 cells. If BSD is indicative of other potential transgenic proteins, the CRL 5803 cells will be expected to provide better protein expression than ARPE-19 or the other cell lines examined. In addition, these results indicate that UBq can be used effectively with at least three of these four human cell lines.

### Example 8

### Transgene Expression—Expression of Secreted Proteins

The capacity to engineer “a cell” for efficient, stable and effective production of a therapeutic protein requires that “the cell” has a facility for 1) integrating a cDNA, 2) utilizing the promoter and other introduced regulatory sequences to provide sufficient RNA transcription, 3) sufficient mRNA stability and translation for synthesis of the transgenic protein, 4) abundance of subcelluar machinery (ER, Golgi, vesicles, proteolytic enzymes) for folding, sorting and processing of the transgenic protein, 5) effective means of secreting/releasing the active protein.

The following studies were conducted to directly compare the capability of the cell lines to express transgenic proteins that are secreted. The promoter expression studies indicated the ability to express a transgenic protein that is predominantly intracellular, in addition, with those studies the expression was determined with an indirect measure-conferred resistance to cytotoxicity. In this series of experiments, various assays were used to directly assess secreted protein expression, and release.

A. Assay for Intracellular Transgenic Protein-Experimental Procedure

The first approach involved the use of expression plasmids for VIP, FGF-7, and BDNF. Each of these genes were cloned (Mologen, Fuencarral, ES) from a substantia nigra cDNA library and delivered to cells in the same plasmid background. The standard plasmid used was comprised of the CMV promoter, a chimeric intron (chimera of the 5′-donor splice site from the first intron of the human b-globin gene and the branch and the 3′-acceptor splice site from an intron preceding an immunoglobin gene heavy chain variable region), sequences for a carboxy-terminal poly-histidine and finally, SV40 polyA sequence. The selection was provided by an SV40-NPT selection cassette in the same plasmid. The resulting expressed peptide has a poly-histidine tag on the carboxy-terminus of the protein, providing for capture of the transgenic protein with affinity matrices.

The cells were transfected with the respective plasmid DNA and then selected with G418. Individual growing colonies were picked after about 3 weeks and plated into wells of chamber slides (Falcon Labware). Unless there were fewer resistant colonies associated with the transfection, at least 48 colonies were picked. Once the cells settled and attached (usually 2-4 days) the chambers were removed and the cells were fixed with 3% paraformaldehyde for 0.5-1 hour (4° C.). The slides were then dehydrated with a 50%-70%-50% series of alcohol, rinsed with PBS, and blocked with a protein solution in Tris-buffer to prepare for reaction with primary antibodies. Once the reaction with primary antibodies was complete, the slides were washed with Tris-saline-Triton X-100 buffer (pH 8.0), and the primary antibody binding was demonstrated using an alkaline phosphatase-labled second antibody. Antibodies for BDNF, FGF-7, and VIP were obtained from Santa Cruz Biotechnology (Santa Cruz, Calif.). The antibody dilutions were determined using non-engineered cells, and polyclones of transfected cells. Secondary antibodies were obtained from Santa Cruz, or Sigma.

The number of protein-expressing colonies was determined for each transgene, and the percentage of expressing colonies calculated for each transgene with each cell line.

B. Results For Intracellular Transgenic Protein

Transfection of CRL1440 or CRL 5803 cells provided hundreds of colonies, while with both ARPE-19 and CCL-205 it was very difficult to obtain resistant colonies. Typically fewer than 20 colonies were obtained with ARPE-19 and CCL-205 cell lines, and typically with G418 concentrations at which not all cells were killed in control plates (plates transfected with a plasmid not conferring G418 resistance). Both the CRL1440 and the CRL 5803 cell lines expressed BDNF in >50% of the picked clones. With CCL205 and ARPE-19 cell lines it was difficult to identify clones that expressed above background levels found in non-transfected cells.

A high percentage of CCL5803 cells expressed detectable FGF-7 (44%) or VIP (88%), paralleling results with BDNF. CRL1440 cell colonies also expressed these two proteins. As with BDNF, no clones expressing above non-transfected cells could be identified with CCL205 or ARPE-19 cells.

The results of these studies demonstrated that the cell lines CRL 5803 and CRL1440 were much more robust than ARPE-19 or CCL205 cells in the ability to express an introduced transgenic peptide. This was observed, even though the CMV promoter used is the better promoter for ARPE-19, but is not the best promoter for expression in CRL 5803 or CRL1440 cells.

C. BDNF Expression in Polyclones

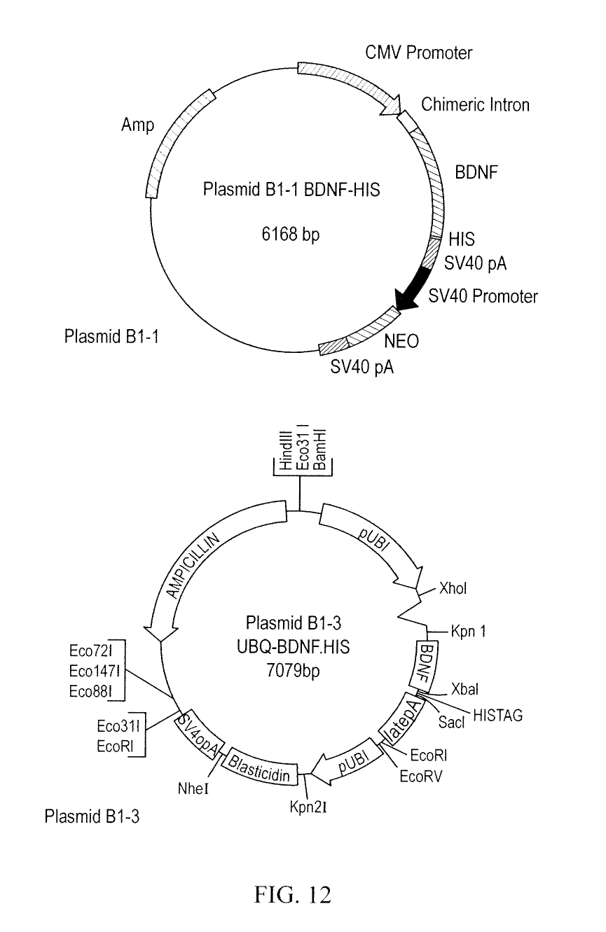

In a second approach the relative capability of each cell line to express a transgene was determined by transfecting a BDNF plasmid into each cell line, selecting for antibiotic resistance, and assaying for secreted protein. The plasmid construct (B1-1) set forth in FIG. 12 was comprised of a CMV promoter, a chimeric intron (chimera of the 5′-donor splice site from the first intron of the human b-globin gene and the branch and the 3′-acceptor splice site from an intron preceding an immunoglobin gene heavy chain variable region), the BDNF coding sequence (Access# NM_{—}001709) cloned from a human substantia nigra cDNA library, fused to a poly-Histidine tag, and with SV40 polyA signal. In the same plasmid, selection was provided by a SV40-Neo selection cassette.

Control plates were transfected as described above, except the plasmid used had no gene insert, and supplied the RSV-Zeo selection cassette.

Selection was with G418 (Invitrogen) at 500 mg/ml for CRL1440 and CRL 5803 cells, 120 mg/ml G418 for ARPE-19 cells and 15 mg/ml G418 for CCL205 cells. This selection was maintained at least 3 weeks, and then surviving cells were passaged and grown as polyclones.

Finally, as an index of both expression and secretion from the cells tranfected with BDNF plasmid, the cells stable expression of BDNF was assayed with these cells. Polyclones of the stable cells were grown in 10 cm dishes(two dishes for each cell line) and the supernatant medium was collected. The functional cell mass in the cell monolayers was determined with an assay that involved incubation with resazurin (40 μM) for 30 minutes, sample collection at 0, 15, and 30 minutes and then measurement of the resorufin produced. The cells were rinsed with PBS and removed with 1× trypsin-EDTA (0.05 ml/cm²). The pellets were washed, then suspended in lysis buffer [20 mM K2HPO4, 1 mM EDTA, 110 mM KCl, 0.5% triton X100, pH 8.0, with proteolytic inhibitors] and frozen at −80° C.

The conditioned medium collected from non-transfected cells and the polyclones transfected with BDNF-HIS were divided into two equal aliquots and concentrated for analysis in two different ways. In the first method, the medium sample was concentrated by methanol precipitation. The second preparation involved incubation of the sample with poly-histidine affinity beads. Methanol will precipitate all BDNF, the affinity beads capture transgene HIS-tagged BDNF, allowing for specific concentration of transgene product. In this way endogenously produced BDNF can be distinguished from BDNF that is a product of the introduced transgene.

MetOH precipitation: A portion of the 24 h conditioned medium was collected and mixed with methanol equivalent to 20% of the volume (final volume 120%). The samples were incubated 10 minutes on ice to complete precipitation Subsequently the samples were centrifuged for 5 min at 13000 rpm (16060×g). The methanol medium supernatant was removed and discarded. The pellet was allowed to stand 5 min at RT and then prepared for western analysis.

HIS-tag affinity extraction from cultured medium: EZ Red affinity beads (Sigma Aldrich) were used to capture poly-histidine tagged proteins present in the conditioned medium of the BDNF-HIS transfected polyclones. Medium was incubated at 4° C. with shaking for 24 h in the presence of affinity beads. The incubation mixture consisted of 4 volumes of medium, 10 volumes of equilibrated beads, and 10 volumes protease inhibitor (P8849, Sigma Aldrich) with 1 mM imidazole. At the end of 24 h, the beads were pelleted and washed twice with cold TBST (1.5M NaCl, 100 mM Tris-HCl pH 8.0, 5% Tween 20) and eluted in imidazole buffer (50 mM NaH2PO4, 250 mM imidazole, 300 mM NaCl) by shaking the bead and buffer mixture at 4° C. for 15 h. The beads were pelleted and the supernatant was collected. The supernatant was then mixed with 20% methanol to concentrate eluted proteins for analysis by western blot.

HIS-tag affinity extraction from cell pellet: Cell pellets were collected from two 10 cm dishes and suspended in 10 ml of buffer and frozen at −80° C. The cell pellet samples were frozen and thawed twice, and then cell debris was pelleted by centrifugation for 15 min at 1000×g. The resulting supernatants were collected and incubated with affinity beads and prepared for western analysis as described for the media samples.

The final precipitates of the concentrated medium samples were loaded onto a 4-12% precast gel (Invitrogen) for protein separation and western blotting.

D. BDNF Expression Results

All parental cells appeared to produce endogenous BDNF at low levels, even when cultured in 0.1% FBS. The ARPE-19 cells had the highest expression of endogenous BDNF, with higher molecular weight precursor forms also detectable.

The transfected, and selected polyclones of both CCL205 and ARPE-19 produced no detectable BDNF-HIS protein. Although BDNF could be detected in low amounts when medium (5 ml) was methanol precipitated, no BDNF was detectable after affinity capture. In contrast, affinity captured BDNF was readily demonstrated in medium samples from CRL1440 and CRL 5803 cells transfected with BDNF-HIS expression plasmid. The lack of detectable expression with ARPE-19 cells was not due to vastly different cell numbers, the viable cell activity assay with resazurin indicated that the functional volume of cells in the confluent cultures of the different cell lines were too similar to be distinguishable.

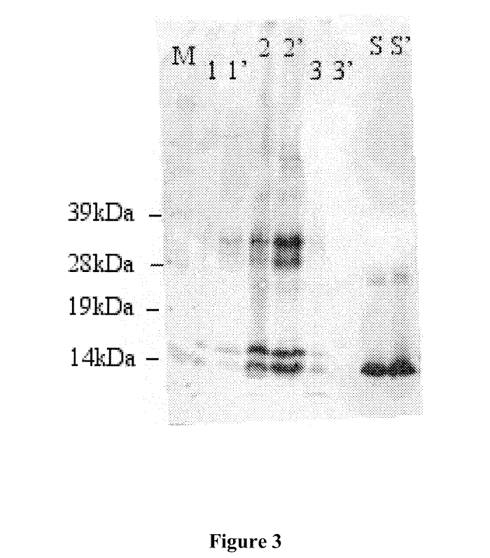

The western blot shown in FIG. 3 illustrates the detection of methanol precipitated BDNF and affinity-captured BDNF for CRL1440-, CRL 5803-, and ARPE-19-derived cells. The blot demonstrates that CRL 5803-derived cells secrete readily detectable immunoreactive BDNF (iBDNF), whether affinity-captured, or methanol-precipitated. Although produced in lower amounts, iBDNF was detectable in both samples from CRL1440-derived cells as well. As indicated by methanol-precipitable iBDNF in lane 3, but no BDNF affinity-captured-lane 3′, the BDNF produced by ARPE-19-derived cells is the product of an endogenous gene. This blot also illustrates that the CRL 5803-derived cells process the BDNF-HIS transgene, with two precursor species, 30 kDa range, and two processed species, 14-15 kDa. These bands are also present, though less intense, with ARPE-19 and CRL1440-derived cells. The higher molecular weight bands likely represent pro-BDNF and glycosylated pro-BDNF, while the 14-15 kDa forms likely represent mature BDNF, similar to processing observed in neurons {Mowla et al., J Biol Chem 276:12660-66, 2001).

The image in FIG. 3 was used to quantify (ImageJ software; http://rsb.info.nih.gov/ij/) the density of the various immunoreactive BDNF species, and these data were used to compare the expression of BDNF among these cell lines. The affinity-captured iBDNF data was used to compare BDNF-expression in CRL1440- and CRL 5803-derived lines. The polyclone of CRL 5803 transfected with BDNF-HIS secreted 5.7 times as much transgene iBDNF as did the CRL1440 polyclone. The methanol-precipitated iBDNF data was used to compare the endogenous production by ARPE-19 cells and transgene BDNF of CRL 5803 polyclonal cells. The CRL 5803 polyclonal cells secrete more than 10 times as much iBDNF transgene product as the ARPE-19 cells secrete of endogenously produced iBDNF.

Western blot analysis of cell pellets provided parallel data (not shown). The CRL 5803-derived polyclones also contained readily detected transgene iBDNF intracellularly, and more than any of the other cell lines. ARPE-19 cells contained no detectable transgene iBDNF.

The results of this study provides another example that CRL 5803 cells provide a better expression system than other candidate cells examined, or the ARPE-19 cell line that has been used to deliver therapeutically effective protein by others. This Example not only demonstrates that the CRL 5803 cells outproduce other cell lines in terms of transgene peptide production, but also in terms of transgene peptide secretion into the medium, surpassing other cell types 6-10 fold. This study also indicates that CRL 5803 cells also have the capacity to proteolytically process pro-BDNF to mature BDNF.

### Example 9

### Role of Promoter Elements in Production of Resistant Clones

In many of the studies, the lack of expression with ARPE-19 cells and CCL205 cells was associated with fewer colonies that could be picked, and poor growth of isolated clones. This indicated that the expression of the selectable marker, NPT, could be inadequate. In an effort to address this issue we used plasmids we had constructed that used either of three viral promoter/enhancers: CMV, SV40, or RSV. These viral regulatory elements were used to direct the expression of the selectable marker for Zeocin, a bleomycin binding protein (BBP) that inactivates the drug by binding, rather than enzymatic degradation. The resistant colonies produced by these transfections were grown as polyclones for each different promoter. These polyclonal cells were plated and grown at varying Zeocin concentrations of 100, 300, 600, 900, and 1200 μg/ml, (or up to 5000 μg/ml if higher concentrations were necessary) and growth was measured to determine IC50s as an index of resistance marker expression.

CRL 5803—Zeocin-resistant colonies and thus polyclones were readily established with the CRL 5803 cell line. All promoters provided about a 5-fold increase in the growth IC50. The IC50s were: 2700 μg/ml for CMV, 2400 for RSV, and 2800 for SV40 promoter plasmid. The dose range was extended to 5000 μg/ml for the CMV promoter polyclone, and it was found that cytotoxicity was evident only with doses >3000 μg/ml. The results of these transfection studies indicate the capacity of the CRL 5803 cells to use these viral promoters. These viral promoters in CRL 5803 cells were fairly equivalent, providing high dose resistance even with a selectable marker that does not provide for enzymatic degradation of selection drug.

ARPE-19—No clones could be established with ARPE-19 transfected either with SV40 or RSV promoter-Zeocin resistance (BBP) plasmids. However, with the use of the CMV promoter resistant cells were obtained. The colonies were grown in 250 μg/ml Zeocin, and pooled to constitute a polyclonal population of cells stably expressing BBP/Zeo-resistance. The IC50 for growth was 399 μg/ml, this represents about an 8-fold shift in resistance compared to ARPE-19 cells with no Zeocin-resistance gene. These results suggest that the poor yield of resistant colonies in G418 with SV40-NPT cassette, is due to inadequate expression of NPT by the SV40 promoter in these plasmids. As a result, the likelihood of finding a clone with high-level expression for the gene of interest is dramatically reduced.

CRL1440—Zeocin-resistant clones of CRL1440 cells were produced with SV40 promoter and CMV promoter plasmids, but none were obtained with RSV promoter plasmids. Polyclones were expanded in 300 μg/ml Zeocin for SV40 and CMV promoter plasmids, and then plated to determine IC50s for growth at varied Zeocin doses. The calculated IC50s were 959 μg/ml for SV40 promoter plasmid and 2395 μg/ml for CMV plasmids. This result provides an example of another human cell line that does not effectively use RSV promoter. In addition, the CRL1440 cells effectively use the SV40-Zeocin cassette to confer marked resistance, even though the expression was considerably less than that provided by the CMV promoter.

CCL205—Resistant CCL205 cells were not established with the RSV promoter, and although resistant cells were obtained with the SV40 promoter, the cells grew too poorly to determine an IC50. The growth of CCL205 cells was slow enough that for IC50 calculations the final viable mass was used rather than growth. The CMV promoter provided resistant cells, although in the polyclone there was not a marked shift in resistance to Zeocin, with an IC50 of 27 μg/ml.

The results of these studies with viral promoters suggest that in human cell lines some viral promoters are minimally effective. This is particularly important with respect to the SV40 promoter. The SV40 promoter is often used in plasmids for selection cassette expression to provide resistance to selectable markers, and thus aid in the isolation of stable clones that express the transfected plasmid. In many of the studies of the ARPE-19 cell line in these examples, the selection resistance depended on SV40 promoter activity. A strong inference of these results is that the use of ARPE-19 cells for engineered protein expression should preferably not use the SV40 promoter to control expression of selectable markers or transgene peptides.

Finally, these studies also indicate that with another transgene, the CMV promoter does not provide expression in ARPE-19 cells to match that of CRL 5803 cells (or even CRL1440 cells). These results emphasize the robustness of the CRL 5803 cell line, in that all promoters examined were effectively used with this cell line. Therefore, promoter studies with a cell line like CRL 5803 are directed to finding an optimal promoter, while with a cell line such as ARPE-19, studies should be directed to eliminate promoters that are ineffectual for use in transgenic engineering.

### Example 10

### Expression of Secreted Marker Enzyme

The purpose of the studies in this section was to test/screen cells for the capacity to effectively synthesize and release an indicator protein- secreted alkaline phosphatase (SEAP). This gene was chosen because the product is an enzyme that readily allows for direct measurement of expression of both intracellular and extracellular product, in addition it is an enzyme that is well recognized for its capacity to be secreted. Use of this gene and enzyme provides a model whereby both the facility of “the cell” to be engineered and the secretory capabilities of “the cell” can be measured readily. A fluorescent-based assay also provides for highly sensitive detection of the secreted and intracellular product.

The data from all previous expression studies indicate that ARPE-19 and CCL-205 cells do not express proteins as well as CRL1440 or CRL 5803 cells. This was true for selectable marker enzymes, and also for secreted proteins such as BDNF, VIP, and FGF-7. The ARPE-19 cells are known to express endogenous BDNF {Kanuga et al., Invest Ophthalmol Vis Sci 43:546-55), therefore the lack or low level of transgene BDNF expression does not appear to be an inherent translational problem in these cells. The use of SEAP plasmids provide an alternate means to compare the expression and release of secreted transgene protein with all four cell lines.

It should be noted the measurement of secretion in these studies indicate that the tested cell line has the facility for 1) integrating a cDNA, 2) utilizing the promoter and other introduced regulatory sequences to provide sufficient RNA transcription, 3) sufficient mRNA stability and translation for synthesis of the transgenic protein, 4) abundance of subcelluar machinery (ER, Golgi, vesicles, proteolytic enzymes) for folding, sorting and processing of the transgenic protein, and 5) effective means of secreting/releasing the active protein.

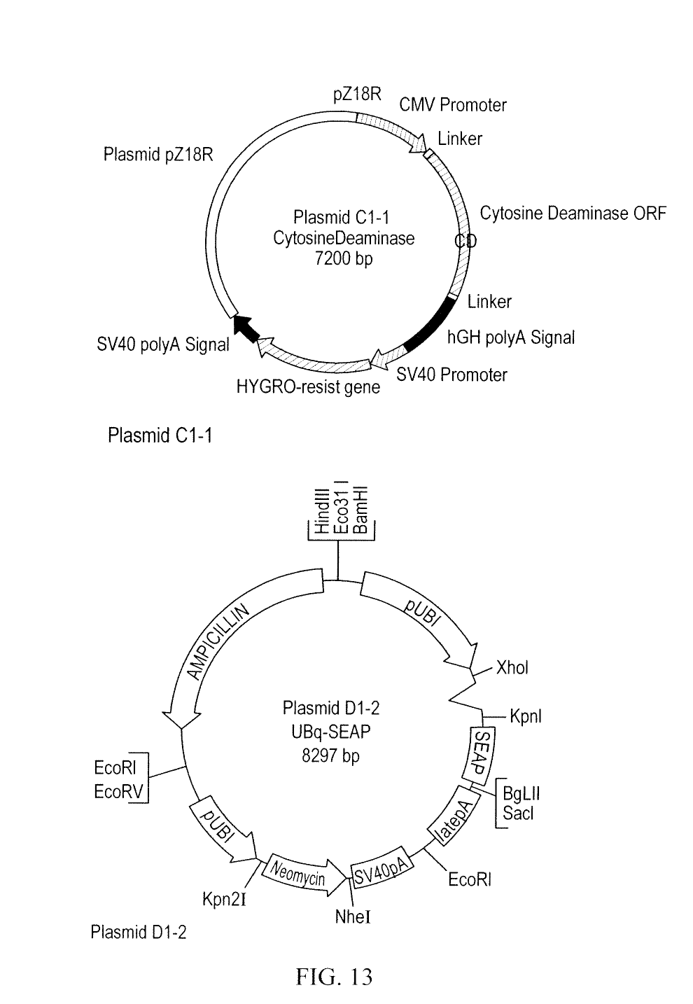

The plasmid (UBq/AlkPhos; Plasmid # D1-2, set forth in FIG. 13) used for transfection in these studies utilizes the ubiquitin promoter for expression of both the gene of interest-SEAP, and for the selectable marker-neomycin phosphotransferase (NPT). The use of the same promoter for both the selectable marker and the gene of interest helps to avoid the difficulties encountered with ARPE-19 cells for plasmids that utilize SV40 or RSV promoters for selectable marker expression. The drug G418 was used at concentrations of 250-500 μg/ml (active G418 concentration) to select for cells that stably expressed the transfected plasmid DNA (except for the CCL205 cell line where the selection was lower due to high sensitivity to G418).

SEAP assay—The serum used to supplement the culture medium contains appreciable alkaline phosphatase activity, even when used at low levels of 1% supplementation. To insure measurement of SEAP produced by the cells, samples for assay were collected from cell cultures that had been provided medium with either 0.05-0.1% FBS and background corrected, or with no serum supplementation. The medium was assayed for SEAP activity using the fluorescent substrate umbelliferyl phosphate. The substrate was dissolved in assay buffer-100 mM Tris buffer at pH 9 with 5 mM magnesium and 0.1 M NaCl. The substrate and sample for assay were mixed in a well of a 96 well plate and the fluorescent umbelliferone released by the activity of SEAP was measured kinetically in a SpectraMax M2 plate reader (Molecular Devices, Sunnyvale Calif.). The software (SoftMax Pro, Molecular Devices) determines the rate of reaction and calculates the Vmax for each well. The Vmax for each cell line was used to indicate the expression of SEAP with each cell line assayed. In addition, in most studies the resazurin assay was used to determine the functional cell mass within the culture.

The cells that were used for comparison included five human cell lines: CCL205, CRL1440, CRL 5803, the reference cell line ARPE-19 and a rapidly growing cell line-A2058. The cells, monolayers in 6 well plates, were transfected with plasmid using conditions that had been identified as optimal for each cell type, and drug selection was started 1-2 days after transfection. Non-transfected controls were included to indicate when all cells not expressing the resistance cassette would be killed. With each cell line, except the CCL205 cells, the resistant colonies were allowed to grow to a near confluent monolayer and a sample of medium was collected to assay for released SEAP. The CCL205 cells had too few resistant colonies to readily grow to confluence and were passaged to expand the cells for further study. The monolayers in 6 well plates were passaged as polyclones to expand the new cell lines, with all lines SEAP activity was detectable before passage.

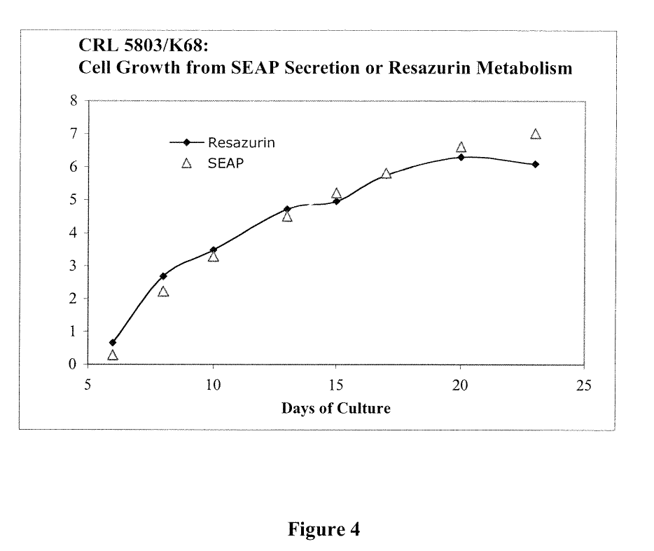

The four rapidly expanding cell lines, CRL1440, CRL 5803, ARPE-19 and A2058 were used to compare SEAP release with the growth rate of the cells during log phase growth. The cell lines produced by transfecting the UBq/AlkPhos plasmid were lines K65.p (derived from ARPE), K67.p (from CRL1440), K68.p (from CRL 5803), and K69.p (from A2058). The cells were plated in multi-well plates at ≧1:4 split ratios and the growth of each well was determined with the resazurin assay at 1-3 day intervals. Medium samples were collected for the same intervals and assayed for SEAP activity. The CRL 5803/K68 line and the other cell lines, with the exception of ARPE-19 cells, produced increases in medium SEAP in proportion to the increase in cell number (FIG. 4). There was a significant correlation between SEAP activity and resazurin activity with r2=0.72, 0.88, 0.83, for CRL1440, CRL 5803, and A2058, respectively. The SEAP activity in the ARPE-19/K65.p cultures remained close to the limits of quantification, no matter the cell density. These results indicated that the release of SEAP, with three cell lines that made measurable quantities, increased as cells grew and did not decline with cultures at plateau density. These studies also indicated which cell line made the most SEAP on a cell mass basis: CRL 5803≧CRL1440>A2058>>ARPE-19.

It should be noted that the CRL 5803 cells transfected with SEAP represent cells with a reporter gene that would allow these cells to be used to measure SEAP production and release and by this means evaluate conditions that influence Ubq promoter activity, or that influence SEAP synthesis and release, either in vitro or in vivo. For example, the cells could be used to effect of the effect of calcium channel blockade (verapamil or other agents) on SEAP release.

### Example 11

### Cloning and Expression of a Suicide Transgene

These studies had three purposes. The first purpose was to determine whether the bacterial gene, cytosine deaminase, could be expressed using the native gene sequence without mutating the initial amino acid of the coding sequence to a methionine to provide for expression in mammalian cells. It has been suggested that the upstream ATGs in the native CD gene would result in poor gene expression in eukaryotic cells, and therefore the coding sequence for the initial amino acid must be mutated for mammalian expression (Mullen et al., Proc Natl Acad Sci USA 89:33-7, 1992; U.S. Pat. Nos. 5,358,866, and 5,624,830). However, we are unaware of any other than theoretical examination of this possibility. A second purpose was to use the CMV promoter in the absence of the artificial intron that may have been problematic for SEAP expression. The third, and most relevant purpose was to examine the feasibility of expressing a suicide gene to provide for targeted killing of engineered cells.

A. Cloning the CD Gene

Genomic DNA was isolated (Dneasy Tissue Kit; Qiagen, Valencia, Calif.) from E. coli (XLII Blue, Stratagene, La Jolla, Calif.) grown in Loria Broth medium for ≈16 h.

Two oligonucleotides were synthesized (Life Technologies Gibco BRL, Gaithersburg, Md.) to amplify the full-length CD gene. The 5′ primer CD1, is 24 nucleotides long, and represents base-33 to base-10 of the published sequence of the CD gene (Austin et al., Mol Pharmacol 43:380-7, 1993): -33 CGA CAG CAG CAA TGA CGC ATG TGG-10 (SEQ ID NO: 1). The underlined ATG is fifteen codons upstream of and in frame with the bacterial start codon GTG at position +1. The 3′ end primer, CD2, is 25 oligonucleotides long representing base 1322 to base 1297 of the published sequence: 1322-CGC ATC CGG CAT AAA CTA AGC TCG C-1297 (SEQ ID NO: 2). This primer is 14 nucleotides down stream of the stop codon.

The amplified 1356 base pair fragment, representing the CD gene, including 33 bases 5′ UTR and 38 bases of 3′ UTR, was separated by electrophoresis, recovered from the agarose gel and blunt end cloned into a cloning vector (pNoTA/T7; 5 Prime-3P Prime, Boulder Colo.) to produce plasmid CF626. The gene was removed from CF626 and transferred to the plasmid pCB7 (Clark et al. Diabetes, 46:958-67, 1997) with the restriction endonucleases Kpn1 and Hind3 to give CG836. Plasmid CG836 was sequenced and the sequence indicated that cloning resulted in a SalI site involving nucleotides-33 to -30 of the wild-type CD sequence. The gene was removed from CG836 with the restriction endonucleases SalI and XbaI and cloned in SalI, XbaI sites of plasmid pCB7 to produce the expression plasmid CK521. This plasmid provides the CMV promoter to drive expression in eukaryotic cells. The insert in this plasmid was sequenced. Alignment of the nucleotide sequence of CK521 with the published bacterial cytosine deaminase sequence (Austin et al., Mol Pharmacol 43:380-7, 1993, the disclosure of which are incorporated herein by reference) indicated that the two are identical from −33 to +1356 of the wild-type CD sequence (SEQ ID NO: 3).

Initial studies of 5-FC sensitivity indicated that cells transfected with CK521 were not sufficiently growth inhibited when cultured in 5-FC (see subsequent Examples for characterization). Two additional expression plasmids were created in an effort to provide an expression plasmid with high enough expression to result in killing of cells when cultured in 5-FC.

B. Expression Plasmid Modifications

Plasmid CW609: This expression vector was made in an effort to increase amino-extended CD expression. This was accomplished by reducing the amount of wild-type sequence 5′ of the most distal ATG of the wild-type CD sequence in the CK521 plasmid. In this process, the stop codon upstream of the ATG was mutated (TGA to GGA).

The oligonucleotide CD6 was synthesized this includes bases-33 to -4 of the wild-type CD gene: ATG CGT CGA CAG CAG CAA GGA CGC ATG TGG AGC CTA (SEQ ID NO: 4). It provides a SalI site at the 5′ end and changes the TGA stop codon upstream of the ATG to GGA. The oligonucleotides CD6 and CD4 (CGC CGT TCC CAG CGG ATA CCA CGG, SEQ ID NO: 5)) were used to amplify a portion of the CD gene. The resulting 1 kb PCR fragment was cloned into pBlue Script KS2 to create plasmid CU519. Plasmid CU519 was digested with the restriction enzymes SalI and BssH2 and this fragment of the CD gene was cloned into the same sites of CK521 creating plasmid CV621. Polylinker of plasmid CV621 was deleted by digesting CV621 with Bgl2 and Hind3 restriction endonuclease and filling the ends using calf intestinal polymerase. The plasmid was then ligated back on itself creating plasmid CW609. This plasmid, CW609, has 42 nucleotides upstream of the ATG, 14 nucleotides of the wild type sequence, the remainder is polylinker. In addition, a mutation was introduced at −21 to provide GGA instead of the stop codon TGA upstream of the ATG.

Plasmid CX014: This expression vector was made in an effort to increase amino-extended CD expression. The intent was to optimize the sequence between the polymerase binding site and the mammalian start codon (ATG) in the 5′ sequence of the bacterial start in the CD gene. This was accomplished by deletion of wild type sequence upstream of the ATG and most of the polylinker sequence in the expression vector.

Two oligos were designed and several intermediate cloning and expression vectors were made for this purpose. Oligo CD8 is a sense oligo which includes between bases -15 to -4 of CD gene (AAT GAC GTC GAC ATG TGG AGG CTA, SEQ ID NO: 6) and provides a SalI site prior to the the −15 ATG. Oligo CD4 is a 3′ oligo that extends between nucleotide 1084-1107. CD8 and CD4 oligos were used to amplify 1 kB of CDase gene from the genomic DNA of XL2 blue cells. A 1 kB fragment, representing part of cytosine deaminase gene, was separated by electrophoresis, isolated from the agarose gel and blunt end cloned into a cloning vector (pNoTA/T7) this produced plasmid CU613. A portion of the CD gene, 0.74 kB, was then excised from plasmid CU613 using restriction endonucleases SalI and BssH2. The 740 base fragment replaced the Sal1-BssH2 fragment of plasmid CK521 creating plasmid CU916. Plasmid CU916 has 14 nucleotides less than plasmid CK521 upstream of the ATG. CU916 has polylinker sequence between the Bg12 and the SalI sites.

The polylinker sequence upstream of the Sal1 site in plasmid CK521 was deleted by digesting with Bgl2 and Hind3 restriction endonuclease and the ends were filled by calf intestine polymerase. The plasmid was then ligated back on itself creating plasmid CV745.

The final plasmid with deleted 5′ wild type and polylinker sequences was made by digesting plasmid CV745 with Spe1-Sa1I and this fragment inserted in place of the Spe1-Sal1 fragment of Plasmid CU916, creating plasmid CX014 (SEQ ID NO: 7). Plasmid CX014 has the bacterial CD gene in bases 943 to 2295 of this plasmid, with the ATG at -15 to -13 of the bacterial gene providing the equivalent of a mammalian start codon.

C. CD Antiserum and CD Protein Expression in Cell Lines

Different cell lines exhibit various sensitivities to 5-fluorouracil. Overexpression of cytosine deaminase may not provide similar potencies of killing with different cell lines. Differing potencies could be related to factors limiting sensitivity of the cells to 5-fluorouracil—inability to adequately phosphorylate 5-fluorouracil, high levels of thymidylate synthase expression, high levels of dipyrimidine dehyrogenase, as well as lower expression or even non-expression of the cytosine deaminase transgenic protein. In order to discriminate among some of these possibilities it is useful to be able to assay for expression of the transgenic cytosine deaminase protein, and thus ensure that cytotoxicity or lack thereof, is due to expresssion (or lack thereof), transcriptionally and/or translationally, of the cytosine deaminase transgene. Polyclonal antibodies to cytosine deaminase were developed to provide an indication of protein expression for both wild-type and amino acid-extended cytosine deaminase molecules.

D. Peptide Sequences and Antibody Production

The peptide sequence of cytosine deaminase was predicted from the published nucleotide sequence for cytosine deaminase. The translated sequence, hydrophobicity, and antigenicity plots were generated using commercial molecular biology software (MacVector 4.5). Two peptide sequences were selected, (Pep4 and Pep5) and synthesized (Biosynthesis, Lewisville, Tex.). The Pep4 peptide extends from amino acid 56 to amino acid 85 (PPFVEPHIHLDTTQTAGQPNWNQSGTLFEG, SEQ ID NO: 8), and the Pep5 peptide extends from amino acid 278 to amino acid 302 (LVNIHLQGRFDTYPKRRGITRVKEM, SEQ ID NO: 9) of the wild-type CD protein. Antibodies to a mixture of the two peptides were raised in rabbits.

E. Western Blot Analysis

Cross-reactivity of antibody with CD of bacteria and CD cell lines.

Plasmid BX600 was from the ATCC (#40999; pCD2, Genbank X63656). This plasmid was used to express a mutated CD cDNA in rodent cells and compare expression with that of the non-mutated, however 5 amino acid-extended, CD protein of the present invention. The plasmid BX600 uses neomycin phosophotransferase gene (NPT) as the selectable marker. The plasmid was electroporated into the RIN1046-38 cell line, and selected with the NPT drug, G418, creating the KB231 cell line. Alternately, BX600 was electroporated into 131/624 cells, selected with G418 to create KB234 cell lines. These cell lines were used to compare the expression of the expressed CD protein with that of the protein of the present plasmid.

Cell pellets were suspended in 50-100 μl of PBS buffer with 1 mM EDTA and proteolytic inhibitors, and sonicated at 30% power with 5 short bursts. Cell lysate protein was loaded at 1-25 μg/well and electrophoretically separated in Tris-glycine 4-20% polyacrylamide mini-gels (Invitrogen). The proteins were transferred to PVDF membranes, the membranes were blocked with 4% powdered milk+0.4% BSA in Tris-borate saline+EDTA (TBST-milk) for 0.5 h, then incubated with 1:1000 to 1:4000 dilution of polyclonal CD antibody for 4 h. The membrane was washed in TBST-milk after incubation with anti-CD, and after the wash incubated 1 h in alkaline phosphatase-coupled goat anti-rabbit IgG. Membrane was washed then color was developed in Tris-saline-Mg buffer (pH 9.5) with nitroblue tetrazolium (NBT; 3.3 mg/ml mg/ml) and 5-Bromo-4-chloro-3-indolyl phosphate (BCIP; 1.6 mg/ml).

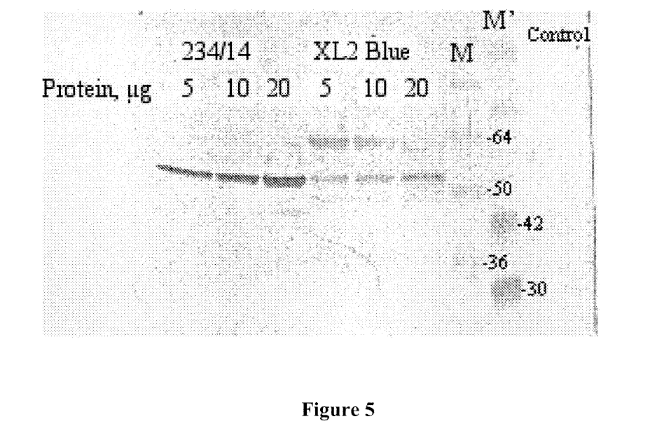

The size of cytosine deaminase, based on predicted amino acid sequence, is ≈47 kD. Trial bleeds tested on E-Coli lysate and on cell lines transfected with cytosine deaminase transgene exhibited cross reactivity with a protein band of approximately ≈53 kD. In E. coli an ≈53 kD immunoreactive band was apparent with 1:1000 dilutions of cytosine deaminase antiserum. There was some additional reactivity with an ≈59 kD material in E. coli lysates, but only the ≈53 kD material increased reactivity proportionately with increased total protein between 5 and 20 μg/lane (FIG. 5).

The cross reaction with the ≈53 kD material was specific at antibody dilutions of 1:1000 with KB234 cells transfected with cytosine deaminase transgene, and proportional to total protein loading with 5-20 μg cellular protein (FIG. 5). KB302 cells weakly expressed a protein that migrated at 53-54 kD, detectable with antibody dilutions of 1:1000 and 10 μg of protein, (almost undetectable with 1:4000 dilutions). Nontransfected RIN1046-38 cells had no reactive material under these conditions. Finally, specificity of the antibody was demonstrated by loss of the crossreactivity of 53 kD material when the antibody was blocked by pre-incubation of the antiserum with excess of Pep4 and Pep5.

F. Effect of Different CD Plasmids in Rodent Cell Lines

Plasmid CK521 was electroporated into RIN1046-38 cell line to create KB302 cell lines. Cells were selected in hygromycin, and surviving colonies picked. Plasmid CW609 was electroporated into KB 131/624 cell line, and cells were selected in hygromycin to creating the KB570 series of cell lines. Plasmid CX014 was electroporated into KB 131/624 cell line, and selected with hygromycin to create KB571 series of cell lines.

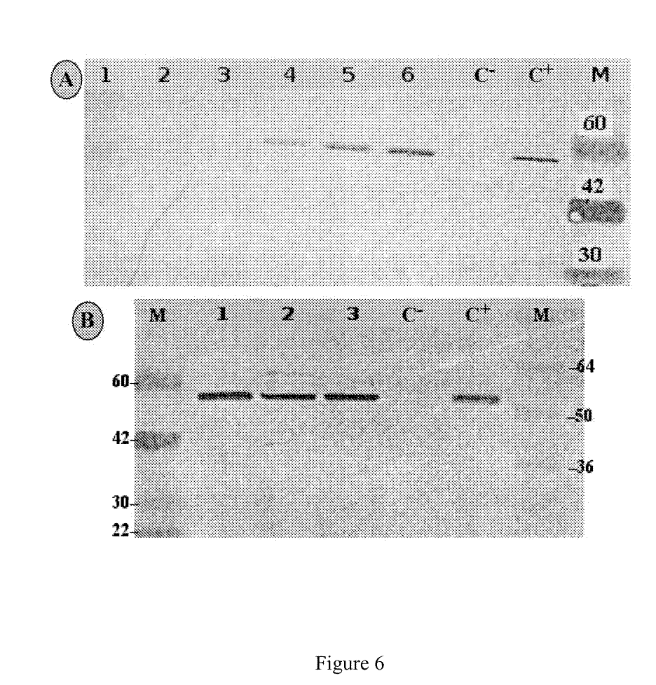

The cell lines from KB302, KB570 and KB571 were expanded and cells harvested for western analysis. Western analysis indicated that the level of expression in polyclonal KB570 cells was better than the level of expression of KB302, lanes loaded with 5 μg of KB570 cells gave more intense immunoreactivity than 15 μg of KB302 cells (not detectable in the scanned image; FIG. 6A). KB571 polyclones exhibited CD expression that was higher than that of KB302 or KB570 cells (FIGS. 6B). This is indicated by the fact that immunoreactivity was saturated when 5 μg of KB571 protein was loaded per lane, with no further increase in signal with 10 or 15 μg in a well. In contrast, the most intense immunoreactivity with KB570 cells was at 15 μg of protein. These results indicated that the modifications to reduce upstream sequence in the expression plasmids provided increased levels of the 5′-extended CD gene sequence.

Furthermore, in analyzing individual clones other differences were noted between KB570 and KB571 cell lines. Analysis of 96 KB570 clones indicated that 30% were positive for CD and 34% of these positives were high expressers of CD protein. Analysis of 96 KB571 clones indicated that 70% of the clones were positive, and that 76% of the positives were high expressers. These results indicated that not only does the plasmid CX014 provide for the highest expression of CD protein per cell, but also the highest percentage of cells expressing detectable CD protein in this rodent cell line.

G. Wild-Type Size CD Gene and 5′-Extended CD Gene: Comparison of RNA and Protein Size

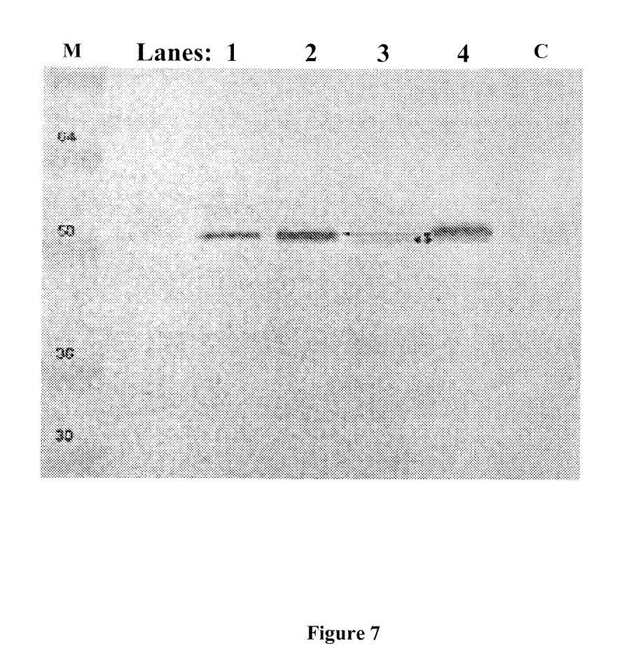

Cell lysate from KB234/14 cells, representing the wild-type size of CD protein, was analyzed by western blotting in comparison to cell lysate from KB571/10 cells. Each lane of a 10% polyacrylamide gel was loaded with equal amounts of lysate protein, an additional lane was loaded with a equal mixture of protein from both cell lines. FIG. 7 illustrates that the small size difference between amino-extended CD from 571/10 cells and mutated, wild-type size CD from 234/14 cells. The 427 amino acid size CD protein (SEQ ID NO: 13) (lane 3) ran slightly faster than amino-extended CD (lane 1, 2), and could be clearly separated even when the proteins were run in the same well (lane 4). The two proteins appear to differ by 1 kD, more than may be anticipated based on the 5 amino acid difference in size. Nonetheless, this result is consistent with the 5′ extended transgene producing a larger translated protein, which is readily distinguishable from a codon mutated (Val to Met) 427 amino acid CD protein.

Northern analysis indicated that at the RNA level, there were no dramatic differences in the expression of transgenes in KB234, KB302, and KB571 cell lines, although KB302 cells tended to have less intense bands and KB234/14 cells appeared to express more CD RNA. The transcript sizes were different, 234/14 cells expressed a transcript of 2.0 kb, whereas, KB302 and KB570 cell lines expressed a transcript of 1.75 kb. This size difference may be due to different 3′ UTRs. KB571 and 302 cells have 38 bp of CD 3′ UTR and the 3′UTR of bovine GH to provide a polyA signal. We expect that the UTR for the RNA of 234/14 cells includes retroviral sequences.

Taken together the western and northern analyses demonstrate that the 5′ extended CD transgene used for the present Examples provide similar levels of protein and RNA expression as achieved with a bacterial CD gene mutated for mammalian expression (Mullen et al., Proc Natl Acad Sci USA 89:33-7, 1992). These results, furthermore, demonstrate that the present 5′ extended CD transgene (SEQ ID NO: 10) produces a novel CD protein (SEQ ID NO: 11) in mammalian cells that is larger than the 427 amino acid protein (SEQ ID NO: 12) of bacteria and the CD protein currently used as a suicide gene, (Mullen et al., Proc Natl Acad Sci USA 89:33-7, 1992 and Huber et al., Cancer Res 53:4619-26, 1993).

### Example 12

### Expression of CD in Human Cell Lines

The testing of plasmids in the cloning phase used rodent cell lines, because of the facility of transfection and expression in the lines used. We then also tested them in a human cell line which had been engineered to express human insulin, both to test the effect of the CD gene on cytotoxicity in a human cell line, and to evaluate the effect of cytotoxicity on secreted insulin. A second human cell line, HT1080, was selected to test the CD gene because this cell line is a line often used in tumorigenicity testing, and one in which a CD-cell-suicide system had been tested previously. In the HT1080 cells CD gene expression was reported to confer sensitivity to 5FC, however, at millimolar concentrations (Dachs, et al., Nature Medicine 3:515-20, 1997).

HT1080 cells and CD Expression, The HT1080 cell line was obtained from the ATCC and transfected with the WT CD gene of the present invention. A clone was isolated that had high expression of immunoreactive CD. In this clone the IC50 for 5FC was 110 μm. However, 5FC dominantly caused growth arrest with these cells and not killing. The growth arrested cells were replated to determine plating efficiency, and only about 30% of the cells plated, the remainder died, suggesting that the effective lethal concentration was lower than indicated by the IC50. Nonetheless, the WT CD gene of the present invention resulted in sensitivity to micromolar concentrations of 5FC with HT1080 cells. This represents toxicity at about 100-fold lower concentrations of 5FC than achieved by others with HT1080 cells (Dachs, et al., Nature Medicine 3:515-20, 1997).

The successful expression of WT-CD transgene was also achieved in human kidney cells −293 cells (IC50 55 μM), human colon tumor cells (IC50=45 μM), and CRL5816 cells (IC50=48 μM), demonstrating the ready expression of this extended CD construct in human cell lines.

ARPE-19 and CD Expression. Although ARPE-19 cells were transfected with CD gene, we were unable to isolate clones that were sensitive to 5FC (prodrug). The 5FU (toxic product of 5-FU and CD) cytotoxicity screen indicated that these cells are resistant to killing by 5FU. The purpose of the screen was to eliminate 5FU-resistant cells with the expectation that it would be difficult to use CD as a kill system in such cells. We conclude that lack of cytotoxicity with CD transgene and 5FC treatment in these cells is, at least in part, due to the insensitivity of these cells to 5FU cytotoxicity. The 5-FC and CD cell suicide safety system may not readily be an effective safety system with ARPE-19 cells.

CRL 5803 cells expressing BDNF and CD: The CRL 5803 cell line was transfected with BDNF and clones expressing BDNF were selected. These selected clones were expanded to provide cells for transfections with cytosine deaminase plasmid (CX014). Four CD gene transfection studies were performed with three different BDNF-expressing cell lines. For each cell line at least 24 resistant clones were picked and screened for sensitivity to 100 μM 5FC. The clones with the lowest survival in 5FC (as percent of control wells) were then expanded for dose-response studies with 5FC and 5FU. Cytotoxicity of 5FC was used as an indication of CD gene expression, and this screen provided 6-9 clones per transfection. The IC50 for 5FU indicated the maximum potential cytotoxicity of the prodrug, 5FC, in cells expressing sufficient levels of CD gene and protein.

The IC50 for 5-FU with the first cell line K3.4 was 8.5±0.3 μM, and with one of the transfections with the CD gene the 5FC IC50 was 79±9 μM, and with an independent second transfection the 5FC IC50 was 67±8 μM. The IC50 for 5FU with K3.3 and K3.31 cell lines was 5±1 and 9±1, respectively. The 5FC IC50s were 14±2 and 21±2 μM for K3.3 and K3.31, respectively.

Medium from these cell lines was assayed for BDNF expression by western blot, and the selected lines continued to secrete of BDNF.

These results indicate that the CRL 5803 cell line can effectively express both a secreted peptide, BDNF, and a cell suicide transgene, CD. The actual sensitivity of the resulting cell lines appear to be dependent on both the expression of CD and the sensitivity of the line to 5FU. Selection of clones transfected with a gene of interest for sensitivity to 5FU provide for CD-expressing clones more likely to have high sensitivity to 5FC.

### Example 13

### Stable Expression of Multiple Transgenes

The results obtained when co-expressing both a secreted peptide-BDNF, and a cytoplasmic protein—cytosine deaminase—indicated not only the ability of CRL 5803 cells to express two different transgenes, but the results indicated that both transgenes were stably maintained. Importantly, the maintained expression was in the absence of continual selection pressure, with selection antibiotics used in the post-transfection period only to isolate clones. Screening of clones and expansion of selected clones for further characterization were not done in the presence of antibiotic selection. It can be calculated readily that to expand from a single transfected cell to a pickable colony (≈10³ cells) requires about 10 population doublings. Typically, expansion for screening requires another 12 doublings, and then expansion for cryopreservation and for another round of transfection requires another 7 doublings. Therefore to transfect two transgenes involves ≈29 doublings, then selection for the second transgene and screening for the best expressors involves another >20 doublings. Thus, from the first transfection of BDNF in a single cell, to identifying, characterizing, and cryopreserving the progeny that also expresses the CD gene, all involves about 50 population doublings. Therefore, BDNF transgene expression was sustained through 50 population doublings, and CD through 20 or more doublings.

It was decided to further study the capability of BDNF transgene expression to be sustained in a clonal derivative of CRL 5803 cells by transfecting another transgene, selecting, and then screening for cells that express the third transgene. The third transgene selected was SEAP, with expression under the control of the UBq-promoter, and with NPT-resistance marker, also under the control of UBq promoter. The cells used for this transfection already expressed NPT-resistance transgene, and HPT-resistance transgene, both under SV40 promoter regulation. Therefore, the cells were selected under serum-free conditions and with elevated G418 antibiotic (1 mg/ml) to allow a growth advantage to cells expressing two NPT-transgenes. Three transfected plates were selected and each line of transfected cells was monitored for SEAP expression in the medium. The line with the highest SEAP expression was then trypsinized and replated in 10 cm dishes at clonal densities, and some trypsinized cells were suspended and briefly fixed with ice-cold 25% methanol (≈5 minutes), rinsed and then histochemically stained for alkaline phosphatase expression. A minor percentage of the cells, ≦10%, stained intensely for alkaline phosphatase indicating that a reasonably large number of colonies should be screened to identify the best expressing lines. The colonies of cells that grew in the dishes plated at clonal density were picked, and transferred to 48 well plates for expansion and screening for SEAP in the medium. The screening for SEAP expression involved medium collection (24 or 48 h samples), and then each well was assayed for viable cell mass with the resazurin assay, the ratio of SEAP activity to resazurin activity was then calculated to identify the lines that secreted the most SEAP/cell. The highest expressing lines were passaged to provide a well that could be stained for intracellular alkaline phosphatase (AlkPase), and a well that could be continuously expanded for each of the lines of interest.

The alkaline phosphatase histochemistry indicated that the lines selected for high expression did not stain uniformly for AlkPase activity. This suggested that the lines may not be entirely clonal, therefore the highest expressing lines were replated at clonal densities for recloning. The picking, and screening of clones for AlkPase in the medium and intracellular was repeated. A number of clones were found that uniformly expressed intracellular AlkPase activity. These clones were then expanded for cryopreservation, assay of BDNF in the medium, and for a further transfection. Western analysis was used to compare BDNF expression from the original cell line transfected with BDNF alone, and the clones that have been transfected with two additional genes, CD and SEAP. The result of this analysis demonstrated that BDNF transgene expression and protein production was sustained for ≧70 population doublings required for iterative transfection of BDNF, CD, and SEAP transgenes and to identify clonal isolates with each transfection.

An additional transfection was performed to examine the potential for the iterative engineering of four transgenes in CRL 5803 cells. A clonal line that expressed BDNF, CD, and SEAP was expanded for transfection to test a fourth transgene, and to test for retained expression of NPT [neomycin phosphotransferase]- and HPT [hygromycin-resistance transgenes. The cells were exposed to different doses of G418 (0.5 and 1 mg/ml), and to hygromycin (200 and 400 μg/ml) and cell growth measured to assess maintained SV40-promoter regulated transgene expression. The cells exhibited normal log-phase growth in the presence of either selection antibiotic. These results indicated the continued expression of resistance transgenes, and thus sustained transgene expression by SV40 promoter, for a minimum of 40-50 population doublings, in the absence of continuous selection.

The cells for transfection were plated in 10 cm dishes and then transfected with a new BDNF plasmid. This plasmid construct employed the UBq-promoter to control the expression of the second BDNF transgene, and for expression of the BSD [blasticidin S-deaminase]-resistance marker.

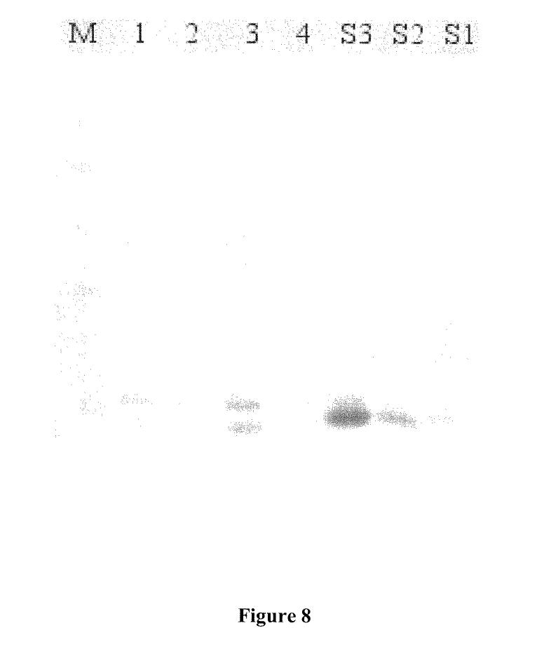

The transfected cells were selected for survival with 5 μg/ml of blasticidin (Blsd), and when all cells in the control plate (control=transfected with DNA without BSD trangene) were dead, the colonies in the BDNF-transfected plates were passaged as a polyclone. The polyclones were expanded and medium was collected for analysis of BDNF secreted by the polyclones. Expression of BDNF from these polyclones that were transfected with two BDNF transgenes was compared to that of the parental cell line that expressed BDNF as a result of a single transfection. Medium was collected from confluent 10 cm dishes for each of the cell lines, western analysis identified a transfected polyclone with an additional BDNF transgene under the control of UBq-promoter that released more BDNF into the medium than did cultures of parental cells with a single BDNF transgene (FIG. 8). The fact that the double-BDNF transgene transfection resulted in more BDNF secretion is consistent with sustained expression of the original (CMV-promoter driven) BDNF transgene, and additional BDNF expressed from the UBq-promoter driven BDNF transgene in this polyclone. The polyclone was also examined for BDNF secretion when under selective pressures of 5, 15, and 30 μg/ml of Blsd, release appeared to be slightly reduced by higher concentrations of Blsd selection drug, and all secreted more than the parental cell line with one BDNF transgene (FIG. 8).This polyclone represents CRL 5803 cells that have been engineered to express seven different transgenes (NPT, HPT, BSD, BDNF×2, CD, and SEAP), and have sustained expression of a BDNF transgene for ≧80 population doublings. Therefore, CRL 5803 cells have been demonstrated to be robust candidates for therapeutic transgene expression, with ready expression by either CMV-, RSV-, SV40-, or UBq-promoters, and sustained transgene expression for at least 80 generations without the need for continued selection pressure as is often required for high-level expression.

The cell line created with this multiple engineering is designated K71.p and has been deposited with DSMZ under the Budapest Treaty. The accession number given by the International Depository Authority is “DSM ACC2730”.

### Example 14

### Demonstration of Cell Function in Encapsulated Devices

A. Growth of Cells in Vitro

Initial studies were performed to show the growth and function of the encapsulated cells in vitro.

Comparative Assay of Cell Size, Packed Volume and Function:

The cell lines used were CCL205 a human lung fibroblast cell line, CRL1440 a human myoblastoma cell line, CRL 5803 a human lung neuroendocrine cell line, and ARPE-19, a human retinal pigmented-epithelium cell line. These lines were transfected with the BSD-expression plasmid (to facilitate recovery from animals by killing mouse host cells with the drug blasticidin, to which these transfected cells are resistant). The cells were removed from plates for these studies or for passage with 0.05% trypsin (gamma-irradiated), 0.05% EDTA in calcium and magnesium-free phosphate-buffered saline (PBS).

Size and Volume: Trypsinized cells were used to measure cell diameters. The rounded, trypsinized cells were photographed with a Canon digital camera attached to a Leica DM inverted microscope using a 40× objective. A stage micrometer (1 mm in 100 gradations, Edmund Scientific) was photographed with the same objective for each cell line series. The digital photographs were transferred to a photograph database (iPhoto, Apple Computer, Cupertino, Calif.) and printed with a Hewlett-Packard printer (Color Laserjet 4600dn) at a final magnification of ≈1000×. Perpendicular diameters of 50-150 cells were measured, and averages, standard deviations, and coefficients of variation were calculated. A sufficient number of cells were measured for each line such that the coefficient of variation for the cell diameter measurement was less than 20%, (with the exception of CCL205, which was found to have two distinct size populations). The magnification factor was calculated by measuring the accompanying photograph of the stage micrometer, and the cell diameters, in μm, were calculated. Individual cell volumes were then calculated using the average diameter for each cell line, assuming an approximately spherical geometry.

Correlation of Cell Volume, Cell Activity, and Cellular Protein: Cells were grown in T75 flasks and trysinized to provide cells in suspension. The suspended cells were rinsed twice in PBS to remove trypsin and centrifuged in conical, graduated microcentrifuge tubes 1 minute at 500×g. The pellet volume was estimated from the graduated markings on the tube (usually ≈50 μl of cells, and an equal volume of PBS was added to suspend the pellet. The total volume of the suspended pellet was then confirmed with a variable volume micropipette (Finnpipettes, Thermoelectron). The cells were then suspended at 3 μl-pellet volume/ml of medium.

The relationship between functional mass, pellet volume, and cellular protein was measured with CRL 5803 cells. CRL 5803 cells (facility-PD5), 45 μl pellet, were suspended in 11 ml, and used to measure functional resazurin activity. The cells were set up at 25, 50, 100, 200, & 400 μl of suspension in total of 500 μl medium in wells of a 48 well plate, and 500 μl of 80 μM resazurin was added to each well. This provided a standard curve with 0.1, 0.2, 0.4, 0.8 and 1.6 μl of cell pellet/well. The same cell suspension was used to measure cellular protein. A portion of the total suspension, 6.35 ml, was pelleted at 500×g for 1 minute. The pellet was washed with 14 ml PBS and frozen in 1 ml 0.9% NaCl for measurement of total protein. The cell pellet was frozen and thawed three times to lyse cells before measurement of protein. The protein standard was bovine albumin (Sigma).

The functional cell mass was then compared using the dye resazurin. The production of resorufin by 0.5 ml of cell suspension mixed with 0.5 ml of 80 μM resazurin, was measured kinetically in a fluorimetric plate reader (SpectraMax M2, Molecular Devices; 557 nm excitation, 590 nm emission). The functional activity was expressed as relative fluorescent units (RFU), based on the calculated Vmax (an output variable from the SoftMax Pro software). The functional activity of the four cells lines was then compared (RFU/μl pellet volume).

Cell Diameters and Volume: As shown in table 2, the cell lines examined ranged from 14-18 μm in diameter, and an associated more than two-fold range in individual cell volume. Note that with these values the number of cells/cm³ or cells/ml can be calculated, and the results of 3-7×10⁸ cells/cm³ are consistent with values for cells at tissue density in the body. These data indicate that a micro-bioreactor device with a 4.5 μl volume (sized for rabbit and monkey studies) could contain 1 million of the largest cells and 3 million of the smallest. Assuming that all cells produce similar amounts of transgenic protein per cell, the CRL1440 and ARPE-derived cell lines should be the most efficacious cells in devices.

| | | | | Cells in 4.5 μl | | |
|---|---|---|---|---|---|---|
| | DIAMETER | VOLUME, | VOLUME | device | Cells/ml | |
| | μm | μm3 | 10⁻⁶ μl | millions | millions | n |
| CCL205 | 17.5 ± 5.3 | 3657 ± 4579 | 3.7 ± 4.6 | 1.2 | 270 | 41 |
| (K36.3p) | | | | | | |
| CRL1440 | 14.4 ± 1.7 | 1633 ± 561 | 1.6 ± 0.6 | 2.8 | 610 | 26 |
| (K21.p) | | | | | | |
| CRL5803 | 17.8 ± 2.7 | 3131 ± 1479 | 3.1 ± 1.2 | 1.4 | 320 | 143 |
| (K29.p) | | | | | | |
| ARPE-19 | 13.6 ± 2.6 | 1445 ± 797 | 1.4 ± 0.8 | 3.1 | 690 | 66 |
| (K14.p) | | | | | | |

Cell Volume and Activity: There was a good correlation (r2>0.99) between the pellet volume and the activity of the cells with the resazurin assay with CRL 5803-derived cells. Based on calculations made using the fitted line, 3 μl (about 1 million cells by volume) of packed pellet would have an activity of 8000 RFUs in a short-term assay (15 minutes), and the capacity to detect less than 50000 cells in a 48 well plate. (Note that this calibration must be done for each different plate-size used, as volumes are different and light path differs with different well sizes).

Aliquots of viable cells for each cell line of the same cell volume (2.3 μl pellet) in 1 ml of a 24 well plate, exhibited similar functional activities: ARPE19/K14.9=8352 RFU; CRL5803/K29.p=8898 RFUs; CRL1440/K21.p=9224 RFUs; CCL205/K36.3.p=9865 RFUs. These results indicate that the functional capacity of these cells was more closely related to the pellet volume than the cell number represented by that volume. In addition, the results suggest that total cell volume can be estimated from the same standard curve (given that all conditions are constant). The health of each of these different cell lines was demonstrated by following the growth rate for at least one week.

Cell Volume and Protein: The packed cell volume (pellet volume) and cell protein were also highly correlated (r2>0.99), with about 250 μg of protein for each 3 μl volume, or about 250 μg/million CRL 5803-derived cells.

B. Growth of Cells in Devices

Cells from each of the four human cell lines were trypsinized, washed and concentrated so that approximately 0.5 μl packed-cell volume was loaded into a cell-encapsulating microbioreactor device using a 0.6 ml volume (medium flows out through the device surface membrane, leaving behind the live cells). The sealed devices were then cultured, one per well, in 24-well plates. Parallel cultures were plated in 24 well plates to establish monolayer cultures. The growth of cells in devices and cells in monolayers were measured continuously at 1-2 day intervals using the resazurin assay. The activity data from the resazurin assay was converted into volume units based on a standard curve of cells in suspension. Data from seeding until plateau phase of growth was used to determine the doubling times for each cell line, and to determine the plateau phase cell volume attained by each cell line in the devices. The growth of monolayer cultures provided data to indicate the health of the cells used to seed the devices.

If the cell volume is the dominant determinant of the capacity of cells in devices to maintain a functional cell activity, then all cell lines would be expected to achieve a similar total functional activity in the devices. If cell number determines the capacity of devices, one might expect that smaller cells would achieve more optimal packing efficiencies, more cells in the device volume, and achieve higher functional volumes. The results indicated that the four cell lines do not achieve the same peak or steady-state densities. The lines K14.p (ARPE-19-derived) and K36.3p (CCL205-derived) represent the cells with the smaller and the largest cellular volumes, yet the achieved densities (functional volume/device volume) of these cells in the first weeks are the lowest. All four of the cell lines reached plateau phase of growth in the first five days of culture in devices. Doubling times (n≧3 devices) were 61±6 h for K14.p/ARPE-19, 47±4 h for K21.p/CRL1440, 70±10 h for K29.p/CRL5803, 46±7 h for K36.p/CCL205 cells. The typical doubling times for cell monolayers with each of these cell lines is about 30 h or less, so these cell lines exhibit a modest (≈50% for CRL1440 and CCL205) to pronounced (100% for ARPE-19 and CRL 5803) increase in doubling times when growing inside the devices. The functional cell volumes, as calculated in plateau phase, represented 30±4%, 35±8%, 57±4%, and 60±8% of the device volume for K14.p/ARPE-19, K36.p/CCL205, K29.p/CRL5803, and K21.p/CRL1440 cells respectively. The plateau phase cell mass was sustained for at least 7 days in vitro. These results indicate that the CRL 5803- and CRL1440-derived cells most efficiently fill the device in vitro.

In subsequent studies it was found that after 2 or more months the intermediate-sized cell, derived from CRL 5803, sustains the highest functional capacity in devices.

C. Survival and Function in Vivo

Sustained Function in vivo: Comparison of Cell Lines: Three to four devices for each cell line were loaded with cells, and while in late log phase of growth (near plateau of viable cells in devices), the cells in devices were implanted subcutaneously in the backs of nude mice. Nude mice were used because these athymic animals provide a model in which much of the immune system involved in transplant rejection is lacking. As a result the in vivo survival of human cells in the cell-encapsulating device context is more a reflection of the capacity of the device and the host organism to provide for appropriate nutrition supporting cellular function. It is likely also a reflection of the cell type to survive while the surgical site heals, re-estabishing the vascular supply of the implant site. Nude mice are anticipated to provide an indication of cell and device function for the allograft therapeutic setting—human cells in humans.

The implant sites healed, with normal appearance of the implant site by two weeks. The devices were removed after 1 month, and returned to culture in 24 well plates with Blsd selection. Devices of similar dimensions, but that had not been seeded with cells, were also cultured in Blsd selection. These empty devices also had host cells attached; these non-seeded devices provided a means to follow the death of host cells. Host cells declined in function over 5 days such that after 5 days the host cell contribution to a viable cell signal was not measurable. The evaluation of the devices loaded with different cell types was based on viable cells present in devices after 5 days of selection.

Cells derived from either the CCL205 line or the CRL1440 line were difficult to maintain in vitro in plateau phase. It was not possible to detect viable cells after explant with devices containing CCL205-derived cells. With devices seeded with CRL1440-derived cells the outcome was inconsistent. One device appeared to have maintained viable cell mass, or increased slightly in vivo. The second in vivo device did not contain a detectable number of viable cells after explant.

Cells derived from ARPE-19 are sustainable in devices in vitro, however, the function of cells in devices after explant was variable. Explanted devices contained viable cells, one with essentially the same functional cell mass as when implanted, but the second device had 30% loss from pre-implant values. In the one month follow-up in culture the first device maintained cell function, while the second device exhibited a slow decline in function.

Cells derived from CRL 5803 exhibited a consistent response after explant and return to culture. All three devices had essentially the same function (representing 50-60% of device volume) after explant and for the month of follow-up culture, relative to that measured before implanting into mice.

These results indicate that in the context of maintained cell function both in vitro and after one month in an animal in a device, the most robust cells are the CRL 5803-derived cells. In addition, the results indicate that the UBq promoter function was not silenced in CRL 5803 cells after one month in vivo. Had the UBq promoter been silenced, the CRL 5803 would not have survived Blsd selection of explant culture. We can not rule out that UBq-promoter stability was a factor in the poorer functions of the other cell lines with the Blsd selection in explant culture.

It is possible that with the other cell lines appropriate conditions could be developed to provide more consistent performance in vitro. Consistent in vitro function may then interpret into more consistent in vivo function. However, because the CRL 5803-derived cells were already found to be more robust in terms of engineering and other properties, (as described in earlier Examples) the similar finding of robust in vivo performance, brought us to center subsequent device studies on this cell line.

These nude mouse-device implant studies illustrated the capacity of plateau-phase CRL 5803 cells in devices in an immunologically benign implant model. We then examined whether the robust nature of CRL 5803 cells includes the capacity to grow in devices during implantation, and whether CRL 5803 cells are robust in the context of a normal (xenogeneic) immune response.

### Example 15

### CRL 5803 Implants in Immune-Compromised and Immune-Competent Mice

CRL 5803-derived cells that expressed both the BSD transgene under control of the UBq-promoter and a CD transgene (K29.p) were used for the studies of growth and in vivo studies in the context of a normal immune system. Six devices were seeded and cultured in low serum. In order to provide a functional cell mass that represented ≈30% fill of device volume the devices were switched to medium with no serum for three days and then to a low nutrient medium (M7) with no serum or protein supplement for three days in the presence of 1 μg/ml blasticidin. Protein-free culture was used to reduce immunogenic bovine serum proteins associated with devices. Blasticidin was used in these 3 days to measure functional activity pre-implant, for later comparison with explant data. Four devices were implanted subcutaneously on the back of mice, either in normal or nude-immunocompromised BALB/C. Two devices were maintained in vitro, and these in vitro controls were returned to normal culture medium.

The devices were explanted from nude mice, and normal mice 2.5 weeks after implantation. The explanted devices were cultured in blasticidin (Blsd; 2 μg/ml) selection antibiotic to kill host cells on the devices, while selecting for cells that continued to express the selectable marker. Assessment of viable cells in the device was not started until after 5 days of treatment with Blsd selection. Devices explanted from normal mice had a marked reduction in viable cells in the devices, to 25% of pre-implant levels. In contrast, devices that were recovered from immuno-compromised nude mice had no reduction, in fact the number of viable cells indicated that cells in these devices had more than doubled. The cells in devices recovered from normal mice regrew after explant to 75% of pre-implant by two weeks of explant culture, and recovered to 100% (50-60% of device volume) with one month of culture. The cells in devices recovered from nude mice maintained the increase in viable cells achieved in vivo, representing 50-60% of device volume, for the one month follow-up of in vitro culture. The in vitro control devices, in parallel with the devices explanted from nude mice, grew to and maintained a cell volume that represented 50-60% of the device volume.

These results indicate that CRL 5803 cells were able to grow in devices, in vivo, in the context of the compromised immune system of the nude mouse. The CRL 5803-derived cells did not retain functional cell mass in the context of mice with a normal immune system, indicating the susceptibility of these cells to a xenogeneic immune environment of the normal mouse. In addition, the fact that the cells in devices were immediately resistant to Blsd, on return to culture, is consistent with retained function of the ubiquitin-promoter in vivo. The growth of cells in animals with an impaired rejection response, suggested the possibility of using immunosuppression to ensure initial survival of CRL 5803 cells in larger animal models, where there are no readily available immunodeficient models.

While these in vivo studies indicated that both the CRL 5803 cells and the promoter used were functionally robust in the device and in the in vivo context, this was for a relatively short duration. Longer term studies were also performed to confirm that both the UBq-promoter in the context of CRL 5803 cells, and CRL 5803 cells in the context of the BioD device are functionally robust in vivo.

### Example 16

### Long Term Survival of Implants in Mice

Devices with CRL 5803-derived cells-K29.p—that had been implanted for 2.5 weeks and then evaluated for one month in culture were used to evaluate long-term survival. Devices with ARPE-19-derived cells (K14.p) expressing BSD transgene were also included in this study as a reference cell for in vivo stability; (only three devices were available for this cell line because one device had failed after return to in vitro culture). Each nude mouse was implanted subcutaneously with two devices: one device with K29.p cells (right side), the second with K14.p cells (left side). At the time of implant both the K29.p and the K14.p devices already had a lifespan of 3 months. One device for each line was maintained in vitro.

Devices were explanted after almost 3 months implantation (114 days). The explanted devices were returned to culture in Blsd selection. Only one of the two K14.p devices contained functional cells after selection, it is unknown when this device actually failed in vivo. The in vitro control device for K14.p began to fail one month after implantation, and although maintained in culture until the in vivo devices were removed from mice, the in vitro control device did not recover. The K14.p devices had three failures: one did not recover after the first period in a nude mouse (≦69 days), another failed in vitro (120 d), the third did not recover after a second period in a nude mouse (≦218 d). The fourth device survived two periods in nude mice and was recovered to culture and survived in vitro until the study was terminated at >350 days, with a functional mass identical to that found after the first in vivo period (∓9 weeks). The average lifespan with K14.p devices was calculated as 192 days.

In this study all K29.p devices survived until the study was stopped, both the two devices with two periods in nude mice and the in vitro controls. The function of these cells was actually higher in the week leading to the one year survival, than when recovered from the first in vivo period (≈9 weeks). The average lifespan with K29.p devices was then >350 days.

The failure of K14.p cells in devices does not represent an inherent cellular lifespan. It is possible to maintain ARPE-19 cells as a confluent monolayer for periods of at least one year, just as one of four devices survived up to a year. It is not clear why some devices with ARPE-19-derived cells fail, nor why survivals are so varied, although the findings are consistent with literature reports (Thanos et al Tiss Eng 10:1617, 2004). Two cell lines that were derived from ARPE-19 were reported to exhibit impaired function in devices. An 80% initial loss of function was associated with adaptation to the device (decrease from 800 to 153 ng/million cells for one line, and 250 to 50 ng/million in another), a second, more gradual loss of function was observed after implantation. The loss of function in a device in vivo was used to calculate the functional half-life, this is the time in vivo required to reduce function 50%. The two different cell lines had different half-lives, of ≈2.5 months and ≈6 months (Thanos et al. Tiss Eng 10:1617, 2004). It is possible that the present observations made in the context of a different device reflect inherent longevity issues with ARPE-19 cells in devices. The variablilty of survival in devices observed in the present studies may reflect the non-clonal nature of the cells studied, particularly because the ARPE-19 cells are known to produce clonal lines with differing longevity (Thanos et al., 2004).

The reason that other cell lines function poorly in the current devices is unknown. However, the poor function of other cells provides a contrast to engineered CRL 5803 cells. The consistent survival of CRL 5803-derived cell lines, such as K29.p, in devices for ≧1 year illustrate the robust nature of these cells in the context of the device.

### Example 17

### Survival of Implants in Rabbit Brain

In order to examine the function of cells in a device in the context of the brain it requires a more complex system than can be achieved in vitro where known components of CSF can be examined. Transplanted cells, are not actually exposed to CSF, rather nutrients would be supplied by what is available in the intercellular fluid of the brain parenchyma. In addition, cells in devices that are sized for site-specific therapy of the human brain can not be appropriately tested in a rat or mouse brain, where the sites of interest are dwarfed by such a device size. Therefore, our initial studies of cell and device function in the brain used rabbits as an experimental model.

The cells were implanted into the rabbit brain using a dual component device. The first component is an implantible housing that integrates with host tissue (the rabbit brain) into which the second component—a cell-containing housing (described as “device” in the previous in vitro studies) is inserted and the implantible housing sealed. The implantible housing provides for recovery of the cell component without disrupting the brain-implantible housing interaction. With such an implant system the function of implanted cells can be evaluated immediately upon device removal without the interference of host cells.

Cells: The cell line used in these studies, derived from CRL 5803 cells, have the capacity to survive in a model cerebrospinal fluid (CSF), have low serum-requirement for growth, are sensitive to 5-FU, a chemotherapeutic, and have the ability to synthesize, process, and release transgenic secretory proteins without density-associated down-regulation.

The CRL 5803 human cell line was engineered to express both a Histidine-tagged neurotrophic factor (BDNF), and an enzyme that provides for selective killing of the engineered cells, (cytosine deaminase). Both the BDNF, and the cytosine deaminase transgenes are under the regulatory control of a CMV promoter. The expression of selectable markers are regulated by the SV40 promoter.

This cell line, K44.20, with BDNF and CD transgenes was also transfected with SEAP to confer expression of this enzyme under control of the ubiquitin promoter, producing the K63A.p cell-line. The SEAP enzyme can be detected with a non-invasive and sensitive fluorescent assay for alkaline phosphatase (AlkPase). The cell line produced was not clonal for SEAP expression, with ≦10% of the cells exhibiting intense stain for AlkPase activity. We considered the possibility that the SEAP would be recognized by the rabbit immune system and result in an inflammatory response at the device implantation site, therefore cells with and without SEAP were implanted.

Plasmids: The SEAP plasmid was D1-2, the BDNF plasmid was # B1-1, and the CD plasmid was # C1-1.

Chemicals: Selection drugs were G418 and hygromycin (Invitrogen). Resazurin, umbelliferyl phosphate and buffers for SEAP assay, as well as reagents for histochemical demonstration of SEAP were obtained from Sigma Aldrich (St. Louis Mo.). See earlier Examples for assay details.

Rabbits: Four male, New Zealand white rabbits, of 2-3 kg body weight were used for these implant studies. Two rabbits were immunosuppressed with cyclosporine, 10 mg/kg, for the duration of implant, the other two rabbits were not treated with immunosuppressants.

Devices: The devices used were a two component design. The outer cylinder was designed to ensheath a cell-loaded device, and the host surface designed to integrate into the brain parenchyma (U.S. Pat. No. 5,913,998). The normal cell-encapsulation device was seeded with cells, sealed, and then immediately inserted into the outer ensheathing component. The ensheathing component was then sealed and the entire assembly was placed into culture. The K44.20 cells were cultured for ≈4 weeks, while the K63A.p cells were cultured for 3-4 days.

Transport of Devices: Each device was transported to the animal facility (4-6 h) in 50 ml of medium in a 50 ml conical centrifuge tube (Falcon Labware). The medium was a buffered derivative of M7 medium. The tubes were packaged in styrofoam containers with sealed containers of water warmed to 25° C., (≈400 ml total), to provide a heat sink for maintenance of the temperature between 20-25° C. The ability of the styrofoam container and “heat sinks” to maintain the temperature at 20-25° C. for >24 hours had been established in earlier trials.

Implantation of Devices: Each rabbit received bilateral device implants. The cells for the device implanted on the right side expressed BDNF and CD transgenes, K44.20, a clonal derivative of engineered CRL 5803 cells. The device implanted on the left side contained K63A.p cells that were derived from K44.20 cells by transfection with a SEAP-expressing plasmid. The rabbits were anesthetized, stabilized in a stereotactic frame providing for accurate implantation into the basal ganglia.

Recovery of Devices: Devices were recovered from anesthetized rabbits that were stabilized in a stereotaxic frame. The bone cement was carefully cut away, then the bone wax removed to reveal the exposed, sealed ends of the two devices. The tips protruded from the brain and dura mater 1-2 mm, with the dura encircling and attached to the device. The bone wax may have prevented the dura from attaching to the 1-2 mm of protruding device. A forcep, stabilized on the cranium adjacent to the opening, was used to hold the protruding tip, while an iridectomy scissor was used to carefully cut the outer ensheatment of the two component device. In this way, a sealed end of the cell-encapsulation device was exposed. A second forcep was used to slowly retract the cell-encapsulation device from the ensheathment component. The first forcep, in combination with the dural attachment, stabilized the implanted ensheathment device so that this portion of the device system remained attached in the brain parenchyma.

Retrieval Assessment and Transport: The retrieved cell-encapsulation components were placed into medium (containing selection antibiotics) at room temperature until all devices were retrieved. The devices were then transferred to an adjacent cell culture facility and assayed for function using the resazurin assay. Upon completion of the functional assay, the devices were prepared for transport (as described above) for return to the home cell culture laboratory. The devices were then reassayed to ensure maintained function after transport, and returned to culture to monitor post-explant function.

Fixation and Sectioning of Brain: The ensheathment component of the devices were fixed in situ, by removing the rabbit brain and trimming away much of the surrounding brain, leaving 5-10 mm of parenchyma attached to the device. This block of tissue was immersion-fixed in paraformaldehyde, frozen and 50 μm sections prepared.

Results

Impact of Brain Environment: The impact of the brain environment on the cells in a device was evaluated using an assay of functional cell activity. The minimally fluorescent molecule resazurin is converted to resorufin by enzymes of a living cell, this production of fluorescent resorufin by cells is proportional to viable cells and was used to evaluate cell function. Cell function was measured at the time of implant and of explant, and during in vitro culture in medium with G418 after removal from the rabbit brain. Culture in G418 serves to indicate the retained activity of the promoter that drives the selection-marker gene in these cells.

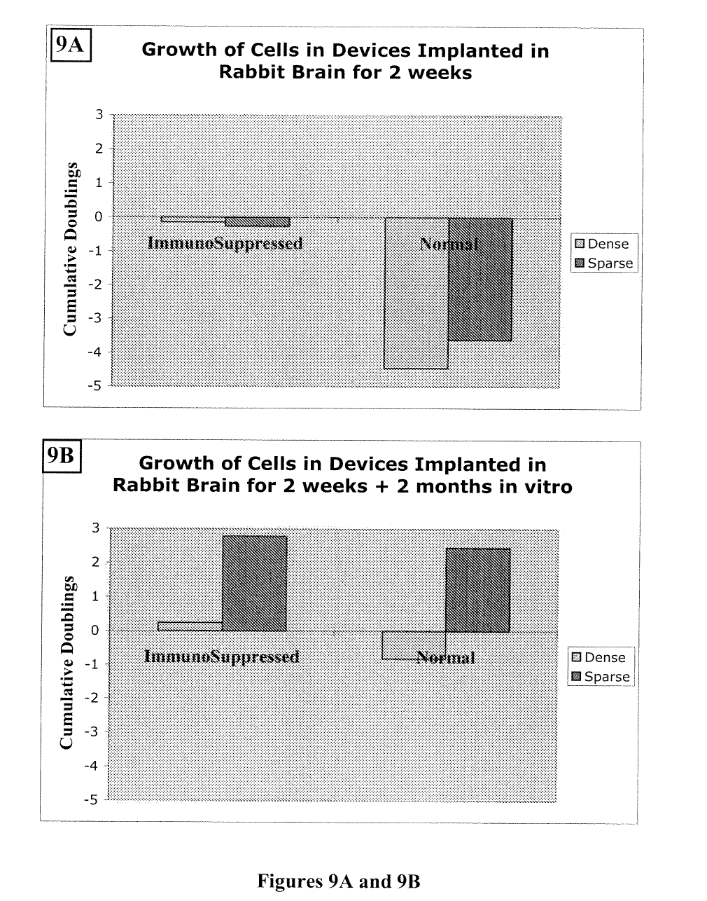

FIG. 9A illustrates the impact of the brain on cells housed in a device and implanted for 2 weeks. The bars to the left indicate the growth of cells that were either at near-maximal, (those not expressing SEAP), or sparse densities, (expressing SEAP), in devices when implanted in rabbits immunosuppressed with cyclosporine (10 μg/kg). The bars to the right indicate similar devices that were implanted into normal rabbits (no immunosuppression). With immunosuppressed rabbits the cells in the device did not show any net growth in the 2 weeks in the rabbit brain, with indications of slight cell loss. In contrast, cells in devices implanted into normal (no immunosuppression) rabbit brain did not grow, but actually lost considerable functional cell mass—as indicated by negative cell “doublings”.

Although cells in these devices were unable to grow in vivo, cells grew when removed from the rabbit and were placed in cell culture, FIG. 9B. The most dramatic growth was by cells that were in sparsely seeded devices. Relative to pre-implantation, these cells doubled 2 to 2.5 times with return to cell culture medium in the presence of G418, whether the devices had been in normal or immunosuppressed rabbits. The cells in densely seeded devices did not grow as dramatically, although there was a return toward pre-implantation number of cells in devices from both immunosuppressed and normal rabbits. However, the device in which cells had decreased functional mass after implantation in the normal (non-immunosuppressed) rabbit brain did not grow enough to recover the cell mass to that present at the time the device was implanted into the normal rabbit brain.

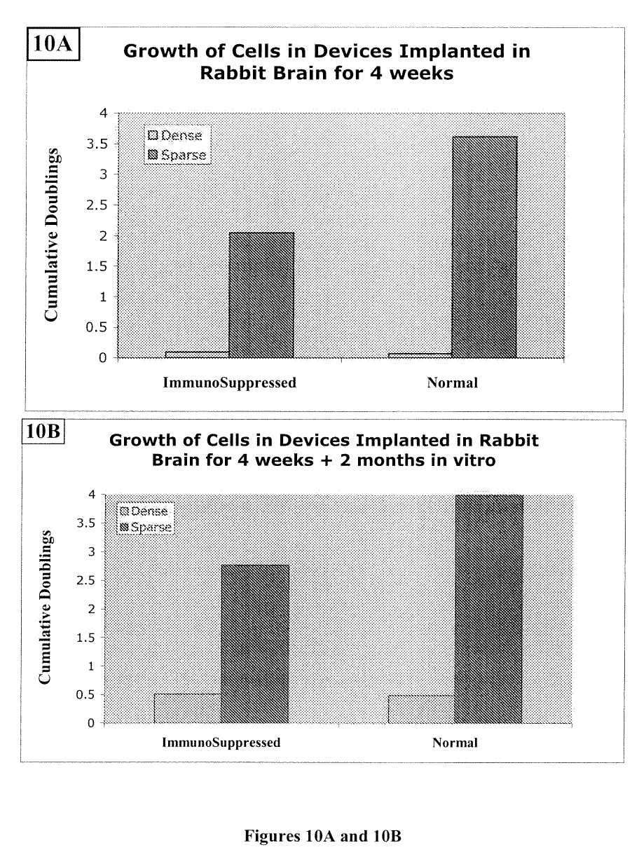

In contrast to cells implanted into rabbit brains for 2 weeks, those implanted in the brain for 4 weeks all grew whether in the brain of an immunosuppressed rabbit or in a normal rabbit (FIG. 10A). The growth potential of these cells in the brain in a device is best illustrated by the growth of cells at a low density when implanted. These cells doubled 2-3 times in the 4 weeks with or without immunosuppression. This indicates doubling times of 9-16 days in the rabbit brain. These cells also grew more slowly as monolayers when in a salt solution formulated to approximate nutrient (glucose and amino acids), and protein levels found in the brain. Those model studies suggested that with dense monolayers doubling times were shifted from about 2.7 days with medium to >1 month with model CSF. It would appear that the rabbit brain has better growth promoting properties than a simulated CSF (9-16 days in the brain, rather than 30 day doubling times in modeled CSF).

Cells in devices implanted for four weeks in the rabbit brain apparently grew to essentially device-filling density while in vivo. This is further indicated by only slight cell expansion that occurred with an additional 2 months of in vitro culture following removal from the rabbit brain (FIG. 10B).

These device implantation results indicate that, in the context of the dual component implantable device, human cells can not only survive, but also maintain function when implanted in the brain of rabbits. Function was demonstrated 1) by the recovery of active cells in devices at explant, and 2) by the growth of cells when in the rabbit brain for 1 month. 3) Sustained function for several weeks after explant. Cells seeded at near maximal density in devices maintained the functional cell mass in the devices, whereas those seeded at about 20% of capacity grew to essentially fill the devices.

The results suggest that in the initial weeks after implantation, the cells benefit from immunosuppression. However, the net growth of cells without immunosuppression for 4 weeks in a normal rabbit suggest that after the healing of the surgical site, the cells can be nourished adequately to provide for growth. We speculate that the deleterious impact of brain implantation in the initial weeks is due to a number of variables, among which are inadequate nutrition during the healing process, and the action of host immune cells during the healing of the implant site.

The survival of the cells in the present devices and in the context of the rabbit brain with and without immunosuppression could not be predicted based on the device design. The material of these devices does not provide a specific molecular sieving function, as the structure of the ePTFE wall of the device predominantly acts to prevent the movement of cells—both host (from entering) and implanted cells (from escaping). We infer that in the brain, lethal effects of the host can be minimized when direct cellular contact between the host and the therapeutic cells inside the device is prevented.

The devices, when returned to cell culture in vitro, were cultured in medium containing the selectable marker G418. Only if the cells continue to express the selectable marker enzyme neomycin phosphotransferase (NPT) will the cells survive and grow in vitro. Thus, an additional indication of this study is that, at a minimum, the SV40-promoter, the regulatory element used to drive transcription of NPT gene (in the B1-1 construct), remains active in these cells in the milieu of the brain.

Similarly, the transfection of some of the cells implanted (devices implanted on the left side of the rabbit brain) with secreted alkaline phosphatase (SEAP) provided a marker of the activity of the ubiquitin promoter. SEAP was detected in medium from devices in the initial days after removal from the rabbit brain. However, because only about 10% of the cells actually expressed SEAP, the SEAP levels could not be quantified reliably in devices with low cell numbers.

There is evidence to suggest that protein expression assessed in vitro or in regions other than the brain may not reliably indicate expression levels achieved in the CNS. In one study (Zermansky, Mol Ther. 4:490-8, 2001) the viral-directed expression of one transgene (TKase) was widespread and long-lasting, while a different transgene (B-Gal) provided at the same time, was neither as well-expressed, nor did it have similar longevity in the brain. The present results with clonally-derived cells housed in a device provide indications that more than one transgene continued to be expressed while in the CNS (NPT and SEAP).

### Example 18

### Survival and Function in Monkey Brain

The macaque monkey is known as an old-world monkey. “Old-world” monkeys do not attach alpha-galactosyl moieties on cell surface markers, making these primates more similar to humans. The presence of these moieties is an important component of acute host-vs-graft response towards a xenogeneic implant. Since the device does protect against direct cell-to-cell contact, this mechanism of rejection is masked. However, “Old-world” monkeys such as the macaque represent the best animal model in which to conduct studies not complicated by immunosuppressants.

This study was designed to evaluate survivability of devices with human cells, (xenogeneic implant), in the context of the brain parenchyma, specifically the basal ganglia of the primate brain. A primate model is considered to more closely resemble the human brain than other animal models. In addition, primate models of Parkinson's disease represent a movement disorder model with movement disorders most like PD in man.

Animals: The experiment used a male cynomolgus monkeys (Macaca fascicularis; age=4 years; weight=3.5 kg).

Cells and Devices: The two cell lines used were engineered derivatives of CRL 5803. One cell line, K3.31, expressed the BDNF transgene (and the NEO selection gene). The second line was created by engineering K3.31 cells to express the cell suicide transgene cytosine deaminase (CD), the K44.20 clone was selected based on the toxic efficiency of 5-FC. Cell suicide genes have been reported to be inflammatory in the brain, the use of cells with and without a cell suicide transgene provide a potential means to examine inflammatory response.

Surgical Implantation: Devices were stereotactically implanted into the right and left basal ganglia. Two cell- containing double-component devices were implanted vertically, one on each side of the brain in the frontal plane at ear bars +20 mm, lateral 8 mm. The left device had cells expressing BDNF and CD, cells in the right device expressed only BDNF (not CD). An empty, control device was implanted vertically on the left side of the brain in the frontal plane at ear bars +12 mm, lateral 11 mm. The left, non-loaded device was left empty as a control.

Explant: Two weeks after implant the animal was anaesthetised and the cell-housing component of the devices was recovered and immediately assayed for function with the resazurin assay.

Results

This study involved implanting 3 ensheathed devices (two-component devices). Two devices contained cells and were implanted bilaterally in the more frontal region of the basal ganglia. An empty device was implanted in the more occipital region of the basal ganglia. The cells were from a line that expressed only BDNF, or the progeny cell line that expressed BDNF and was also engineered to express a cell suicide gene (cytosine deaminase). The latter line was implanted in the left hemisphere, and the former in the right. After stereotaxic implant in the brain it was noted that the seal for the outer component of the right cell-containing device was damaged, but the two-component device was not removed. The cranium was closed and the devices remained in the monkey for 2 weeks. After two weeks the cell containing devices were removed from the monkey brain, recovered to culture, and assayed for functional activity.

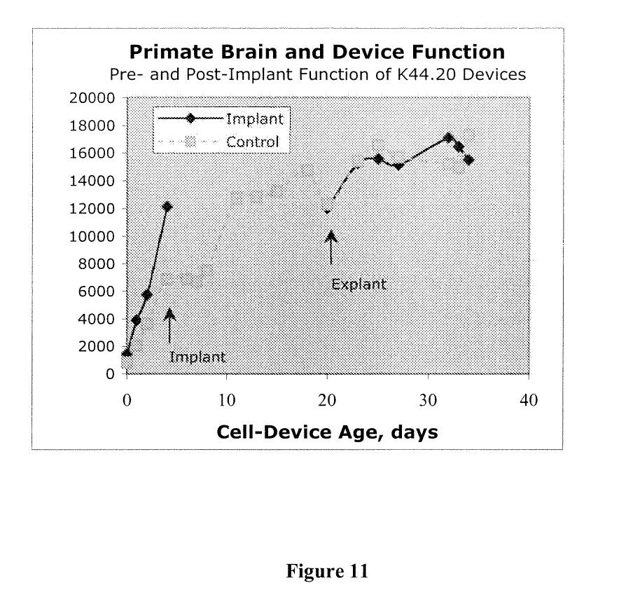

The acute functional activity (first hour after removal) of the left device was indistinguishable from the activity immediately prior to implant. The right device included what appeared to be host cells inside the defective sheath, and activity was greatly reduced with this device. Kinetic assessment of the devices in the laboratory 16 hours later indicated that the device in the defective sheath had lost most functional/viable cells, while the cells from the intact two-component device, (expressing BDNF and CD), were completely recovered (FIG. 11), and comparable to a cell-seeded control device that was transported to the primate facility and returned to the culture laboratory, but otherwise maintained in culture. In addition, for both in vitro and explanted devices the cells grew to maximal density in the devices after return to culture in the presence of G418.

In the context of this more concordant xenograft—human cells in the old-world macaque monkey brain (non-immunosuppressed)—implanted cells in devices were recovered quantitatively, after two weeks in the basal ganglia of the primate brain. The full functional recovery of the candidate cells in loaded and ensheathed devices (two-component system) parallels the recovery from the brain of immunosuppressed rabbits. This recovery includes shipping to and from the monkey facility for a total of about 3 days, plus 2 weeks in the monkey brain. No loss of cells was seen in the device through these procedures and the time in the brain, supporting the consideration that the monkey is an appropriate model animal to test our human cells and further suggests the possibility that immunosuppression is not required in future monkey studies. In addition, immunoreactive BDNF was captured on 96 well HIS-tag affinity plates (Sigma Chemicals), and the level of output appeared to match that of in vitro controls.

These studies in the primate brain confirm the results of studies in the rabbit brain, and support the conclusion that engineered CRL 5803 cells in devices represent a relevant “bioreactor” system for delivery of transgenic proteins to a primate brain. It also appears that the physical prevention of host-cell and implanted-cell contact by two layers of device membrane provide a means whereby implanted cells can be sustained in the absence of immunosuppressive regimens. The result also implies that the bacterial CD gene, expressed by the implanted cells, did not result in any excessive or influencing immune response. The data indicate that both the CMV- and SV40-promoters remain active in the primate brain, because the expression of immunoreactive BDNF from explanted devices appeared to match that from cultured controls (CMV-promoter), and the G418 resistance was not lost after implantation in the primate brain (SV40-promoter).

One published study has indicated that xenogeneic cells in the context of an immunoisolatory device (different in design and use of materials from the device use in the present invention) did not survive in the primate brain, (Blanchet et al., , Progress in Neuro-Psychopharmacol & Biol Psychiatry 27:607, 2003). In contrast, the cells selected and used herein were found to quantitatively retain function in the current devices implanted in the primate brain.

There are numerous references in the literature that provide further background to the present invention. Some of these references are provided below and are incorporated herein by reference in their entirety. The disclosure of all references and Patents cited herein are specifically incorporated by reference in their entirety.

Aebischer, P., N. A. Pochon, et al. (1996). “Gene therapy for amyotrophic lateral sclerosis (ALS) using a polymer encapsulated xenogenic cell line engineered to secrete hCNTF.” Hum Gene Ther 7(7): 851-60.

Aebischer, P., M. Schluep, et al. (1996). “Intrathecal delivery of CNTF using encapsulated genetically modified xenogeneic cells in amyotrophic lateral sclerosis patients.”Nat Med 2(6): 696-9.

Aebischer, P., P. A. Tresco, et al. (1991). “Long-term cross-species brain transplantation of a polymer-encapsulated dopamine-secreting cell line.” Exp Neurol 111(3): 269-75.

Arcone, R., G. Gualandi, et al. (1988). “Identification of sequences responsible for acute-phase induction of human C-reactive protein.” Nucleic Acids Res 16(8): 3195-207.

Austin, E. A. and B. E. Huber (1993). “A first step in the development of gene therapy for colorectal carcinoma: cloning, sequencing, and expression of Escherichia coli cytosine deaminase.” Mol Pharmacol 43(3): 380-7.

Baroni, M. G., M. G. Cavallo, et al. (1999). “Beta-cell gene expression and functional characterisation of the human insulinoma cell line CM.” J Endocrinol 161(1): 59-68.

Baskar, J. F., P. P. Smith, et al. (1996). “Developmental analysis of the cytomegalovirus enhancer in transgenic animals.” J Virol 70(5): 3215-26.

Baskar, J. F., P. P. Smith, et al. (1996). “The enhancer domain of the human cytomegalovirus major immediate-early promoter determines cell type-specific expression in transgenic mice.” J Virol 70(5): 3207-14.

Benvenisty, N. and L. Reshef (1986). “Direct introduction of genes into rats and expression of the genes.” Proc Natl Acad Sci USA 83(24): 9551-5.

Bitter, G. A., K. M. Egan, et al. (1987). “Expression and secretion vectors for yeast.” Methods Enzymol 153: 516-44.

Blanchet, P. J., S. Konitsiotis, et al. (2003). “Complications of a trophic xenotransplant approach in parkinsonian monkeys.” Prog Neuropsychopharmacol Biol Psychiatry 27(4): 607-12.

Cavallo, M. G., F. Dotta, et al. (1996). “Beta-cell markers and autoantigen expression by a human insulinoma cell line: similarities to native beta cells.” J Endocrinol 150(1): 113-20.

Chen, C. and H. Okayama (1987). “High-efficiency transformation of mammalian cells by plasmid DNA.” Mol Cell Biol 7(8): 2745-52.

Clark, S. A., C. Quaade, et al. (1997). “Novel insulinoma cell lines produced by iterative engineering of GLUT2, glucokinase, and human insulin expression.” Diabetes 46(6): 958-67.

Dachs, G. U., A. V. Patterson, et al. (1997). “Targeting gene expression to hypoxic tumor cells.” Nat Med 3(5): 515-20.

Duan, D., H. Yang, et al. (2005). “Long-term restoration of nigrostriatal system function by implanting GDNF genetically modified fibroblasts in a rat model of Parkinson's disease.” Exp Brain Res 161(3): 316-24.

Dubensky, T. W., B. A. Campbell, et al. (1984). “Direct transfection of viral and plasmid DNA into the liver or spleen of mice.” Proc Natl Acad Sci USA 81(23): 7529-33.

Emerich, D. F., M. D. Lindner, et al. (1996). “Implants of encapsulated human CNTF-producing fibroblasts prevent behavioral deficits and striatal degeneration in a rodent model of Huntington's disease.” J Neurosci 16(16): 5168-81.

Evers, B. M., C. M. Townsend, Jr., et al. (1991). “Establishment and characterization of a human carcinoid in nude mice and effect of various agents on tumor growth.” Gastroenterology 101(2): 303-11.

Fechheimer, M., J. F. Boylan, et al. (1987). “Transfection of mammalian cells with plasmid DNA by scrape loading and sonication loading.” Proc Natl Acad Sci USA 84(23): 8463-7.

Felgner, P. L. (1996). “Improvements in cationic liposomes for in vivo gene transfer.” Hum Gene Ther 7(15): 1791-3.

Felgner, P. L. (1997). “Nonviral strategies for gene therapy.” Sci Am 276(6): 102-6.

Ferkol, T., G. L. Lindberg, et al. (1993). “Regulation of the phosphoenolpyruvate carboxykinase/human factor IX gene introduced into the livers of adult rats by receptor-mediated gene transfer.” Faseb J 7(11): 1081-91.

Fraley, R. T., C. S. Fornari, et al. (1979). “Entrapment of a bacterial plasmid in phospholipid vesicles: potential for gene transfer.” Proc Natl Acad Sci USA 76(7): 3348-52.

Gill, S. S., N. K. Patel, et al. (2003). “Direct brain infusion of glial cell line-derived neurotrophic factor in Parkinson disease.” Nat Med 9(5): 589-95.

Goeddel, D. V. (1990). “Systems for heterologous gene expression.” Methods Enzymol 185: 3-7.

Gopal, T. V. (1985). “Gene transfer method for transient gene expression, stable transformation, and cotransformation of suspension cell cultures.” Mol Cell Biol 5(5): 1188-90.

Gossen, M. and H. Bujard (1992). “Tight control of gene expression in mammalian cells by tetracycline-responsive promoters.” Proc Natl Acad Sci USA 89(12): 5547-51.

Gossen, M., S. Freundlieb, et al. (1995). “Transcriptional activation by tetracyclines in mammalian cells.” Science 268(5218): 1766-9.

Graham, F. L. and A. J. van der Eb (1973). “A new technique for the assay of infectivity of human adenovirus 5 DNA.” Virology 52(2): 456-67.

Grem, J. L., K. D. Danenberg, et al. (2001). “Thymidine kinase, thymidylate synthase, and dihydropyrimidine dehydrogenase profiles of cell lines of the National Cancer Institute's Anticancer Drug Screen.” Clin Cancer Res 7(4): 999-1009.

Harland, R. and H. Weintraub (1985). “Translation of mRNA injected into Xenopus oocytes is specifically inhibited by antisense RNA.” J Cell Biol 101(3): 1094-9.

Henry, J. B. (2001). Clinical Diagnosis and Management by Laboratory Methods, W. B. Saunders Company.

Hohmeier, H. E., H. BeltrandelRio, et al. (1997). “Regulation of insulin secretion from novel engineered insulinoma cell lines.” Diabetes 46(6): 968-77.

Huber, B. E., E. A. Austin, et al. (1993). “In vivo antitumor activity of 5-fluorocytosine on human colorectal carcinoma cells genetically modified to express cytosine deaminase.” Cancer Res 53(19): 4619-26.

Innominato, P. F., L. Libbrecht, et al. (2001). “Expression of neurotrophins and their receptors in pigment cell lesions of the skin.” J Pathol 194(1): 95-100.

Johnston, R. E., O. Dillon-Carter, et al. (2001). “Trophic factor secreting kidney cell lines: in vitro characterization and functional effects following transplantation in ischemic rats.” Brain Res 900(2): 268-76.

Kageyama, R., N. Kitamura, et al. (1987). “Differing utilization of homologous transcription initiation sites of rat K and T kininogen genes under inflammation condition.” J Biol Chem 262(5): 2345-51.

Kaneda, Y., K. Iwai, et al. (1989). “Increased expression of DNA cointroduced with nuclear protein in adult rat liver.” Science 243(4889): 375-8.

Kanuga, N., H. L. Winton, et al. (2002). “Characterization of genetically modified human retinal pigment epithelial cells developed for in vitro and transplantation studies.” Invest Ophthalmol Vis Sci 43(2): 546-55.

Kato, K., M. Nakanishi, et al. (1991). “Expression of hepatitis B virus surface antigen in adult rat liver. Co-introduction of DNA and nuclear protein by a simplified liposome method.” J Biol Chem 266(6): 3361-4.

Kirschmeier, P. T., G. M. Housey, et al. (1988). “Construction and characterization of a retroviral vector demonstrating efficient expression of cloned cDNA sequences.” DNA 7(3): 219-25.

Lane, S. B., K. T., Tchedre et al., (2004). “characterization of lecithin:cholesterol acyltransferase expressed in a human lung cell line.” Protein Expr. Purif., 36(2): 157-164.

Levin, B. E., V. H. Routh, et al. (2004). “Neuronal Glucosensing.” Diabetes 53: 2521-2528.

Lindner, M. D., S. R. Winn, et al. (1995). “Implantation of encapsulated catecholamine and GDNF-producing cells in rats with unilateral dopamine depletions and parkinsonian symptoms.” Exp Neurol 132(1): 62-76.

Macejak, D. G. and P. Sarnow (1991). “Internal initiation of translation mediated by the 5′ leader of a cellular mRNA.” Nature 353(6339): 90-4.

Miller, C. R., C. R. Williams, et al. (2002). “Intratumoral 5-fluorouracil produced by cytosine deaminase/5-fluorocytosine gene therapy is effective for experimental human glioblastomas.” Cancer Res 62(3): 773-80.

Monetini, L., M. G. Cavallo, et al. (1999). “T cell reactivity to human insulinoma cell line (CM) antigens in patients with type 1 diabetes.” Autoimmunity 29(3): 171-7.

Mowla, S. J., H. F. Farhadi, et al. (2001). “Biosynthesis and post-translational processing of the precursor to brain-derived neurotrophic factor.” J Biol Chem 276(16): 12660-6.

Mullen, C. A., M. Kilstrup, et al. (1992). “Transfer of the bacterial gene for cytosine deaminase to mammalian cells confers lethal sensitivity to 5-fluorocytosine: a negative selection system.” Proc Natl Acad Sci USA 89(1): 33-7.

Nicolau, C. and C. Sene (1982). “Liposome-mediated DNA transfer in eukaryotic cells. Dependence of the transfer efficiency upon the type of liposomes used and the host cell cycle stage.” Biochim Biophys Acta 721(2): 185-90.

Oliviero, S., G. Morrone, et al. (1987). “The human haptoglobin gene: transcriptional regulation during development and acute phase induction.” Embo J 6(7): 1905-12.

Onn, A., T. Isobe, et al. (2003). “Development of an orthotopic model to study the biology and therapy of primary human lung cancer in nude mice.” Clin Cancer Res 9(15): 5532-9.

Pelletier, J. and N. Sonenberg (1988). “Internal initiation of translation of eukaryotic mRNA directed by a sequence derived from poliovirus RNA.” Nature 334(6180): 320-5.

Perales, J. C., T. Ferkol, et al. (1994). “Gene transfer in vivo: sustained expression and regulation of genes introduced into the liver by receptor-targeted uptake.” Proc Natl Acad Sci USA 91(9): 4086-90.

Poli, V. and R. Cortese (1989). “Interleukin 6 induces a liver-specific nuclear protein that binds to the promoter of acute-phase genes.” Proc Natl Acad Sci USA 86(21): 8202-6.

Potter, H., L. Weir, et al. (1984). “Enhancer-dependent expression of human kappa immunoglobulin genes introduced into mouse pre-B lymphocytes by electroporation.” Proc Natl Acad Sci USA 81(22): 7161-5.

Price, J., D. Turner, et al. (1987). “Lineage analysis in the vertebrate nervous system by retrovirus-mediated gene transfer.” Proc Natl Acad Sci USA 84(1): 156-60.

Prowse, K. R. and H. Baumann (1988). “Hepatocyte-stimulating factor, beta 2 interferon, and interleukin-1 enhance expression of the rat alpha 1-acid glycoprotein gene via a distal upstream regulatory region.” Mol Cell Biol 8(1): 42-51.

Radler, J. O., I. Koltover, et al. (1997). “Structure of DNA-cationic liposome complexes: DNA intercalation in multilamellar membranes in distinct interhelical packing regimes.” Science 275(5301): 810-4.

Ricci, A., S. Greco, et al. (2000). “Neurotrophins and neurotrophin receptors in human pulmonary arteries.” J Vasc Res 37(5): 355-63.

Rippe, R. A., D. A. Brenner, et al. (1990). “DNA-mediated gene transfer into adult rat hepatocytes in primary culture.” Mol Cell Biol 10(2): 689-95.

Ron, D., A. R. Brasier, et al. (1991). “Angiotensinogen gene-inducible enhancer-binding protein 1, a member of a new family of large nuclear proteins that recognize nuclear factor kappa B-binding sites through a zinc finger motif.” Mol Cell Biol 11(5): 2887-95.

Sajadi, A., J. C. Bensadoun, et al. (2005). “Transient striatal delivery of GDNF via encapsulated cells leads to sustained behavioral improvement in a bilateral model of Parkinson disease.” Neurobiol Dis.

Scharf, K. D., T. Materna, et al. (1994). “Heat stress promoters and transcription factors.” Results Probl Cell Differ 20: 125-62.

Shangguan, T., D. Cabral-Lilly, et al. (2000). “A novel N-acyl phosphatidylethanolamine-containing delivery vehicle for spermine-condensed plasmid DNA.” Gene Ther 7(9): 769-83.

Smith, G. P. and C. R. Kjeldsberg (2001). Cerebrospinal, Synovial, and Serous Body Fluids. Clinical Diagnosis and Management by Laboratory Methods. J. B. Henry. Philadelphia, W. B. Saunders Company: 403-424.

Smith, R. L., D. L. Traul, et al. (2000). “Characterization of promoter function and cell-type-specific expression from viral vectors in the nervous system.” J Virol 74(23): 11254-61.

Thanos, C. G., W. J. Bell, et al. (2004). “Sustained secretion of ciliary neurotrophic factor to the vitreous, using the encapsulated cell therapy-based NT-501 intraocular device. ” Tissue Eng 10(11-12): 1617-22.

Tiraby, M., C. Cazaux, et al. (1998). “Concomitant expression of E. coli cytosine deaminase and uracil phosphoribosyltransferase improves the cytotoxicity of 5-fluorocytosine.” FEMS Microbiol Lett 167(1): 41-9.

Townsend, C. M., Jr., J. Ishizuka, et al. (1993). “Studies of growth regulation in a neuroendocrine cell line.” Acta Oncol 32(2): 125-30.

Tresco, P. A., S. R. Winn, et al. (1992). “Polymer encapsulated neurotransmitter secreting cells. Potential treatment for Parkinson's disease.” Asaio J 38(1): 17-23.

Tur-Kaspa, R., L. Teicher, et al. (1986). “Use of electroporation to introduce biologically active foreign genes into primary rat hepatocytes.” Mol Cell Biol 6(2): 716-8.

Wagner, E., M. Zenke, et al. (1990). “Transferrin-polycation conjugates as carriers for DNA uptake into cells.” Proc Natl Acad Sci USA 87(9): 3410-4.

Wilson, D. R., T. S. Juan, et al. (1990). “A 58-base-pair region of the human C3 gene confers synergistic inducibility by interleukin-1 and interleukin-6. ” Mol Cell Biol 10(12): 6181-91.

Winn, S. R., P. A. Tresco, et al. (1991). “Behavioral recovery following intrastriatal implantation of microencapsulated PC12 cells.” Exp Neurol 113(3): 322-9.

Wolters, G. H., W. M. Fritschy, et al. (1991). “A versatile alginate droplet generator applicable for microencapsulation of pancreatic islets.” J Appl Biomater 3(4): 281-6.

Wu, G. Y. and C. H. Wu (1987). “Receptor-mediated in vitro gene transformation by a soluble DNA carrier system.” J Biol Chem 262(10): 4429-32.

Wu, G. Y. and C. H. Wu (1988). “Evidence for targeted gene delivery to Hep G2 hepatoma cells in vitro.” Biochemistry 27(3): 887-92.

Yang, N. S., J. Burkholder, et al. (1990). “In vivo and in vitro gene transfer to mammalian somatic cells by particle bombardment.” Proc Natl Acad Sci USA 87(24): 9568-72.

Zechner, R., T. C. Newman, et al. (1988). “Recombinant human cachectin/tumor necrosis factor but not interleukin-1 alpha downregulates lipoprotein lipase gene expression at the transcriptional level in mouse 3T3-L1 adipocytes.” Mol Cell Biol 8(6): 2394-401.

Zermansky, A. J., F. Bolognani, et al. (2001). “Towards global and long-term neurological gene therapy: unexpected transgene dependent, high-level, and widespread distribution of HSV-1 thymidine kinase throughout the CNS.” Mol Ther 4(5): 490-8.

What is claimed is:

1. A CRL 5803 cell genetically modified by transformation with one or more expression constructs, each construct comprising a nucleic acid encoding a transgene operatively linked to a promoter, wherein said cell expresses at least a first transgene, a second transgene, a third transgene and a fourth transgene,
wherein at least one of said first, second, third or fourth transgenes encodes a therapeutic polypeptide and
wherein said cell expresses said therapeutic polypeptide for at least 40 population doublings.

2. The cell of claim 1, further comprising at least one selectable marker.

3. The cell of claim 1, wherein said therapeutic polypeptide is selected from the group consisting of a of a therapeutic polypeptide growth factor, an enzyme, a cytokine, a tumor suppressor, an apoptosis inducer, a hormone, a hematopoietic factor, a hemostasis factor, a pressor molecule, a receptor, a transporter protein, and a channel protein.

4. The cell of claim 2, wherein said first transgene is a neurotrophic factor gene, said second transgene is secreted alkaline phosphatase (SEAP) gene, said third transgene is a wild type cytosine deaminase gene, and said fourth transgene is a neurotrophic factor gene, said marker gene is a neomycin phosphotransferase gene, and wherein said cell is further transformed with a hygromycin phosphotransferase gene.

5. The cell of claim 1, wherein said promoter is heterologous to said nucleic acid encoding said therapeutic polypeptide.

6. The cell of claim 1, wherein said promoter is homologous to said nucleic acid encoding said therapeutic polypeptide.

7. The cell of claim 1, wherein said promoter is selected from the group consisting of a constitutive promoter, a tissue-specific promoter, an inducible promoter, and a non-inducible promoter.

8. The cell of claim 1, wherein said promoter is selected from the group consisting of a human cytomegalovirus (CMV) immediate early gene promoter, a SV40 early promoter, a Rous sarcoma virus long terminal repeat, a rat insulin promoter, an EF-1 α promoter, an Ubiquitin promoter, and a glyceraldehyde-3-phosphate dehydrogenase promoter.

9. A CRL 5803 cell deposited with DSMZ under accession number DSM ACC2730.

10. A composition comprising the cell of claim 1 and a pharmaceutically acceptable carrier, excipient or diluent.

11. A composition comprising the cell of claim 9 and a pharmaceutically acceptable carrier, excipient or diluent.

12. A method of treatment comprising the step of implanting in a subject the cell according to claim 1, wherein said cell expresses said therapeutic polypeptide in amount effective to treat a condition treatable with said therapeutic polypeptide.

13. The method of claim 12, wherein said cell is implanted in said subject's central nervous system.

14. The method of claim 13, wherein said cell is implanted in said subject's brain.

15. The method of claim 13, wherein said cell is implanted in said subject's spinal cord.

16. The method of claim 13, wherein said cell is implanted in a tumor of said subject.

17. The method of claim 12, further comprising the step of administering an immunosuppressant.

18. The method of claim 12, further comprising the step of administering an anti-viral agent.

19. The method of claim 12, further comprising the step of administering an anti-bacterial agent.

20. The method of claim 12, wherein said subject is a human.

21. The method of claim 12, wherein said therapeutic polypeptide is selected from the group consisting of a growth factor, an enzyme, a cytokine, a tumor suppressor, an apoptosis inducer, a hormone, a hematopoietic factor, hemostasis factor, and a pressor molecule.

22. The method of claim 12, wherein said cell is implanted in said subject in a cell density of about 1×10⁷ cells/ml to about 1×10⁹ cells/ml.

23. A method of treating a central nervous system (CNS) disorder in a subject comprising implanting the cell according to claim 1 in said subject, wherein said cell expresses a therapeutic polypeptide in amount effective to treat said CNS disorder.

24. The method of claim 23, wherein said cell is implanted said subject's brain.

25. The method of claim 23, wherein said cell is implanted in said subject's spinal cord.

26. The method of claim 23, further comprising the step of administering an immunosuppressant.

27. The method of claim 23, further comprising the step of administering an anti-viral agent.

28. The method of claim 23, further comprising the step of administering an anti-bacterial agent.

29. The method of claim 23, wherein said subject is a human.

30. The method of claim 23, wherein said therapeutic polypeptide is selected from the group consisting of a growth factor, an enzyme, an antibody, a cytokine, a tumor suppressor, an apoptosis inducer, a hormone, a hematopoietic factor, hemostasis factor, and a pressor molecule.

31. The method of claim 23, wherein said cell is implanted in said subject in a cell density of about 1×10⁷ cells/ml to about 1×10⁹ cells/ml.

32. A method of delivering a therapeutic polypeptide to a site in a human body comprising implanting at said site the cell according to claim 1, wherein said cell expresses said therapeutic polypeptide.

33. The method of claim 32, wherein said site is a central nervous system site.

34. The method of claim 33, wherein said site is a brain.

35. The method of claim 33, wherein said site is a spinal cord site.

36. The method of claim 32, wherein said site is a tumor.

37. The method of claim 32, wherein said therapeutic agent is selected from the group consisting of a growth factor, an enzyme, a cytokine, a tumor suppressor, an apoptosis inducer, a hormone, a hematopoietic factor, hemostasis factor, and a pressor molecule.

38. The method of claim 32, wherein said cell is implanted in said body in a cell density of about 1×10⁷ cells/ml to about 1×10⁹ cells/ml.

39. A method for sustaining in vitro production of a therapeutic polypeptide comprising culturing the cell of claim 1 under conditions that allow production of said polypeptide for at least 40 population doublings.

40. A human cell engineered with a cytosine deaminase expression construct derived from a wild-type cytosine deaminase bacterial gene sequence operably linked to a promoter element, wherein the cytosine deaminase expression construct expresses a protein comprising the amino acid sequence set forth in SEQ ID NO: 11.

41. The cell of claim 40, wherein said cell is a CRL5803 cell.

42. A composition comprising the cell of claim 40 and a pharmaceutically acceptable carrier, excipient or diluent.

43. An implantable system comprising the cell of claim 1, wherein said system is immobilized at an implantation site to maintain said cell at said implantation site and permit diffusion of an expressed and secreted therapeutic polypeptide from said implantation site.

44. The system of claim 43, wherein said therapeutic polypeptide is selected from the group of a therapeutic polypeptide growth factor, an enzyme, a cytokine, a tumor suppressor, an apoptosis inducer, a hormone, a hematopoietic factor, hemostasis factor, a pressor molecule.

45. The system of claim 43, wherein said site is a subject's central nervous system

46. The system of claim 43, wherein said site is a subject's brain.

47. The system of claim 43, wherein said site is a subject's spinal cord.

48. The system of claim 43, wherein said site is a tumor.

49. The system of claim 43, wherein said system comprises a cell density between about 1×10⁷ cells/ml to about 1×10⁹ cells/ml device volume.

50. The system of claim 43, wherein said cell survives under culture conditions or in vivo in said system for at least a month with a functionality that represents at least 80% of the function expressed at the time the cells are/were introduced into said system.

51. The system of claim 43, wherein said cell in said system expand in said system to increase in cell density and/or cell function upon implantation of the system in vivo.

52. The system of claim 43, wherein said cell in said system have been genetically engineered by transformation with an expression construct that comprises a nucleic acid that encodes a therapeutic polypeptide operatively linked to a promoter, wherein said transformed cell expresses and secretes said therapeutic polypeptide.

53. The system of claim 43, wherein said promoter is heterologous to said nucleic acid encoding said therapeutic polypeptide.

54. The system of claim 43, wherein said promoter is homologous to said nucleic acid encoding said therapeutic polypeptide.

55. The system of claim 43, wherein said promoter is selected from the group consisting of a constitutive promoter, a tissue-specific promoter, an inducible promoter, and a non-inducible promoter.

56. The system of claim 43, wherein said promoter is selected from the group consisting of a human cytomegalovirus (CMV) immediate early gene promoter, a SV40 early promoter, a Rous sarcoma virus long terminal repeat, a rat insulin promoter, an EF-1 α promoter, an Ubiquitin promoter, and a glyceraldehyde-3-phosphate dehydrogenase promoter.


SEQUENCE LISTING (12 sequences)

SEQ ID NO: 1 - DNA, 24 bp, Artificial sequence
  Synthetic oligonucleotide
cgacagcagcaatgacgcatgtgg

SEQ ID NO: 2 - DNA, 25 bp, Artificial sequence
  Synthetic oligonucleotide
cgcatccggcataaactaagctcgc

SEQ ID NO: 3 - DNA, 1296 bp, E. coli
atgtggaggctaacagtgtcgaataacgctttacaaacaattattaacgcccggttaccaggcgaagaggggctgtggcagattcatctgcaggacggaaaaatcagcgccattgatgcgcaatccggcgtgatgcccataactgaaaacagcctggatgccgaacaaggtttagttataccgccgtttgtggagccacatattcacctggacaccacgcaaaccgccggacaaccgaactggaatcagtccggcacgctgtttgaaggcattgaacgctgggccgagcgcaaagcgttattaacccatgacgatgtgaaacaacgcgcatggcaaacgctgaaatggcagattgccaacggcattcagcatgtgcgtacccatgtcgatgtttcggatgcaacgctaactgcgctgaaagcaatgctggaagtgaagcaggaagtcgcgccgtggatcgatctgcaaatcgtcgccttccctcaggaagggattttgtcgtatcccaacggtaaagcgttgctggaagaggcgttacgcttaggggcagatgtagtgggggcgattccgcattttgaatttacccgtgaatacggcgtggagtcgctgcatagaaccttcgccctggcgcaaaaatacgaccgtctcatcgacgttcactgtgatgagatcgatgacgagcaatcgcgctttgtcgaaaccgttgctgccctggcgcaccatgaaggcatgggcgcgcgagtcaccgccagccacaccacggcaatgcactcctataacggggcgtatacctcacgcctgttccgcttgctgaaaatgtccggtattaactttgtcgccaacccgctggtcaatattcatctgcaaggacgtttcgatacgtatccaaaacgtcgcggcaccacgcgcgttaaagagatgctggagtccggcattaacgtctgctttggtcacgatgatgtcttcgatccgtggtatccgctgggaacggcgaatatgctgcaagtgctgcatatggggctgcatgtttgccagttgatgggctacgggcagattaacgatggcctgaatttaatcacccaccacagcgcaaggacgttgaatttgcaggattacggcattgccgccggaaacagcgccaacctgattatcctgccggctgaaaatgggtttgatgcgctgcgccgtcaggttccggtacgttattcggtacgtggcggcaaggtgattgccagcacacaaccggcacaaaccaccgtatatttggagcagccagaagccatcgattacaaacgt

SEQ ID NO: 4 - DNA, 36 bp, Artificial sequence
  Synthetic oligonucleotide
atgcgtcgacagcagcaaggacgcatgtggagccta

SEQ ID NO: 5 - DNA, 24 bp, Artificial sequence
  Synthetic oligonucleotide
cgccgttcccagcggataccacgg

SEQ ID NO: 6 - DNA, 24 bp, Artificial sequence
  Synthetic oligonucleotide
aatgacgtcgacatgtggaggcta

SEQ ID NO: 7 - DNA, 7283 bp, Artificial sequence
  Synthetic oligonucleotide
cccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggcgaccgcccagcgacccccgcccgttgacgtcaatagtgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctagcattatgcccagtacatgaccttacgggagtttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatatagcagagctcgtttagtgaaccgtcagaattaattcagatcagcttgcatgcctgcaggtcgacatgtggaggctaacagtgtcgaataacgctttacaaacaattattaacgcccggttaccaggcgaagaggggctgtggcagattcatctgcaggacggaaaaatcagcgccattgatgcgcaatccggcgtgatgcccataactgaaaacagcctggatgccgaacaaggtttagttataccgccgtttgtggagccacatattcacctggacaccacgcaaaccgccggacaaccgaactggaatcagtccggcacgctgtttgaaggcattgaacgctgggccgagcgcaaagcgttattaacccatgacgatgtgaaacaacgcgcatggcaaacgctgaaatggcagattgccaacggcattcagcatgtgcgtacccatgtcgatgtttcggatgcaacgctaactgcgctgaaagcaatgctggaagtgaagcaggaagtcgcgccgtggatcgatctgcaaatcgtcgccttccctcaggaagggattttgtcgtatcccaacggtaaagcgttgctggaagaggcgttacgcttaggggcagatgtagtgggggcgattccgcattttgaatttacccgtgaatacggcgtggagtcgctgcatagaaccttcgccctggcgcaaaaatacgaccgtctcatcgacgttcactgtgatgagatcgatgacgagcaatcgcgctttgtcgaaaccgttgctgccctggcgcaccatgaaggcatgggcgcgcgagtcaccgccagccacaccacggcaatgcactcctataacggggcgtatacctcacgcctgttccgcttgctgaaaatgtccggtattaactttgtcgccaacccgctggtcaatattcatctgcaaggacgtttcgatacgtatccaaaacgtcgcggcaccacgcgcgttaaagagatgctggagtccggcattaacgtctgctttggtcacgatgatgtcttcgatccgtggtatccgctgggaacggcgaatatgctgcaagtgctgcatatggggctgcatgtttgccagttgatgggctacgggcagattaacgatggcctgaatttaatcacccaccacagcgcaaggacgttgaatttgcaggattacggcattgccgccggaaacagcgccaacctgattatcctgccggctgaaaatgggtttgatgcgctgcgccgtcaggttccggtacgttattcggtacgtggcggcaaggtgattgccagcacacaaccggcacaaaccaccgtatatttggagcagccagaagccatcgattacaaacgttgaacgactgggttacagcgagcttagtttatgccggatgcggacggatcctctagaggatccccgggtggcatccctgtgacccctccccagtgcctctcctggccttggaagttgccactccagtgcccaccagccttgtcctaataaaattaagttgcatcattttgtctgactaggtgtcctctataatattatggggtggaggggggtggtatggagcaaggggcccaagttgggaagacaacctgtagggcctgcggggtctattcgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctgccatggcccaaccggtgggacatttgagttgcttgcttggcactgtcctctcatgcgttgggtccactcagtagatgcctgttgaattgggtacgcggccagcttctgtggaatgtgtgtcagttagggtgtggaaagtccccaggctccccagcaggcagaagtatgcaaagcatgcatctcaattagtcagcaaccaggtgtggaaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggaattgatccagcagacgctcgtccgggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgaacaagagctgatttcgagaacgcctcagcggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaattccctatagtgagtcgtattaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacaaaatattaacgtttacaattt

SEQ ID NO: 8 - protein, 30 aa, Artificial sequence
  Synthetic peptide
PPFVEPHIHLDTTQTAGQPNWNQSGTLFEG

SEQ ID NO: 9 - protein, 25 aa, Artificial sequence
  Synthetic peptide
LVNIHLQGRFDTYPKRRGITRVKEM

SEQ ID NO: 10 - DNA, 1296 bp, Artificial sequence; identical to SEQ ID NO: 3
  Synthetic polynucleotide

SEQ ID NO: 11 - protein, 432 aa, Artificial sequence
  Synthetic peptide
MWRLTVSNNALQTIINARLPGEEGLWQIHLQDGKISAIDAQSGVMPITENSLDAEQGLVIPPFVEPHIHLDTTQTAGQPNWNQSGTLFEGIERWAERKALLTHDDVKQRAWQTLKWQIANGIQHVRTHVDVSDATLTALKAMLEVKQEVAPWIDLQIVAFPQEGILSYPNGKALLEEALRLGADVVGAIPHFEFTREYGVESLHRTFALAQKYDRLIDVHCDEIDDEQSRFVETVAALAHHEGMGARVTASHTTAMHSYNGAYTSRLFRLLKMSGINFVANPLVNIHLQGRFDTYPKRRGTTRVKEMLESGINVCFGHDDVFDPWYPLGTANMLQVLHMGLHVCQLMGYGQINDGLNLITHHSARTLNLQDYGIAAGNSANLIILPAENGFDALRRQVPVRYSVRGGKVIASTQPAQTTVYLEQPEAIDYKR

SEQ ID NO: 12 - protein, 427 aa, E. coli
MSNNALQTIINARLPGEEGLWQIHLQDGKISAIDAQSGVMPITENSLDAEQGLVIPPFVEPHIHLDTTQTAGQPNWNQSGTLFEGIERWAERKALLTHDDVKQRAWQTLKWQIANGIQHVRTHVDVSDATLTALKAMLEVKQEVAPWIDLQIVAFPQEGILSYPNGKALLEEALRLGADVVGAIPHFEFTREYGVESLHRTFALAQKYDRLIDVHCDEIDDEQSRFVETVAALAHHEGMGARVTASHTTAMHSYNGAYTSRLFRLLKMSGINFVANPLVNIHLQGRFDTYPKRRGTTRVKEMLESGINVCFGHDDVFDPWYPLGTANMLQVLHMGLHVCQLMGYGQINDGLNLITHHSARTLNLQDYGIAAGNSANLIILPAENGFDALRRQVPVRYSVRGGKVIASTQPAQTTVYLEQPEAIDYKR